(19) United States
(12) Patent Application Publication
(10) Pub. No.: US 2026/0308209 A1
(43) Pub. Date: Oct. 8, 2026

(54) OPTIMIZED CSP VARIANTS AND RELATED METHODS

(71) Applicant: BioNTech SE, Mainz (DE)

(72) Inventors: Thomas ZIEGENHALS, Mainz (DE); Charles JENNISON, Mainz (DE); Karla-Gerlinde SCHRAUT, Mainz (DE); Anja DOKIC, Mainz (DE); Svenja GROBE, Mainz (DE); Patricia DOS SANTOS MEIRELES, Mainz (DE); Ugur SAHIN, Mainz (DE)

(21) Appl. No.: 19/480,587

(22) PCT Filed: May 2, 2024
(86) PCT No.: PCT/IB2024/054273

§ 371 Date: Oct. 31, 2025

Related U.S. Application Data
(60) Provisional application No. 63/515,321, filed on Jul. 24, 2023.
(60) Provisional application No. 63/586,224, filed on Sep. 28, 2023.

(30) Foreign Application Priority Data
May 3, 2023 (WO) PCT/IB2023/054622

(51) Int. Cl. C07K 14/445; A61K 39/00; A61P 31/00; A61P 37/04; C12N 15/88

(52) U.S. Cl. CPC C07K 14/445; A61P 31/00; A61P 37/04; C12N 15/88; A61K 39/00; A61K 2039/53; A61K 2039/55555

(57) ABSTRACT

The present disclosure provides compositions (e.g., pharmaceutical compositions) for delivery of Plasmodium circumsporozoite protein (CSP) antigens and related technologies (e.g., components thereof and/or methods relating thereto). Among other things, the present disclosure provides polyribonucleotides comprising a coding sequence that encodes a full-length Plasmodium CSP polypeptide. In some embodiments, a full-length Plasmodium CSP polypeptide comprises a Plasmodium CSP N-terminal domain, and the coding sequence of a polyribonucleotide encoding the full-length Plasmodium CSP polypeptide has a particular nucleotide content as provided herein.

### CROSS REFERENCE TO RELATED APPLICATIONS

The present application claims priority to International Application No. PCT/IB23/54622, filed May 3, 2023, U.S. Provisional Patent Application No. 63/515,321, filed Jul. 24, 2023, and U.S. Provisional Patent Application No. 63/586,224, filed Sep. 28, 2023, each of which is hereby incorporated by reference in its entirety.

### SEQUENCE LISTING

The present specification makes reference to a Sequence Listing (submitted electronically as a .xml file named “2013237-1047.xml” on May 2, 2024). The .xml file was generated on Apr. 26, 2024, and is 126,597 bytes in size. The entire contents of the Sequence Listing are herein incorporated by reference.

### BACKGROUND

Malaria is a mosquito-borne infectious disease caused by protozoan parasites of the Plasmodium genus. According to the World Health Organization, an estimated 3.4 billion people in 92 countries are at risk of being infected with the Plasmodium parasite and developing disease.

### SUMMARY OF THE INVENTION

The present disclosure provide technologies (e.g., compositions, methods, etc.) for delivery of Plasmodium antigens (also referred to herein as “malaria antigens” or “Plasmodium antigens”). In particular, the present disclosure provides technologies (e.g., compositions, methods, etc.) for delivery of a Plasmodium circumsporozoite protein (CSP) antigen. The present disclosure identifies a problem with certain polyribonucleotides that encode a full-length Plasmodium CSP polypeptide (including a Plasmodium CSP secretory signal and Plasmodium CSP GPI anchor), in that the polyribonucleotides are characterized by an irregular peak (e.g., having a shoulder or a double peak), for example, when analyzed by electrophoresis (e.g., capillary electrophoresis, e.g., using a fragment analyzer). The present disclosure encompasses a recognition that such an irregular peak is inconsistent with standards of good manufacturing practices. For example, in some embodiments, RNA integrity cannot be confirmed if there is an irregular peak. In some embodiments, an irregular peak may disguise RNA degradation and/or a contaminant. The present disclosure provides polyribonucleotides that encode a full-length Plasmodium CSP polypeptide having certain characteristics that address this problem.

The present disclosure provides the insight that the nucleotide content within a portion of a polyribonucleotide, a portion of a polyribonucleotide encoding the N-terminal region of the Plasmodium CSP polypeptide, can affect peak shape. In some embodiments, the present disclosure provides polyribonucleotides that encode a full-length Plasmodium CSP polypeptide, where nucleotide content of the portion encoding the N-terminal region of the Plasmodium CSP polypeptide is within defined ranges. In some embodiments, provided polyribonucleotides have beneficial characteristics for manufacturing. In some embodiments, the present disclosure provides polyribonucleotides that encode a full-length Plasmodium CSP polypeptide whose integrity can be confirmed using capillary electrophoresis (e.g., a fragment analyzer).

In some embodiments, the present disclosure provides polyribonucleotides comprising a coding sequence that encodes a full-length Plasmodium CSP polypeptide. In some embodiments, a full-length Plasmodium CSP polypeptide comprises a Plasmodium CSP N-terminal domain. In some embodiments, a coding sequence has an adenine content that is between 35% and 42%, and a portion of the coding sequence that encodes the Plasmodium CSP N-terminal domain has an adenine content that is at least 35%. In some embodiments, the portion of the coding sequence that encodes the Plasmodium CSP N-terminal domain has an adenine content that is between 35% and 45%. In some embodiments, the portion of the coding sequence that encodes the Plasmodium CSP N-terminal domain has an adenine content that is between 36% and 42%. In some embodiments, the coding sequence has an adenine content that is between that is between 35% and 36.5%.

In some embodiments, the coding sequence has a uracil content that is between 14% and 20%, and the portion of the coding sequence that encodes the Plasmodium CSP N-terminal domain has a uracil content that is at least 10%. In some embodiments, the coding sequence has a guanine content that is between 15% and 19.5%, and the portion of the coding sequence encoding a Plasmodium CSP N-terminal domain has a guanine content that is less than 27%. In some embodiments, the coding sequence has a cytosine content that is between 22% and 31%, and the portion of the coding sequence that encodes the Plasmodium CSP N-terminal domain has a cytosine content that is less than 28%.

In some embodiments, the present disclosure provides polyribonucleotides comprising a coding sequence that encodes a full-length Plasmodium CSP polypeptide. In some embodiments, a full-length Plasmodium CSP polypeptide comprises a Plasmodium CSP N-terminal domain. In some embodiments, a coding sequence has a uracil content that is between 14% and 20%, and a portion of the coding sequence that encodes the Plasmodium CSP N-terminal domain has a uracil content that is at least 10%. In some embodiments, the portion of the coding sequence that encodes the Plasmodium CSP N-terminal domain has a uracil content that is between 12% and 25%. In some embodiments, the portion of the coding sequence that encodes the Plasmodium CSP N-terminal domain has a uracil content that is between 17% and 22%. In some embodiments, the coding sequence has a uracil content that is between that is between 15% and 16.5%.

In some embodiments, the coding sequence has an adenine content that is between 35% and 42%, and the portion of the coding sequence that encodes the Plasmodium CSP N-terminal domain has an adenine content that is at least 35%. In some embodiments, the coding sequence has a guanine content that is between 15% and 19.5%, and the portion of the coding sequence encoding a Plasmodium CSP N-terminal domain has a guanine content that is less than 27%. In some embodiments, the coding sequence has a cytosine content that is between 22% and 31%, and the portion of the coding sequence that encodes the Plasmodium CSP N-terminal domain has a cytosine content that is less than 28%.

In some embodiments, the present disclosure provides polyribonucleotides comprising a coding sequence that encodes a full-length Plasmodium CSP polypeptide. In some embodiments, a full-length Plasmodium CSP polypeptide comprises a Plasmodium CSP N-terminal domain. In some embodiments, a coding sequence has a guanine content that is between 15% and 19.5%, and a portion of the coding sequence encoding a Plasmodium CSP N-terminal domain has a guanine content that is less than 27%. In some embodiments, the portion of the coding sequence that encodes the Plasmodium CSP N-terminal domain has a guanine content that is between 19% and 26%. In some embodiments, the portion of the coding sequence that encodes the Plasmodium CSP N-terminal domain has a guanine content that is between 19.5% and 24.5%. In some embodiments, the coding sequence has a guanine content that is between that is between 18.5% and 19.5%.

In some embodiments, the coding sequence has an adenine content that is between 35% and 42%, and the portion of the coding sequence that encodes the Plasmodium CSP N-terminal domain has an adenine content that is at least 35%. In some embodiments, the coding sequence has a uracil content that is between 14% and 20%, and the portion of the coding sequence that encodes the Plasmodium CSP N-terminal domain has a uracil content that is at least 10%. In some embodiments, the coding sequence has a cytosine content that is between 22% and 31%, and the portion of the coding sequence that encodes the Plasmodium CSP N-terminal domain has a cytosine content that is less than 28%.

In some embodiments, the present disclosure provides polyribonucleotides comprising a coding sequence that encodes a full-length Plasmodium CSP polypeptide. In some embodiments, a full-length Plasmodium CSP polypeptide comprises a Plasmodium CSP N-terminal domain. In some embodiments, a coding sequence has a cytosine content that is between 22% and 31%, and a portion of the coding sequence that encodes the Plasmodium CSP N-terminal domain has a cytosine content that is less than 28%. In some embodiments, the portion of the coding sequence that encodes the Plasmodium CSP N-terminal domain has a cytosine content that is between 12% and 27% cytosine. In some embodiments, the portion of the coding sequence that encodes the Plasmodium CSP N-terminal domain has a cytosine content that is between 15% and 22% cytosine. In some embodiments, the coding sequence has a cytosine content that is between that is between 28.5% and 30% cytosine.

In some embodiments, the coding sequence has an adenine content that is between 35% and 42%, and the portion of the coding sequence that encodes the Plasmodium CSP N-terminal domain has an adenine content that is at least 35%. In some embodiments, the coding sequence has a uracil content that is between 14% and 20%, and the portion of the coding sequence that encodes the Plasmodium CSP N-terminal domain has a uracil content that is at least 10%. In some embodiments, the coding sequence has a guanine content that is between 15% and 19.5%, and the portion of the coding sequence encoding a Plasmodium CSP N-terminal domain has a guanine content that is less than 27%.

In some embodiments, a full-length Plasmodium CSP polypeptide comprises a secretory signal, a Plasmodium CSP N-terminal domain, a Plasmodium CSP central domain, and a Plasmodium CSP C-terminal domain.

In some embodiments, a full-length Plasmodium CSP polypeptide comprises a secretory signal. In some embodiments, a full-length Plasmodium CSP polypeptide comprises a Plasmodium CSP N-terminal domain. In some embodiments, a Plasmodium CSP N-terminal domain comprises an N-terminal region, an N-terminal end region, and a junction region. In some embodiments, a full-length Plasmodium CSP polypeptide comprises a Plasmodium CSP central domain. In some embodiments, a Plasmodium CSP central domain comprises a minor repeat region and a major repeat region. In some embodiments, a full-length Plasmodium CSP polypeptide comprises a Plasmodium CSP C-terminal domain. In some embodiments, a Plasmodium CSP C-terminal domain comprises a C-terminal region and a transmembrane region.

In some embodiments, a full-length Plasmodium CSP polypeptide comprises a secretory signal. In some embodiments, a secretory signal comprises or consists of a Plasmodium secretory signal, preferably a Plasmodium CSP secretory signal.

In some embodiments, a full-length Plasmodium CSP polypeptide comprises an amino acid sequence having at least 95%, at least 96%, at least 97%, at least 98%, at least 99%, or 100% identity with the amino acid of SEQ ID NO: 1.

In some embodiments, Plasmodium is Plasmodium falciparum, preferably Plasmodium falciparum isolate 3D7.

In some embodiments, a portion of the coding sequence encoding the Plasmodium CSP N-terminal domain comprises or consists of a sequence according to any one of SEQ ID NOs: 11, 13, 15, and 17. In some embodiments, a portion of the coding sequence encoding the Plasmodium CSP N-terminal domain comprises or consists of a sequence according to SEQ ID NO: 11. In some embodiments, a portion of the coding sequence encoding the Plasmodium CSP N-terminal domain comprises or consists of a sequence according to SEQ ID NO: 13. In some embodiments, a portion of the coding sequence encoding the Plasmodium CSP N-terminal domain comprises or consists of a sequence according to SEQ ID NO: 15. In some embodiments, a portion of the coding sequence encoding the Plasmodium CSP N-terminal domain comprises or consists of a sequence according to SEQ ID NO: 17.

In some embodiments, a coding sequence comprises or consists of a sequence according to any one of SEQ ID NOs: 50, 52, 54, 56, 58, 60, and 62. In some embodiments, a coding sequence comprises or consists of a sequence according to SEQ ID NO: 50. In some embodiments, a coding sequence comprises or consists of a sequence according to SEQ ID NO: 52. In some embodiments, a coding sequence comprises or consists of a sequence according to SEQ ID NO: 54. In some embodiments, a coding sequence comprises or consists of a sequence according to SEQ ID NO: 56. In some embodiments, a coding sequence comprises or consists of a sequence according to SEQ ID NO: 58. In some embodiments, a coding sequence comprises or consists of a sequence according to SEQ ID NO: 60. In some embodiments, a coding sequence comprises or consists of a sequence according to SEQ ID NO: 62.

The present disclosure also provides RNA constructs. In some embodiments, an RNA construct comprises a polyribonucleotide provided herein.

In some embodiments, an RNA construct comprises a 5′ UTR. In some embodiments, a 5′ UTR comprises or consists of a modified human alpha-globin 5′-UTR.

In some embodiments, an RNA construct comprises a 3′ UTR. In some embodiments, a 3′ UTR comprises or consists of a first sequence from the amino terminal enhancer of split (AES) messenger RNA and a second sequence from the mitochondrial encoded 12S ribosomal RNA.

In some embodiments, an RNA construct comprises a polyA tail sequence.

In some embodiments, an RNA construct comprises in 5′ to 3′ order: (i) a 5′ UTR that comprises or consists of a modified human alpha-globin 5′-UTR; (ii) a polyribonucleotide of any one of claims 1-15; (iii) a 3′ UTR that comprises or consists of a first sequence from the amino terminal enhancer of split (AES) messenger RNA and a second sequence from the mitochondrial encoded 12S ribosomal RNA; and (iv) a polyA tail sequence.

In some embodiments, an RNA construct comprises a 5′ cap.

Provided herein are compositions. In some embodiments, a composition comprises one or more polyribonucleotides as provided herein. In some embodiments, a composition comprises one or more RNA constructs as provided herein.

In some embodiments, a composition comprises lipid nanoparticles, polyplexes (PLX), lipidated polyplexes (LPLX), or liposomes. In some embodiments, one or more polyribonucleotides are fully or partially encapsulated within lipid nanoparticles, polyplexes (PLX), lipidated polyplexes (LPLX), or liposomes.

In some embodiments, a pharmaceutical composition comprises a composition as provided herein and at least one pharmaceutically acceptable excipient.

In some embodiments, a pharmaceutical composition provided herein is for use in the treatment and/or prevention of a malaria infection that comprises administering one or more doses of the pharmaceutical composition to a subject.

The present disclosure further provides methods. In some embodiments, a method comprises administering one or more doses of the pharmaceutical composition as provided herein to a subject.

In some embodiments, the present disclosure provides use of a pharmaceutical composition as described herein in the treatment of a malaria infection, use of a pharmaceutical composition as described herein in the prevention of a malaria infection, and use of a pharmaceutical composition as described herein in inducing an anti-malaria immune response in a subject.

Combinations of polyribonucleotides are also provided herein. In some embodiments, a combination comprises a first pharmaceutical composition and a second pharmaceutical composition. In some embodiments, a first pharmaceutical composition comprises a first polyribonucleotide, where the first polyribonucleotide is a polyribonucleotide as described herein. In some embodiments, a second pharmaceutical composition comprises a second polyribonucleotide, where the second polyribonucleotide encodes a second polypeptide, and the second polypeptide comprises one or more Plasmodium T-cell antigens.

The present disclosure also provides methods comprising administering a combination provided herein to a subject.

In some embodiments, the present disclosure provides host cells comprising polyribonucleotides described herein and host cells comprising RNA constructs described herein.

Provided technologies, including exemplary polyribonucleotides, compositions comprising such polyribonucleotides, and methods of making and using such polyribonucleotides, are described in more detail herein.

### BRIEF DESCRIPTION OF THE DRAWING

The Drawing included herein, which is composed of the following Figures, is for illustration purposes only and not for limitation.

FIG. 1 depicts an electropherogram of an exemplary full-length CSP construct, RNA construct 23, that was in vitro translated at pH 7.0.

FIG. 2 depicts an electropherogram of an exemplary full-length CSP construct, RNA construct 23, that was in vitro translated at pH 8.35.

FIG. 3 depicts an electropherogram of an exemplary full-length CSP construct, RNA construct 23, that was in vitro translated at pH 7.0 and purified using various techniques: tangential flow filtration (TFF), magnetic bead purification, or oligo-dT affinity chromatography.

FIGS. 4A-4E depict electropherograms of RNA construct 23 at different time points in a forced degradation analysis: 0 minutes (FIG. 4A), 5 minutes (FIG. 4B), 10 minutes (FIG. 4C), 15 minutes (FIG. 4D), and 20 minutes (FIG. 4E).

FIG. 5 depicts an HPLC chromatogram of a sample of RNA construct 23. Three fractions of eluant were collected corresponding to the portions of the chromatogram labeled Fraction 1, Fraction 2 and Fraction 3.

FIG. 6A depicts an HPLC chromatogram of a further HPLC analysis of each of the fractions from FIG. 5. FIG. 6B depicts an HPLC chromatogram of a further HPLC analysis of pooled fractions and unfractioned sample.

FIG. 7 depicts fragment analyzer electropherograms for each of the fractions from FIG. 5.

FIG. 8 depicts fragment analyzer electropherograms for samples of the pooled fractions and unfractioned sample.

FIG. 9 depicts fragment analyzer electropherograms of RNA construct 23 and nucleotide optimized variants 6 and 7 thereof.

FIG. 10 depicts fragment analyzer electropherograms of nucleotide optimized variants 8 and 9 of RNA construct 23.

FIGS. 11A-11B depict in vitro expression of non-formulated RNA construct 23 (“23”) and different nucleotide optimized variants thereof (“V4” to “V13”) in HEK293T cells. FIG. 11A shows transfection rate as measured by percentage of HEK293T population that is positive for presence of expressed protein, providing a measure of efficiency of RNA delivery into the cell. FIG. 11B shows total expression as measured by median fluorescence of the total HEK293T population for both transfected and non-transfected cells. Permeabilized cells show total protein expressed (black bar, intracellular staining) and non-permeabilized cells show only surface expressed protein (grey bar, surface staining). NT refers to non-transfected.

FIGS. 12A-12B depict in vitro expression of RNA constructs in HEK293T cells using MessengerMax as transfection reagent. FIG. 12A shows total expression as measured by median fluorescence of the total HEK293T population for both transfected and non-transfected cells. FIG. 12B shows transfection rate as measured by percentage of total HEK293T population that is positive for presence of expressed protein. RNA construct 23 is labeled “23”; different nucleotide optimized variants are labeled “V4” to “V14”; NT refers to non-transfected; control refers to an irrelevant RNA control.

FIGS. 13A-13C depict in vitro expression of RNA construct 23 (labeled “ERMA 23”) and nucleotide optimized variant 7 RNA construct (labeled “ERMA 23-7”) in HEK293T cells. FIG. 13A shows transfection rate as measured by percentage of total HEK293T population that is positive for presence of expressed protein. FIG. 13B shows total expression as measured by median fluorescence of the total HEK293T population for both transfected and non-transfected cells. FIG. 13C shows percentage of viable cells that are positive for presence of expressed protein, with non-transfected cells serving as a control.

FIGS. 14A-14B depict titers of antibodies elicited against P/CSP after immunization of mice with a pharmaceutical composition comprising RNA construct 23 (labeled “ERMA 23”) or variant 7 (labeled “ERMA 23-7”). FIG. 14A shows endpoint titers against full-length P/CSP on day 21, pre-boost. FIG. 14B shows endpoint titers against full-length P/CSP on day 35 after boost.

FIGS. 15A-15B depict epitope specificity of antibodies elicited upon immunization of mice with a pharmaceutical composition comprising RNA construct 23 (labeled “ERMA 23”) or variant 7 (labeled “ERMA 23-7”). FIG. 15A shows a diagram depicting localization of peptides using in multiplex assay in central region of P/CSP sequence. FIG. 15B shows binding to epitopes by calculating AUC of 8-point dilution of immune serum samples (left bars (dots) are RNA construct 23 (labeled “ERMA 23”); rights bars (diagonal stripes) are variant 7 (labeled “ERMA 23-7”).

FIGS. 16A-16C depict pro-inflammatory response from T cells after immunization of mice with a pharmaceutical composition comprising RNA construct 23 (labeled “ERMA 23”) or variant 7 (labeled “ERMA 23-7”). FIG. 16A shows production of IFNγ in mouse splenocytes after immunization with a pharmaceutical composition comprising RNA construct 23 (labeled “ERMA 23”) or variant 7 (labeled “ERMA 23-7”) and stimulation with P/CSP peptides. FIG. 16B shows production of IFNγ in combination with IL-2 in mouse splenocytes after immunization with a pharmaceutical composition comprising RNA construct 23 (labeled “ERMA 23”) or variant 7 (labeled “ERMA 23-7”) and stimulation with P/CSP peptides. FIG. 16C shows production of IFNγ in combination with IL-2 and TNFα in mouse splenocytes after immunization with a pharmaceutical composition comprising RNA construct 23 (labeled “ERMA 23”) or variant 7 (labeled “ERMA 23-7”) and stimulation with P/CSP peptides.

FIGS. 17A-17B depict titers of antibodies elicited against P/CSP after immunization of mice with a pharmaceutical composition comprising variant 7 (labeled “ERMA 23-7”) or vehicle. FIG. 17A shows endpoint titers (reciprocal serum titer) against full-length P/CSP pre-boost on Day 21. FIG. 17B shows endpoint titers (reciprocal serum titer) against full-length PfCSP after boost on Day 35.

FIG. 18 depicts epitope specificity of antibodies elicited upon immunization of mice with a pharmaceutical composition comprising variant 7 (labeled “ERMA 23-7”) or vehicle.

FIG. 19 depicts T-cell induction following administration of a pharmaceutical composition comprising 1 μg variant 7 (labeled “ERMA 23-7”) or DMSO.

FIGS. 20A-20B depict binding specificity of antibodies generated from mice immunized with variant 7 (labeled “ERMA 23-7”) to CSP protein in Plasmodium falciparum sporozoite lysates. FIG. 20A shows binding between antibodies in the sera of immunized mice and CSP protein in the sporozoite (spz) lysates represented as area under the curve (AUC) created when plotting dilution steps versus luminescence signal. FIG. 20B shows binding of murine anti-CSP mAb3SP2 used as a positive control.

### CERTAIN DEFINITIONS

In general, terminology used herein is in accordance with its understood meaning in the art, unless clearly indicated otherwise. Explicit definitions of certain terms are provided below; meanings of these and other terms in particular instances throughout this specification will be clear to those skilled in the art from context.

For purposes of this disclosure, the chemical elements are identified in accordance with the Periodic Table of Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents each of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomeric, and geometric (or conformational) mixtures of provided compounds are within the scope of the disclosure. For example, in some cases, provided compounds show one or more stereoisomers of a compound, and unless otherwise indicated, represents each stereoisomer alone and/or as a mixture. Unless otherwise stated, all tautomeric forms of provided compounds are within the scope of the disclosure.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by 13C- or 14C-enriched carbon are within the scope of this disclosure.

About. The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Agent. As used herein, the term “agent,” may refer to a physical entity. In some embodiments, an agent may be characterized by a particular feature and/or effect. For example, as used herein, the term “therapeutic agent” refers to a physical entity has a therapeutic effect and/or elicits a desired biological and/or pharmacological effect. In some embodiments, an agent may be a compound, molecule, or entity of any chemical class including, for example, a small molecule, polypeptide, nucleic acid, saccharide, lipid, metal, or a combination or complex thereof.

Amino acid: In its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Antigen: The term “antigen”, as used herein, refers to an agent that elicits an immune response; and/or (ii) an agent that binds to a T cell receptor (e.g., when presented by an MHC molecule) or to an antibody.

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of, susceptibility to, severity of, stage of, etc. the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Combination therapy: As used herein, the term “combination therapy” refers to those situations in which a subject is simultaneously exposed to two or more therapeutic regimens (e.g., two or more therapeutic agents (e.g., two or more antibody agents)). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, administration of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.

Dosing regimen: Those skilled in the art will appreciate that the term “dosing regimen” (or “therapeutic regimen”) may be used to refer to a set of unit doses (typically more than one) that are administered individually to a subject, typically separated by periods of time. In some embodiments, a given therapeutic agent has a recommended dosing regimen, which may involve one or more doses.

Encode: As used herein, the term “encode” or “encoding” refers to sequence information of a first molecule that guides production of a second molecule having a defined sequence of nucleotides (e.g., a polyribonucleotide) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or an RNA molecule encodes a polypeptide if transcription and translation of RNA corresponding to that gene produces the polypeptide in a cell or other biological system. In some embodiments, a coding region of a polyribonucleotide encoding a target antigen refers to a coding strand, the nucleotide sequence of which is identical to the polyribonucleotide sequence of such a target antigen. In some embodiments, a coding region of a polyribonucleotide encoding a target antigen refers to a non-coding strand of such a target antigen, which may be used as a template for transcription of a gene or cDNA.

Expression: As used herein, the term “expression” of a nucleic acid sequence refers to the generation of a gene product from the nucleic acid sequence. In some embodiments, a gene product can be a transcript, e.g., a polyribonucleotide as provided herein. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

Homology: As used herein, the term “homology” or “homolog” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “homologous” to one another if their sequences are at least 15%, 20%, 25%, 30%, 35%, 40%, 45%, 50%, 55%, 60%, 65%, 70%, 75%, 80%, 85%, 90%, 95%, or 99% identical. In some embodiments, polynucleotide molecules (e.g., DNA molecules and/or RNA molecules) and/or polypeptide molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Identity: As used herein, the term “identity” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or between polypeptide molecules are considered to be “substantially identical” to one another if their sequences are at least 80%, 85%, 90%, 95%, 96%, 97%, 98%, or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Increased, Induced, or Reduced: As used herein, these terms or grammatically comparable comparative terms, indicate values that are relative to a comparable reference measurement. For example, in some embodiments, an assessed value achieved with a provided composition (e.g., a pharmaceutical composition) may be “increased” relative to that obtained with a comparable reference composition. Alternatively or additionally, in some embodiments, an assessed value achieved in a subject may be “increased” relative to that obtained in the same subject under different conditions (e.g., prior to or after an event; or presence or absence of an event such as administration of a composition (e.g., a pharmaceutical composition) as described herein, or in a different, comparable subject (e.g., in a comparable subject that differs from the subject of interest in prior exposure to a condition, e.g., absence of administration of a composition (e.g., a pharmaceutical composition) as described herein.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance. In some embodiments, the term “reduced” or equivalent terms refers to a reduction in the level of an assessed value by at least 5%, at least 10%, at least 20%, at least 50%, at least 75% or higher, as compared to a comparable reference. In some embodiments, the term “reduced” or equivalent terms refers to a complete or essentially complete inhibition, i.e., a reduction to zero or essentially to zero. In some embodiments, the term “increased” or “induced” refers to an increase in the level of an assessed value by at least 10%, at least 20%, at least 30%, at least 40%, at least 50%, at least 80%, at least 100%, at least 200%, at least 500%, or higher, as compared to a comparable reference.

In order: As used herein with reference to a polynucleotide or polyribonucleotide, “in order” refers to the order of features from 5′ to 3′ along the polynucleotide or polyribonucleotide. As used herein with reference to a polypeptide, “in order” refers to the order of features moving from the N-terminal-most of the features to the C-terminal-most of the features along the polypeptide. “In order” does not mean that no additional features can be present among the listed features. For example, if Features A, B, and C of a polynucleotide are described herein as being “in order, Feature A, Feature B, and Feature C,” this description does not exclude, e.g., Feature D being located between Features A and B.

In vitro transcription: The term “in vitro transcription” or “IVT” as used herein means that the transcription (i.e., the generation of RNA) is conducted in a cell-free manner. In other words, IVT does not use living/cultured cells but rather the transcription machinery extracted from cells (e.g., cell lysates or the isolated components thereof, including an RNA polymerase (e.g., T7, T3 or SP6 polymerase)).

Isolated: The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Lipid: As used herein, the terms “lipid” and “lipid-like material” are broadly defined as molecules which comprise one or more hydrophobic moieties or groups and optionally also one or more hydrophilic moieties or groups. Molecules comprising hydrophobic moieties and hydrophilic moieties are also typically denoted as amphiphiles.

RNA lipidnanoparticle: As used herein, the term “RNA lipid nanoparticle” refers to a nanoparticle comprising at least one lipid and RNA molecule(s), e.g., one or more polyribonucleotides as provided herein. In some embodiments, an RNA lipid nanoparticle comprises at least one cationic amino lipid. In some embodiments, an RNA lipid nanoparticle comprises at least one cationic amino lipid, at least one helper lipid, and at least one polymer-conjugated lipid (e.g., PEG-conjugated lipid). In various embodiments, RNA lipid nanoparticles as described herein can have an average size (e.g., Z-average) of about 100 nm to 1000 nm, or about 200 nm to 900 nm, or about 200 nm to 800 nm, or about 250 nm to about 700 nm. In some embodiments of the present disclosure, RNA lipid nanoparticles can have a particle size (e.g., Z-average) of about 30 nm to about 200 nm, or about 30 nm to about 150 nm, about 40 nm to about 150 nm, about 50 nm to about 150 nm, about 60 nm to about 130 nm, about 70 nm to about 110 nm, about 70 nm to about 100 nm, about 80 nm to about 100 nm, about 90 nm to about 100 nm, about 70 to about 90 nm, about 80 nm to about 90 nm, or about 70 nm to about 80 nm. In some embodiments, an average size of lipid nanoparticles is determined by measuring the average particle diameter. In some embodiments, RNA lipid nanoparticles may be prepared by mixing lipids with RNA molecules described herein.

Neutralization: As used herein, the term “neutralization” refers to an event in which binding agents such as antibodies bind to a biological active site of a parasite such as a receptor binding protein, thereby inhibiting the parasitic infection of cells. In some embodiments, the term “neutralization” refers to an event in which binding agents eliminate or significantly reduce ability of infecting cells.

Nucleic acid/Polynucleotide: As used herein, the term “nucleic acid” refers to a polymer of at least 10 nucleotides or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro), reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Pharmaceutically effective amount. The term “pharmaceutically effective amount” or “therapeutically effective amount” refers to the amount which achieves a desired reaction or a desired effect alone or together with further doses. In the case of the treatment of a particular disease (e.g., malaria), a desired reaction in some embodiments relates to inhibition of the course of the disease (e.g., malaria). In some embodiments, such inhibition may comprise slowing down the progress of a disease (e.g., malaria) and/or interrupting or reversing the progress of the disease (e.g., malaria). In some embodiments, a desired reaction in a treatment of a disease (e.g., malaria) may be or comprise delay or prevention of the onset of a disease (e.g., malaria) or a condition (e.g., a malaria associated condition). An effective amount of a composition (e.g., a pharmaceutical composition) described herein will depend, for example, on disease (e.g., malaria) or a condition (e.g., a malaria associated condition) to be treated, the severity of such a disease (e.g., malaria) or a condition (e.g., a malaria associated condition), individual parameters of the patient, including, e.g., age, physiological condition, size and weight, the duration of treatment, the type of an accompanying therapy (if present), the specific route of administration and similar factors. Accordingly, doses of a composition (e.g., a pharmaceutical composition) described herein may depend on various of such parameters. In the case that a reaction in a patient is insufficient with an initial dose, higher doses (or effectively higher doses achieved by a different, more localized route of administration) may be used.

Polypeptide: As used herein, the term “polypeptide” refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications comprise acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 35 or more amino acids; in some embodiments, a conserved region encompasses at least one stretch of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous amino acids. In some embodiments, a relevant polypeptide may comprise or consist of a fragment of a parent polypeptide. In some embodiments, a polypeptide is a Plasmodium polypeptide construct described herein. A Plasmodium polypeptide construct is a polypeptide that includes one or more Plasmodium proteins, or one or more portions thereof. In some embodiments, a Plasmodium polypeptide construct described herein includes at least one region of Plasmodium CSP or a portion thereof. In some embodiments, a Plasmodium polypeptide construct additionally includes one or more additional amino acid sequences, such as a secretory signal (e.g., a heterologous secretory signal), a transmembrane region (e.g., a heterologous transmembrane region), a helper antigen, a multimerization region, and/or a linker, as described herein.

Prevent: As used herein, the term “prevent” or “prevention” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time. In some embodiments, prevention refers to reducing the risk of developing clinical malaria.

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Ribonucleic acid (RNA) or Polyribonucleotide: As used herein, the term “ribonucleic acid,” “RNA,” or “polyribonucleotide” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments, an RNA is a mRNA. In some embodiments, where an RNA is a mRNA, a RNA typically comprises at its 3′ end a poly(A) region. In some embodiments, where an RNA is a mRNA, an RNA typically comprises at its 5′ end an art-recognized cap structure, e.g., for recognizing and attachment of a mRNA to a ribosome to initiate translation. In some embodiments, a RNA is a synthetic RNA. Synthetic RNAs include RNAs that are synthesized in vitro (e.g., by enzymatic synthesis methods and/or by chemical synthesis methods). In some embodiments, a polyribonucleotide encodes a polypeptide, which is preferably is a Plasmodium polypeptide construct.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Subject. As used herein, the term “subject” refers to an organism to be administered with a composition described herein, e.g., for experimental, diagnostic, prophylactic, and/or therapeutic purposes. Typical subjects include animals (e.g., mammals such as mice, rats, rabbits, non-human primates, domestic pets, etc.) and humans. In some embodiments, a subject is a human subject. In some embodiments, a subject is suffering from a disease, disorder, or condition (e.g., malaria and/or a malaria-associated condition). In some embodiments, a subject is susceptible to a disease, disorder, or condition (e.g., malaria and/or a malaria-associated condition). In some embodiments, a subject displays one or more symptoms or characteristics of a disease, disorder, or condition (e.g., malaria and/or a malaria-associated condition). In some embodiments, a subject displays one or more non-specific symptoms of a disease, disorder, or condition (e.g., malaria and/or a malaria-associated condition). In some embodiments, a subject does not display any symptom or characteristic of a disease, disorder, or condition (e.g., malaria and/or a malaria-associated condition). In some embodiments, a subject is someone with one or more features characteristic of susceptibility to or risk of a disease, disorder, or condition (e.g., malaria and/or a malaria-associated condition). In some embodiments, a subject is a patient. In some embodiments, a subject is an individual to whom diagnosis and/or therapy is and/or has been administered.

Suffering from: An individual who is “suffering from” a disease, disorder, and/or condition (e.g., malaria and/or a malaria-associated condition) has been diagnosed with and/or displays one or more symptoms of a disease, disorder, and/or condition.

Susceptible to: An individual who is “susceptible to” a disease, disorder, and/or condition (e.g., malaria and/or a malaria-associated condition) is one who has a higher risk of developing the disease, disorder, and/or condition (e.g., malaria and/or a malaria-associated condition) than does a member of the general public. In some embodiments, an individual who is susceptible to a disease, disorder and/or condition (e.g., malaria and/or a malaria-associated condition) may not have been diagnosed with the disease, disorder, and/or condition (e.g., malaria and/or a malaria-associated condition). In some embodiments, an individual who is susceptible to a disease, disorder, and/or condition (e.g., malaria and/or a malaria-associated condition) may exhibit symptoms of the disease, disorder, and/or condition (e.g., malaria and/or a malaria-associated condition). In some embodiments, an individual who is susceptible to a disease, disorder, and/or condition (e.g., malaria and/or a malaria-associated condition) may not exhibit symptoms of the disease, disorder, and/or condition (e.g., malaria and/or a malaria-associated condition). In some embodiments, an individual who is susceptible to a disease, disorder, and/or condition (e.g., malaria and/or a malaria-associated condition) will develop the disease, disorder, and/or condition (e.g., malaria and/or a malaria-associated condition). In some embodiments, an individual who is susceptible to a disease, disorder, and/or condition (e.g., malaria and/or a malaria-associated condition) will not develop the disease, disorder, and/or condition (e.g., malaria and/or a malaria-associated condition).

Therapy: The term “therapy” refers to an administration or delivery of an agent or intervention that has a therapeutic effect and/or elicits a desired biological and/or pharmacological effect (e.g., has been demonstrated to be statistically likely to have such effect when administered to a relevant population). In some embodiments, a therapeutic agent or therapy is any substance that can be used to alleviate, ameliorate, relieve, inhibit, prevent, delay onset of, reduce severity of, and/or reduce incidence of one or more symptoms or features of a disease, disorder, and/or condition (e.g., malaria and/or a malaria-associated condition). In some embodiments, a therapeutic agent or therapy is a medical intervention that can be performed to alleviate, relieve, inhibit, present, delay onset of, reduce severity of, and/or reduce incidence of one or more symptoms or features of a disease, disorder, and/or condition.

Treat As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition (e.g., malaria and/or a malaria-associated condition). Treatment may be administered to a subject who does not exhibit signs of a disease, disorder, and/or condition (e.g., malaria and/or a malaria-associated condition). In some embodiments, treatment may be administered to a subject who exhibits only early signs of the disease, disorder, and/or condition (e.g., malaria and/or a malaria-associated condition), for example for the purpose of decreasing the risk of developing pathology associated with the disease, disorder, and/or condition. In some embodiments, treatment may be administered to a subject at a later-stage of disease, disorder, and/or condition (e.g., malaria and/or a malaria-associated condition).

Variant: As used herein, the term “variant” refers to a molecule that shows significant structural (e.g., primary or secondary) identity with a reference molecule but differs structurally from the reference molecule. For example, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polyribonucleotide differs in nucleotide content, but encodes the same polypeptide as the reference molecule. In some embodiments, a variant polyribonucleotide is a codon optimized variant.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls. The section headings used herein are for organizational purposes only and are not to be construed as limiting the subject matter described in any way.

### DETAILED DESCRIPTION OF CERTAIN EMBODIMENTS

The present disclosure provides technologies (e.g., compositions, methods, etc.) for delivery of a Plasmodium circumsporozoite protein (CSP) antigen. In particular, the present disclosure provides polyribonucleotides encoding a full-length Plasmodium CSP polypeptide, where the portion encoding the N-terminal region of the Plasmodium CSP polypeptide has a defined nucleotide content.

The present disclosure identifies a problem with certain polyribonucleotides that encode a full-length Plasmodium CSP polypeptide, in that they are characterized by an irregular peak (e.g., having a shoulder or a double peak), for example, when analyzed by electrophoresis (e.g., capillary electrophoresis, e.g., using a fragment analyzer). The present disclosure provides an unexpected solution to this problem: the irregular peak was resolved by specifically altering the nucleotide content of the portion encoding the N-terminal region of the Plasmodium CSP polypeptide. In some embodiments, the present disclosure provides optimized polyribonucleotides that encode a full-length Plasmodium CSP polypeptide whose integrity can be confirmed using capillary electrophoresis (e.g., a fragment analyzer). The present disclosure encompasses a recognition that such polyribonucleotides and compositions including the same may be useful for treatment and/or prevention of malaria.

Malaria is a mosquito-borne infectious disease caused by single-celled eukaryotic Plasmodium parasites that are transmitted by the bite of Anopheles spp. mosquitoes (Phillips, M., et al. Malaria. Nat Rev Dis Primers 3, 17050, 2017, which is incorporated herein by reference in its entirety). Mosquitoes that transmit malaria must have been infected through a previous blood meal taken from an infected subject (e.g., a human). When a mosquito bites an infected subject a small amount of blood is taken in containing Plasmodium parasites. The infected mosquito can then subsequently bite a non-infected subject, infecting the subject.

Malaria remains one of the most serious infectious diseases, causing approximately 200 million clinical cases and 500,000-600,000 deaths annually. Although significant effort has been invested in developing therapeutic treatments for malaria, many Plasmodium parasites have developed resistance to available therapeutics. According to Malaria Eradication Research Agenda Initiative, malaria eradication will only be achievable through effective vaccination. An effective malaria vaccine remains an unmet medical need of critical importance for global health.

### I. Circumsporozoite Protein (CSP)

Circumsporozoite protein (CSP) is a multifunctional protein that is involved in Plasmodium life cycle, as it is required for the formation of sporozoites in the mosquito midgut, the release of sporozoites from the oocyst, invasion of salivary glands, attachment of sporozoites to hepatocytes in the liver, and sporozoite invasion of hepatocytes (see, e.g., Zhao et al. (2016) PLoS ONE 11(8): e0161607, which is incorporated herein by reference in its entirety). CSP is present in all Plasmodium species, and although variation exists in the amino acid sequence across species, the overall domain structure of a central repeat region and nonrepeat flanking regions is well conserved (see, e.g., Zhao et al. (2016) PLoS ONE 11(8): e0161607; Wahl et al. (2022) J. Exp. Med. 219: e20201313, each of which is incorporated herein by reference in its entirety). CSP sequences are known (see, e.g., UniProt accession numbers A0A2L1CF52, A0A2L,1CF88, C6FGZ3, C6FH2,7 C6FHG7, M1V060, M1V0A3, M1V0B0, M1VOC4, M1VOE0, M1V9I4, M1VFN9, M1VKZ2, P02893, Q5EIJ9, Q5EIK2, Q5EIK8, Q5EIL3, Q5EIL5, Q5EIL8, Q5R2L2, Q7K740, Q89G5, Q813, Q814), and Table 1 includes exemplary sequences for CSP P. falciparum isolates from Asia, South America and Africa.

TABLE 1
Exemplary CSP P. falciparum isolates from Asia, South America and Africa

| Accession number | Country | References |
|---|---|---|
| AB121015.1 | 7G8 (Brazil) | Tanabe, K., et al., Genetic distance in housekeeping genes between |
| | | Plasmodium falciparum and Plasmodium reichenowi and within P. |
| | | falciparum. J. Mol. Evol. 59 (5), 687-694 (2004), which is |
| | | incorporated herein by reference. |
| AB121017.1 | Dd2 (Indochina) | Tanabe (2004) |
| AB121018.1 | HB3 (Honduras) | Tanabe (2004) |
| AB121020.1 | MAD20 (PNG) | Tanabe (2004) |
| AB121021.1 | RO-33 (Ghana) | Tanabe (2004) |
| AJ269946.1 | K1 (Thailand) | de Stricker, K., et al., Sequence variation in the non-repeat region of |
| | | the Plasmodium falciparum glutamate rich protein (GLURP) from |
| | | Brazil, Senegalese and Burmese field isolates and from laboratory |
| | | strains. (Unpublished), which is incorporated herein by reference. |
| M15505.1 | Wellcome (West | Lockyer M J, Schwarz R T. Strain variation in the circumsporozoite |
| | African) | protein gene of Plasmodium falciparum. Mol Biochem Parasitol. |
| | | 2; 22(1): 101-8 (1987), which is incorporated herein by reference. |
| M22982.1 | NF54 | Caspers P, Gentz R, Matile H, Pink J R, Sinigaglia FThe |
| | (Netherlands) | circumsporozoite protein gene from NF54, a Plasmodium falciparum |
| | | isolate used in malaria vaccine trials. Mol Biochem |
| | | Parasitol. 35(2): 185-9. (1989), which is incorporated herein by |
| | | reference. |
| AF540441.1, | India | Escalante A A, Grebert H M, Isea R, Goldman I F, Basco L, Magris |
| AF540442.1, | | M, Biswas S, Kariuki S and Lal A A. A study of genetic diversity in the |
| AF540472.1, | | gene encoding the circumsporozoite protein (CSP) of Plasmodium |
| AF540481.1 to | | falciparum from different transmission areas--XVI. Asembo Bay |
| AF540488.1 | | Cohort Project. Mol Biochem Parasitol125(1-2): 83-90 (2002), which |
| | | is incorporated herein by reference. |
| AJ269943.1 | India | de Stricker, K. (Unpublished) |
| DQ521663.1 to | Iran | Zakeri S, et al., Restricted T-cell epitope diversity in the |
| DQ521752.1 | | circumsporozoite protein from Plasmodium falciparum populations |
| | | prevalent in Iran. .Am J Trop Med Hyg.; 76(6): 1046-51 (2007), which |
| | | is incorporated herein by reference. |
| AJ269945.1, | Myanmar | de Stricker, K (Unpublished) |
| AJ269955.1- | | |
| AJ269960.1 | | |
| DQ193595.1 | Myanmar | Jalloh A, van Thien H, Ferreira M U, Ohashi J, Matsuoka H, Kanbe |
| | | T, Kikuchi A, Kawamoto F. Sequence variation in the T-cell epitopes |
| | | of the Plasmodium falciparum circumsporozoite protein among field |
| | | isolates is temporally stable: a 5-year longitudinal study in southern |
| | | Vietnam. J Clin Microbiol 44(4): 1229-35 (2006), which is incorporated |
| | | herein by reference in its entirety. |
| FJ232142.1- | Thailand | Putaporntip C, Jongwutiwes S, Hughes A L. Natural selection |
| FJ232166.1, | | maintains a stable polymorphism at the circumsporozoite protein |
| FJ232168.1- | | locus of Plasmodium falciparum in a low endemic area. Infect Genet |
| FJ232364.1 | | Evol. 9(4): 567-73 (2009), which is incorporated herein by reference |
| | | in its entirety. |
| GQ890702.1- | Thailand | Jongwutiwes S, Putaporntip C, Hughes A L. Bottleneck effects on |
| GQ890789.1 | | vaccine-candidate antigen diversity of malaria parasites in Thailand. |
| | | Vaccine. 19; 28(18): 3112-7. (2010), which is incorporated herein by |
| | | reference in its entirety. |
| M19752.1 | Thailand | del Portillo H A, Nussenzweig R S, Enea V. Circumsporozoite gene of a |
| | | Plasmodium falciparum strain from Thailand. Mol Biochem |
| | | Parasitol. 24(3): 289-94. (1987), which is incorporated herein by |
| | | reference in its entirety. |
| M83149.1, | Thailand | Jongwutiwes S, Tanabe K, Hughes M K, Kanbara H, Hughes A L. Allelic |
| M83150.1- | | variation in the circumsporozoite protein of Plasmodium falciparum |
| M83174.1 | | from Thai field isolates. Am J Trop Med Hyg; 51(5): 659-68 (1994), |
| | | which is incorporated herein by reference in its entirety. |
| DQ193573.1- | Vietnam | Jalloh (2006) |
| DQ193594.1 | | |
| DQ193593.1- | Indonesia | Jalloh (2006) |
| DQ193594.1 | | |
| AB116602.1- | Vanuatu | Tanabe (2004) |
| AB116607.1 | | |
| AF540458.1- | Venezuela | Escalante (2002). |
| AF540460.1, | | |
| AF540464.1, | | |
| AF540466.1, | | |
| AF540469.1- | | |
| AF540471.1, | | |
| AF540478.1- | | |
| AF540479.1 | | |
| AJ269941.1, | Brazil | de Stricker (Unpublished) |
| AJ269971.1- | | |
| AJ269978.1 | | |
| K02194.1 | Brazil | Dame J B, Williams J L et al. Structure of the gene encoding the |
| | | immunodominant surface antigen on the sporozoite of the human |
| | | malaria parasite Plasmodium falciparum. Science. 10; 225(4662): 593- |
| | | 1984), which is incorporated herein by reference in its entirety. |
| AF540443.1, | Asembo Bay | Escalante (2002). |
| AF540447.1- | | |
| AF540448.1, | | |
| AF540450.1- | | |
| AF540451.1, | | |
| AF540455.1- | | |
| AF540457.1, | | |
| AF540461.1- | | |
| AF540463.1, | | |
| AF540465.1, | | |
| AF540467.1, | | |
| AF540473.1 = | | |
| AF540477.1 | | |
| AF181833.1- | Gambia | Alloueche A, Silveira H, Conway D J, Bojang K, Doherty T, Cohen |
| AF181835.1, | | J, Pinder M, Greenwood B M. High-throughput sequence typing of T- |
| AY878598.1- | | cell epitope polymorphisms in Plasmodium falciparum |
| AY878641.1 | | circumsporozoite protein. Mol Biochem Parasitol. 5; 106(2): 273-82 |
| | | (2000), which is incorporated herein by reference in its entirety. |
| AY878598.1 | Gambia | Weedall G D, Preston B M, Thomas A W, Sutherland C J, Conway D J. |
| | | Differential evidence of natural selection on two leading sporozoite |
| | | stage malaria vaccine candidate antigens. Int J Parasitol. 37(1): 77- |
| | | 85 (2007), which is incorporated herein by reference in its entirety. |
| AJ269948.1, | Senegal | de Stricker (Unpublished) |
| AJ269961.1- | | |
| AJ269970.1 | | |
| GQ119637.1- | Sierra Leone | Jalloh A, Jalloh M, Matsuoka H. T-cell epitope polymorphisms of the |
| GQ119678.1 | | Plasmodium falciparum circumsporozoite protein among field isolates |
| | | from Sierra Leone: age-dependent haplotype distribution. Malar J. |
| | | 5; 8: 120. (2009), which is incorporated herein by reference in its |
| | | entirety. |
| AF540444.1- | Cameroon | Escalante (2002). |
| AF540446.1, | | |
| AF540449.1, | | |
| AF540452.1- | | |
| AF540454.1, | | |
| AF540468.1, | | |
| AF540480.1 | | |

An exemplary wild-type CSP polypeptide amino sequence from Plasmodium falciparum isolate 3D7 is presented in Table 2 as SEQ ID NO: 1, and includes the following: a secretory signal (amino acids 1-18); an N-terminal domain (amino acids 19-104); a junction region (amino acids 93-104), a central domain (amino acids 105-272); and a C-terminal domain (amino acids 273-397). In exemplary SEQ ID NO: 1, the N-terminal domain includes an N-terminal region (amino acids 19-80); an N-terminal end region (amino acids 81-92); and a junction region (amino acids 93-104). In exemplary SEQ ID NO: 1, the junction region includes an R1 region (amino acids 93-97) and a junction (SEQ ID NO: 22) at positions 98-104. In exemplary SEQ ID NO: 1, the central domain includes a minor repeat region (amino acids 105-128) and a major repeat region (amino acids 129-272). In exemplary SEQ ID NO: 1, the minor repeat region includes three repeats of the amino acid sequence NANPNVDP (SEQ ID NO: 29). In exemplary SEQ ID NO: 1, the major repeat region includes 35 repeats of the amino acid sequence NANP (SEQ ID NO: 33), wherein 35 repeats of the amino acid sequence NANP (SEQ ID NO: 33) are separated into two contiguous stretches, and wherein one stretch includes 17 repeats of the amino acid sequence NANP (SEQ ID NO: 33) and one includes 18 repeats of the amino acid sequence NANP (SEQ ID NO: 33) which flank an amino acid sequence of NVDP (SEQ ID NO: 34). The major repeat region includes the amino acid sequences NPNANP (SEQ ID NO: 35) and NANPNA (SEQ ID NO: 36). In exemplary SEQ ID NO: 1, the C-terminal domain includes a C-terminal region (amino acids 273-375), a serine-valine (amino acids 376-377), and a transmembrane domain (amino acids 378-397). In exemplary SEQ ID NO: 1, the C-terminal region includes a Th2R region (amino acids 314-327) and a Th3R region (amino acids 352-363). Exemplary CSP amino acid sequence is provided in Table 2.

TABLE 2
Exemplary CSP sequences

| SEQ ID | | |
|---|---|---|
| NO: | Description | Sequence |
| 1 | CSP | MMRKLAILSVSSFLFVEALFQEYQCYGSSSNTRVLNELNYDNAGTNLYNELEMNYY |
| | Amino Acid | GKQENWYSLKKNSRSLGENDDGNNEDNEKLRKPKHKKLKQPADGNPDPNANPNVDP |
| | | NANPNVDPNANPNVDPNANPNANPNANPNANPNANPNANPNANPNANPNANPNANP |
| | | NANPNANPNANPNANPNANPNANPNANPNVDPNANPNANPNANPNANPNANPNANP |
| | | NANPNANPNANPNANPNANPNANPNANPNANPNANPNANPNANPNANPNKNNQGNG |
| | | QGHNMPNDPNRNVDENANANSAVKNNNNEEPSDKHIKEYLNKIONSLSTEWSPCSV |
| | | TCGNGIQVRIKPGSANKPKDELDYANDIEKKICKMEKCSSVFNVVNSSIGLIMVLS |
| | | FLFLN |
| 2 | CSP | ATGATGAGAAAATTAGCTATTTTATCTGTTTCTTCCTTTTTATTTGTTGAGGCCTT |
| | DNA | ATTCCAGGAATACCAGTGCTATGGAAGTTCGTCAAACACAAGGGTTCTAAATGAAT |
| | | TAAATTATGATAATGCAGGCACTAATTTATATAATGAATTAGAAATGAATTATTAT |
| | | GGGAAACAGGAAAATTGGTATAGTCTTAAAAAAAATAGTAGATCACTTGGAGAAAA |
| | | TGATGATGGAAATAACGAAGACAACGAGAAATTAAGGAAACCAAAACATAAAAAAT |
| | | TAAAGCAACCAGCGGATGGTAATCCTGATCCAAATGCAAACCCAAATGTAGATCCC |
| | | AATGCCAACCCAAATGTAGATCCAAATGCAAACCCAAATGTAGATCCAAATGCAAA |
| | | CCCAAATGCAAACCCAAATGCAAACCCAAATGCAAACCCAAATGCAAACCCAAATG |
| | | CAAACCCAAATGCAAACCCAAATGCAAACCCAAATGCAAACCCAAATGCAAACCCA |
| | | AATGCAAACCCAAATGCAAACCCAAATGCAAACCCAAATGCAAACCCCAATGCAAA |
| | | TCCTAATGCAAACCCAAATGCAAACCCAAACGTAGATCCTAATGCAAATCCAAATG |
| | | CAAACCCAAACGCAAACCCCAATGCAAATCCTAATGCAAACCCCAATGCAAATCCT |
| | | AATGCAAATCCTAATGCCAATCCAAATGCAAATCCAAATGCAAACCCAAACGCAAA |
| | | CCCCAATGCAAATCCTAATGCCAATCCAAATGCAAATCCAAATGCAAACCCAAATG |
| | | CAAACCCAAATGCAAACCCCAATGCAAATCCTAATAAAAACAATCAAGGTAATGGA |
| | | CAAGGTCACAATATGCCAAATGACCCAAACCGAAATGTAGATGAAAATGCTAATGC |
| | | CAACAGTGCTGTAAAAAATAATAATAACGAAGAACCAAGTGATAAGCACATAAAAG |
| | | AATATTTAAACAAAATACAAAATTCTCTTTCAACTGAATGGTCCCCATGTAGTGTA |
| | | ACTTGTGGAAATGGTATTCAAGTTAGAATAAAGCCTGGCTCTGCTAATAAACCTAA |
| | | AGACGAATTAGATTATGCAAATGATATTGAAAAAAAAATTTGTAAAATGGAAAAAT |
| | | GTTCCAGTGTGTTTAATGTCGTAAATAGTTCAATAGGATTAATAATGGTATTATCC |
| | | TTCTTGTTCCTTAATTAG |
| 3 | CSP | AUGAUGAGAAAAUUAGCUAUUUUAUCUGUUUCUUCCUUUUUAUUUGUUGAGGCCUU |
| | RNA | AUUCCAGGAAUACCAGUGCUAUGGAAGUUCGUCAAACACAAGGGUUCUAAAUGAAU |
| | | UAAAUUAUGAUAAUGCAGGCACUAAUUUAUAUAAUGAAUUAGAAAUGAAUUAUUAU |
| | | GGGAAACAGGAAAAUUGGUAUAGUCUUAAAAAAAAUAGUAGAUCACUUGGAGAAAA |
| | | UGAUGAUGGAAAUAACGAAGACAACGAGAAAUUAAGGAAACCAAAACAUAAAAAAU |
| | | UAAAGCAACCAGCGGAUGGUAAUCCUGAUCCAAAUGCAAACCCAAAUGUAGAUCCC |
| | | AAUGCCAACCCAAAUGUAGAUCCAAAUGCAAACCCAAAUGUAGAUCCAAAUGCAAA |
| | | CCCAAAUGCAAACCCAAAUGCAAACCCAAAUGCAAACCCAAAUGCAAACCCAAAUG |
| | | CAAACCCAAAUGCAAACCCAAAUGCAAACCCAAAUGCAAACCCAAAUGCAAACCCA |
| | | AAUGCAAACCCAAAUGCAAACCCAAAUGCAAACCCAAAUGCAAACCCCAAUGCAAA |
| | | UCCUAAUGCAAACCCAAAUGCAAACCCAAACGUAGAUCCUAAUGCAAAUCCAAAUG |
| | | CAAACCCAAACGCAAACCCCAAUGCAAAUCCUAAUGCAAACCCCAAUGCAAAUCCU |
| | | AAUGCAAAUCCUAAUGCCAAUCCAAAUGCAAAUCCAAAUGCAAACCCAAACGCAAA |
| | | CCCCAAUGCAAAUCCUAAUGCCAAUCCAAAUGCAAAUCCAAAUGCAAACCCAAAUG |
| | | CAAACCCAAAUGCAAACCCCAAUGCAAAUCCUAAUAAAAACAAUCAAGGUAAUGGA |
| | | CAAGGUCACAAUAUGCCAAAUGACCCAAACCGAAAUGUAGAUGAAAAUGCUAAUGC |
| | | CAACAGUGCUGUAAAAAAUAAUAAUAACGAAGAACCAAGUGAUAAGCACAUAAAAG |
| | | AAUAUUUAAACAAAAUACAAAAUUCUCUUUCAACUGAAUGGUCCCCAUGUAGUGUA |
| | | ACUUGUGGAAAUGGUAUUCAAGUUAGAAUAAAGCCUGGCUCUGCUAAUAAACCUAA |
| | | AGACGAAUUAGAUUAUGCAAAUGAUAUUGAAAAAAAAAUUUGUAAAAUGGAAAAAU |
| | | GUUCCAGUGUGUUUAAUGUCGUAAAUAGUUCAAUAGGAUUAAUAAUGGUAUUAUCC |
| | | UUCUUGUUCCUUAAUUAG |

### A. Full-Length CSP Polypeptides

The present disclosure, among other things, utilizes RNA technologies as a modality to express a full-length Plasmodium CSP polypeptide. In some embodiments, a full-length Plasmodium CSP polypeptide described herein includes one or more regions or portions of CSP from Plasmodium falciparum, preferably from Plasmodium falciparum isolate 3D7. In some embodiments, a full-length Plasmodium CSP polypeptide described herein includes one or more of a N-terminal region, a N-terminal end region, a junction region, a minor repeat region, a major repeat region and a C-terminal region or corresponding portions thereof of CSP from Plasmodium falciparum, preferably from Plasmodium falciparum isolate 3D7.

In some embodiments, a full-length Plasmodium CSP polypeptide comprises (i) a secretory signal, (ii) a Plasmodium CSP N-terminal domain, where the Plasmodium CSP N-terminal domain comprises an N-terminal region, an N-terminal end region, and a junction region, (iii) a Plasmodium CSP central domain comprising a minor repeat region and a major repeat region, and (iv) a Plasmodium CSP C-terminal domain comprising a C-terminal region and a transmembrane region.

In some embodiments, a full-length Plasmodium CSP polypeptide described herein has the structure: N-terminal region-N-terminal end region-junction region-minor repeat region-major repeat region-C-terminal region, where the regions are from CSP from Plasmodium falciparum, preferably from Plasmodium falciparum isolate 3D7. In some embodiments, such a full-length Plasmodium CSP polypeptide has immediately following the C-terminal region a serine or a serine and a valine.

In some embodiments, an N-terminal region comprises an amino acid sequence of positions 19 to 80 of SEQ ID NO: 1, or an amino acid sequence having at least 99%, 98%, 97%, 96%, or 95% identity to the amino acid sequence of positions 19 to 80 of SEQ ID NO: 1. In some embodiments, an N-terminal region comprises an amino acid sequence having at least 99%, 98%, 97%, 96%, or 95% identity to SEQ ID NO: 18. In some embodiments, an N-terminal region comprises or consists of an amino acid sequence of SEQ ID NO: 18.

In some embodiments, an N-terminal end region comprises an amino acid sequence of positions 81 to 92 of SEQ ID NO: 1, or an amino acid sequence of positions 81 to 92 of SEQ ID NO:1 having 1 or 2 amino acid substitutions. In some embodiments, an N-terminal end region comprises an amino acid sequence of SEQ ID NO: 19 having 1 or 2 amino acid substitutions. In some embodiments, an N-terminal end region comprises or consists of an amino acid sequence of SEQ ID NO: 19.

In some embodiments, a junction region comprises an amino acid sequence of positions 93 to 104 of SEQ ID NO:1, or an amino acid sequence of positions 93 to 104 of SEQ ID NO: 1 having 1 or 2 amino acid substitutions. In some embodiments, a junction region comprises an amino acid sequence of SEQ ID NO: 20 having 1 or 2 amino acid substitutions. In some embodiments, a junction region comprises or consists of an amino acid sequence of SEQ ID NO: 20.

In some embodiments, an N-terminal domain comprises an amino acid sequence of positions 19 to 104 of SEQ ID NO: 1, or an amino acid sequence having at least 99%, 98%, 97%, 96%, or 95% identity to the amino acid sequence of positions 19 to 104 of SEQ ID NO: 1. In some embodiments, an N-terminal domain comprises an amino acid sequence having at least 99%, 98%, 97%, 96%, or 95% identity to SEQ ID NO: 7. In some embodiments, an N-terminal domain comprises or consists of an amino acid sequence of SEQ ID NO: 7.

In some embodiments, a minor repeat region comprises an amino acid sequence of positions 105 to 128 of SEQ ID NO: 1, or an amino acid sequence having at least 99%, 98%, 97%, 96%, or 95% identity to the amino acid sequence of positions 105 to 128 of SEQ ID NO: 1. In some embodiments, a minor repeat region comprises an amino acid sequence having at least 99%, 98%, 97%, 96%, or 95% identity to SEQ ID NO: 26. In some embodiments, a minor repeat region comprises or consists of an amino acid sequence of SEQ ID NO: 26.

In some embodiments, a major repeat region comprises an amino acid sequence of positions 129 to 272 of SEQ ID NO: 1, or an amino acid sequence having at least 99%, 98%, 97%, 96%, or 95% identity to the amino acid sequence of positions 129 to 272 of SEQ ID NO:1. In some embodiments, a major repeat region comprises an amino acid sequence having at least 99%, 98%, 97%, 96%, or 95% identity to SEQ ID NO: 30. In some embodiments, a major repeat region comprises or consists of an amino acid sequence of SEQ ID NO: 30.

In some embodiments, a central domain comprises an amino acid sequence of positions 105 to 272 of SEQ ID NO: 1, or an amino acid sequence having at least 99%, 98%, 97%, 96%, or 95% identity to the amino acid sequence of positions 105 to 272 of SEQ ID NO: 1. In some embodiments, a central domain comprises an amino acid sequence having at least 99%, 98%, 97%, 96%, or 95% identity to SEQ ID NO: 23. In some embodiments, a central domain comprises or consists of an amino acid sequence of SEQ ID NO: 23.

In some embodiments, a C-terminal region comprises an amino acid sequence of positions 273 to 373 of SEQ ID NO: 1, or an amino acid sequence having at least 99%, 98%, 97%, 96%, 95%, 90%, 85%, or 80% identity to the amino acid sequence of positions 273 to 373 of SEQ ID NO: 1. In some embodiments, a C-terminal region comprises an amino acid sequence having at least 99%, 98%, 97%, 96%, or 95% identity to SEQ ID NO: 40. In some embodiments, a C-terminal region comprises or consists of an amino acid sequence of SEQ ID NO: 40.

In some embodiments, a full-length Plasmodium CSP polypeptide construct described herein includes a transmembrane region. In some embodiments, a transmembrane region comprises or consists of a Plasmodium transmembrane region. In some embodiments, a utilized transmembrane region is one that is normally associated with CSP in nature. In some embodiments, a Plasmodium transmembrane region comprises or consists of a Plasmodium CSP glycosylphosphatidylinositol (GPI) anchor region. In some embodiments, a Plasmodium CSP GPI anchor region is from Plasmodium falciparum. In some embodiments, a Plasmodium CSP GPI anchor region is from Plasmodium falciparum isolate 3D7.

In some embodiments, a Plasmodium CSP GPI anchor region comprises an amino acid sequence of amino acids 374 to 397 of SEQ ID NO: 1, or an amino acid sequence of positions 374 to 397 of SEQ ID NO:1 having 1 or 2 amino acid substitutions. In some embodiments, a Plasmodium CSP GPI anchor region comprises an amino acid sequence of SEQ ID NO: 43 having 1 or 2 amino acid substitutions. In some embodiments, a Plasmodium CSP GPI anchor region comprises or consists of an amino acid sequence: FNWNSSIGLIMVLSFLFLN (SEQ ID NO: 43).

In some embodiments, a C-terminal domain comprises an amino acid sequence of positions 273 to 397 of SEQ ID NO: 1, or an amino acid sequence having at least 99%, 98%, 97%, 96%, 95%, 90%, 85%, or 80% identity to the amino acid sequence of positions 273 to 397 of SEQ ID NO: 1. In some embodiments, a C-terminal domain comprises an amino acid sequence having at least 99%, 98%, 97%, 96%, or 95% identity to SEQ ID NO: 37. In some embodiments, a C-terminal region comprises or consists of an amino acid sequence of SEQ ID NO: 37.

In some embodiments, a full-length Plasmodium CSP polypeptide construct comprises an amino acid sequence having at least 99%, 98%, 97%, 96%, or 95% identity to the amino acid sequence of positions 19-375 of SEQ ID NO: 1. In some embodiments, a full-length Plasmodium CSP polypeptide construct comprises or consists of an amino acid sequence of positions 19-375 of SEQ ID NO: 1.

In some embodiments, a full-length Plasmodium CSP polypeptide construct described herein includes a Plasmodium secretory signal. In some embodiments, a Plasmodium secretory signal comprises or consists of a Plasmodium CSP secretory signal. In some embodiments, a Plasmodium CSP secretory signal is from Plasmodium falciparum. In some embodiments, a Plasmodium CSP secretory signal is from Plasmodium falciparum isolate 3D7. In some embodiments, a Plasmodium CSP secretory signal comprises amino acids 1 to 18 of SEQ ID NO: 1, or an amino acid sequence of positions 1 to 18 of SEQ ID NO:1 having 1 or 2 amino acid substitutions. In some embodiments, a Plasmodium CSP secretory signal comprises an amino acid sequence of SEQ ID NO: 4 having 1 or 2 amino acid substitutions. In some embodiments, a Plasmodium CSP secretory signal comprises or consists of an amino acid sequence of SEQ ID NO: 4.

In some embodiments, a full-length Plasmodium CSP polypeptide construct comprises an amino acid sequence having at least 99%, 98%, 97%, 96%, or 95% identity to the amino acid sequence of positions 1-397 of SEQ ID NO: 1. In some embodiments, a full-length Plasmodium CSP polypeptide construct comprises or consists of an amino acid sequence of positions 1-397 of SEQ ID NO: 1.

### B. Encoding Polyribonucleotides

The present disclosure provides, among other things, polynucleotides encoding a full-length Plasmodium CSP polypeptide or portion thereof. The present disclosure identifies a problem with certain polyribonucleotides that encode a full-length Plasmodium CSP polypeptide, in that they are characterized by an irregular peak (e.g., having a shoulder or a double peak), for example, when analyzed by electrophoresis (e.g., capillary electrophoresis, e.g., using a fragment analyzer). The present disclosure encompasses a recognition that such an irregular peak is inconsistent with standards of good manufacturing practices. For example, in some embodiments, RNA integrity cannot be confirmed if there is an irregular peak. In some embodiments, an irregular peak may disguise RNA degradation and/or a contaminant. The present disclosure provides polyribonucleotides that encode a full-length Plasmodium CSP polypeptide having certain characteristics that address this problem.

The present disclosure provides the insight that the nucleotide content of a portion of a polyribonucleotide encoding, e.g., the N-terminal domain of the Plasmodium CSP polypeptide can affect peak shape. In some embodiments, the present disclosure provides polyribonucleotides that encode a full-length Plasmodium CSP polypeptide, where nucleotide content of the portion encoding the N-terminal domain of the Plasmodium CSP polypeptide is within defined ranges.

In some embodiments, the present disclosure provides polyribonucleotides comprising a coding sequence that encodes a full-length Plasmodium CSP polypeptide, where the full-length Plasmodium CSP polypeptide comprises a Plasmodium CSP N-terminal domain, and where the coding sequence has an adenine content that is between 35% and 42%, and the portion of the coding sequence that encodes the Plasmodium CSP N-terminal domain has an adenine content that is at least 35%.

In some embodiments, the portion of the coding sequence that encodes the Plasmodium CSP N-terminal domain has an adenine content that is between 35% and 45%. In some embodiments, the portion of the coding sequence that encodes the Plasmodium CSP N-terminal domain has an adenine content that is between 36% and 42%. In some embodiments, the coding sequence has an adenine content that is between that is between 35% and 36.5%.

In some embodiments, the coding sequence has a uracil content that is between 14% and 20%, and the portion of the coding sequence that encodes the Plasmodium CSP N-terminal domain has a uracil content that is at least 10%. In some embodiments, the coding sequence has a guanine content that is between 15% and 19.5%, and the portion of the coding sequence encoding a Plasmodium CSP N-terminal domain has a guanine content that is less than 27%. In some embodiments, the coding sequence has a cytosine content that is between 22% and 31%, and the portion of the coding sequence that encodes the Plasmodium CSP N-terminal domain has a cytosine content that is less than 28%.

In some embodiments, the present disclosure provides polyribonucleotides comprising a coding sequence that encodes a full-length Plasmodium CSP polypeptide, where the full-length Plasmodium CSP polypeptide comprises a Plasmodium CSP N-terminal domain, and where the coding sequence has a uracil content that is between 14% and 20%, and the portion of the coding sequence that encodes the Plasmodium CSP N-terminal domain has a uracil content that is at least 10%.

In some embodiments, the portion of the coding sequence that encodes the Plasmodium CSP N-terminal domain has a uracil content that is between 12% and 25%. In some embodiments, the portion of the coding sequence that encodes the Plasmodium CSP N-terminal domain has a uracil content that is between 17% and 22%. In some embodiments, the coding sequence has a uracil content that is between that is between 15% and 16.5%.

In some embodiments, the coding sequence has an adenine content that is between 35% and 42%, and the portion of the coding sequence that encodes the Plasmodium CSP N-terminal domain has an adenine content that is at least 35%. In some embodiments, the coding sequence has a guanine content that is between 15% and 19.5%, and the portion of the coding sequence encoding a Plasmodium CSP N-terminal domain has a guanine content that is less than 27%. In some embodiments, the coding sequence has a cytosine content that is between 22% and 31%, and the portion of the coding sequence that encodes the Plasmodium CSP N-terminal domain has a cytosine content that is less than 28%.

In some embodiments, the present disclosure provides polyribonucleotides comprising a coding sequence that encodes a full-length Plasmodium CSP polypeptide, where the full-length Plasmodium CSP polypeptide comprises a Plasmodium CSP N-terminal domain, and where the coding sequence has a guanine content that is between 15% and 19.5%, and the portion of the coding sequence encoding a Plasmodium CSP N-terminal domain has a guanine content that is less than 27%.

In some embodiments, the portion of the coding sequence that encodes the Plasmodium CSP N-terminal domain has a guanine content that is between 19% and 26%. In some embodiments, the portion of the coding sequence that encodes the Plasmodium CSP N-terminal domain has a guanine content that is between 19.5% and 24.5%. In some embodiments, the coding sequence has a guanine content that is between that is between 18.5% and 19.5%.

In some embodiments, the coding sequence has an adenine content that is between 35% and 42%, and the portion of the coding sequence that encodes the Plasmodium CSP N-terminal domain has an adenine content that is at least 35%. In some embodiments, the coding sequence has a uracil content that is between 14% and 20%, and the portion of the coding sequence that encodes the Plasmodium CSP N-terminal domain has a uracil content that is at least 10%. In some embodiments, the coding sequence has a cytosine content that is between 22% and 31%, and the portion of the coding sequence that encodes the Plasmodium CSP N-terminal domain has a cytosine content that is less than 28%.

In some embodiments, the present disclosure provides polyribonucleotides comprising a coding sequence that encodes a full-length Plasmodium CSP polypeptide, where the full-length Plasmodium CSP polypeptide comprises a Plasmodium CSP N-terminal domain, and where the coding sequence has a cytosine content that is between 22% and 31%, and the portion of the coding sequence that encodes the Plasmodium CSP N-terminal domain has a cytosine content that is less than 28%.

In some embodiments, the portion of the coding sequence that encodes the Plasmodium CSP N-terminal domain has a cytosine content that is between 12% and 27% cytosine. In some embodiments, the portion of the coding sequence that encodes the Plasmodium CSP N-terminal domain has a cytosine content that is between 15% and 22% cytosine. In some embodiments, the coding sequence has a cytosine content that is between that is between 28.5% and 30% cytosine.

In some embodiments, the coding sequence has an adenine content that is between 35% and 42%, and the portion of the coding sequence that encodes the Plasmodium CSP N-terminal domain has an adenine content that is at least 35%. In some embodiments, the coding sequence has a uracil content that is between 14% and 20%, and the portion of the coding sequence that encodes the Plasmodium CSP N-terminal domain has a uracil content that is at least 10%. In some embodiments, the coding sequence has a guanine content that is between 15% and 19.5%, and the portion of the coding sequence encoding a Plasmodium CSP N-terminal domain has a guanine content that is less than 27%.

In some embodiments, a polyribonucleotide encoding a Plasmodium CSP N-terminal domain comprises a sequence having at least 99%, 98%, 97%, 96%, or 95% identity to SEQ ID NO: 11. In some embodiments, a polyribonucleotide encoding a Plasmodium CSP N-terminal domain comprises or consists of a sequence of SEQ ID NO: 11.

In some embodiments, a polyribonucleotide encoding a Plasmodium CSP N-terminal domain comprises a sequence having at least 99%, 98%, 97%, 96%, or 95% identity to SEQ ID NO: 13. In some embodiments, a polyribonucleotide encoding a Plasmodium CSP N-terminal domain comprises or consists of a sequence of SEQ ID NO: 13.

In some embodiments, a polyribonucleotide encoding a Plasmodium CSP N-terminal domain comprises a sequence having at least 99%, 98%, 97%, 96%, or 95% identity to SEQ ID NO: 15. In some embodiments, a polyribonucleotide encoding a Plasmodium CSP N-terminal domain comprises or consists of a sequence of SEQ ID NO: 15.

In some embodiments, a polyribonucleotide encoding a Plasmodium CSP N-terminal domain comprises a sequence having at least 99%, 98%, 97%, 96%, or 95% identity to SEQ ID NO: 17. In some embodiments, a polyribonucleotide encoding a Plasmodium CSP N-terminal domain comprises or consists of a sequence of SEQ ID NO: 17.

In some embodiments, a polyribonucleotide encoding a Plasmodium CSP secretory signal comprises a sequence having at least 99%, 98%, 97%, 96%, or 95% identity to SEQ ID NO: 6. In some embodiments, a polyribonucleotide encoding a Plasmodium CSP secretory signal comprises or consists of a sequence according to SEQ ID NO: 6

In some embodiments, a polyribonucleotide encoding a Plasmodium CSP minor repeat region comprises a sequence having at least 99%, 98%, 97%, 96%, or 95% identity to SEQ ID NO: 28. In some embodiments, a polyribonucleotide encoding a Plasmodium CSP minor repeat region comprises or consists of a sequence according to SEQ ID NO: 28.

In some embodiments, a polyribonucleotide encoding a Plasmodium CSP major repeat region comprises a sequence having at least 99%, 98%, 97%, 96%, or 95% identity to SEQ ID NO: 32. In some embodiments, a polyribonucleotide encoding a Plasmodium CSP major repeat region comprises or consists of a sequence according to SEQ ID NO: 32.

In some embodiments, a polyribonucleotide encoding a Plasmodium CSP central domain comprises a sequence having at least 99%, 98%, 97%, 96%, or 95% identity to SEQ ID NO: 25. In some embodiments, a polyribonucleotide encoding a Plasmodium CSP central domain comprises or consists of a sequence according to SEQ ID NO: 25.

In some embodiments, a polyribonucleotide encoding a Plasmodium CSP C-terminal region comprises a sequence having at least 99%, 98%, 97%, 96%, or 95% identity to SEQ ID NO: 42. In some embodiments, a polyribonucleotide encoding a Plasmodium CSP C-terminal region comprises or consists of a sequence according to SEQ ID NO: 42.

In some embodiments, a polyribonucleotide encoding a Plasmodium CSP transmembrane region comprises a sequence having at least 99%, 98%, 97%, 96%, or 95% identity to SEQ ID NO: 45. In some embodiments, a polyribonucleotide encoding a Plasmodium CSP transmembrane region comprises or consists of a sequence according to SEQ ID NO: 45.

In some embodiments, a polyribonucleotide encoding a Plasmodium CSP C-terminal domain comprises a sequence having at least 99%, 98%, 97%, 96%, or 95% identity to SEQ ID NO: 39. In some embodiments, a polyribonucleotide encoding a Plasmodium CSP C-terminal domain comprises or consists of a sequence according to SEQ ID NO: 39.

In some embodiments, a polyribonucleotide encoding a Plasmodium CSP full-length polypeptide comprises a sequence having at least 99%, 98%, 97%, 96%, or 95% identity to any one of SEQ ID NOs: 50, 52, 54, 56, 58, 60, and 62. In some embodiments, a polyribonucleotide encoding a Plasmodium CSP full-length polypeptide comprises or consists of a sequence according to any one of SEQ ID NOs: 50, 52, 54, 56, 58, 60, and 62.

In some embodiments, a polyribonucleotide encoding a Plasmodium CSP full-length polypeptide comprises a sequence having at least 99%, 98%, 97%, 96%, or 95% identity to SEQ ID NO: 52. In some embodiments, a polyribonucleotide encoding a Plasmodium CSP full-length polypeptide comprises or consists of a sequence according to SEQ ID NO: 52.

In some embodiments, a polyribonucleotide encoding a Plasmodium CSP full-length polypeptide comprises a sequence having at least 99%, 98%, 97%, 96%, or 95% identity to SEQ ID NO: 54. In some embodiments, a polyribonucleotide encoding a Plasmodium CSP full-length polypeptide comprises or consists of a sequence according to SEQ ID NO: 54.

### I. Exemplary Polyribonucleotide Features

Polyribonucleotides described herein encode a full-length CSP polypeptide construct as described herein. In some embodiments, polyribonucleotides described herein can comprise a nucleotide sequence that encodes a 5′UTR of interest and/or a 3′ UTR of interest. In some embodiments, polynucleotides described herein can comprise a nucleotide sequence that encodes a polyA tail. In some embodiments, polyribonucleotides described herein may comprise a 5′ cap, which may be incorporated during transcription, or joined to a polyribonucleotide post-transcription. A. 5′ Cap

A structural feature of mRNAs is cap structure at five-prime end (5′). Natural eukaryotic mRNA comprises a 7-methylguanosine cap linked to the mRNA via a 5′ to 5′-triphosphate bridge resulting in cap0 structure (m7GpppN). In most eukaryotic mRNA and some viral mRNA, further modifications can occur at the 2′-hydroxy-group (2′-OH) (e.g., the 2′-hydroxyl group may be methylated to form 2′-O-Me) of the first and subsequent nucleotides producing “cap1” and “cap2” five-prime ends, respectively). Diamond, et al., (2014) Cytokine & growth Factor Reviews, 25:543-550, which is incorporated herein by reference in its entirety, reported that cap0-mRNA cannot be translated as efficiently as cap1-mRNA in which the role of 2′-O-Me in the penultimate position at the mRNA 5′ end is determinant. Lack of the 2′-O-met has been shown to trigger innate immunity and activate IFN response. Daffis, et al. (2010) Nature, 468:452-456; and Zust et al. (2011) Nature Immunology, 12:137-143, each of which is incorporated herein by reference in its entirety.

RNA capping is well researched and is described, e.g., in Decroly E et al. (2012) Nature Reviews 10: 51-65; and in Ramanathan A. et al., (2016) Nucleic Acids Res; 44(16): 7511-7526, the entire contents of each of which is hereby incorporated by reference. For example, in some embodiments, a 5′-cap structure which may be suitable in the context of the present invention is a cap0 (methylation of the first nucleobase, e.g., m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (“anti-reverse cap analogue”), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

The term “5′-cap” as used herein refers to a structure found on the 5′-end of an RNA, e.g., mRNA, and generally includes a guanosine nucleotide connected to an RNA, e.g., mRNA, via a 5′- to 5′-triphosphate linkage (also referred to as Gppp or G(5′)ppp(5′)). In some embodiments, a guanosine nucleoside included in a 5′ cap may be modified, for example, by methylation at one or more positions (e.g., at the 7-position) on a base (guanine), and/or by methylation at one or more positions of a ribose. In some embodiments, a guanosine nucleoside included in a 5′ cap comprises a 3′O methylation at a ribose (3′OMeG). In some embodiments, a guanosine nucleoside included in a 5′ cap comprises methylation at the 7-position of guanine (m7G). In some embodiments, a guanosine nucleoside included in a 5′ cap comprises methylation at the 7-position of guanine and a 3′ O methylation at a ribose (m7(3′OMeG)). It will be understood that the notation used in the above paragraph, e.g., “(m₂^{7,3′-O})G” or “m7(3′OMeG)”, applies to other structures described herein.

In some embodiments, providing an RNA with a 5′-cap disclosed herein may be achieved by in vitro transcription, in which a 5′-cap is co-transcriptionally expressed into an RNA strand, or may be attached to an RNA post-transcriptionally using capping enzymes. In some embodiments, co-transcriptional capping with a cap disclosed improves the capping efficiency of an RNA compared to co-transcriptional capping with an appropriate reference comparator. In some embodiments, improving capping efficiency can increase a translation efficiency and/or translation rate of an RNA, and/or increase expression of an encoded polypeptide. In some embodiments, alterations to polynucleotides generates a non-hydrolyzable cap structure which can, for example, prevent decapping and increase RNA half-life.

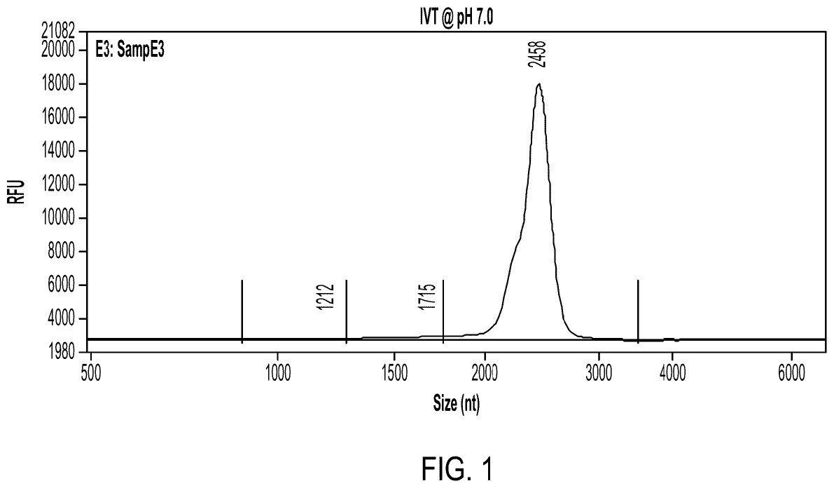

In some embodiments, a utilized 5′ caps is a cap0, a cap1, or cap2 structure. See, e.g., FIG. 1 of Ramanathan A et al., and FIG. 1 of Decroly E et al., each of which is incorporated herein by reference in its entirety. See, e.g., FIG. 1 of Ramanathan A et al., and FIG. 1 of Decroly E et al., each of which is incorporated herein by reference in its entirety. In some embodiments, an RNA described herein comprises a cap1 structure. In some embodiments, an RNA described herein comprises a cap2.

In some embodiments, an RNA described herein comprises a cap0 structure. In some embodiments, a cap0 structure comprises a guanosine nucleoside methylated at the 7-position of guanine ((m⁷)G). In some embodiments, such a cap0 structure is connected to an RNA via a 5′- to 5′-triphosphate linkage and is also referred to herein as (m⁷)Gppp. In some embodiments, a cap0 structure comprises a guanosine nucleoside methylated at the 2′-position of the ribose of guanosine. In some embodiments, a cap0 structure comprises a guanosine nucleoside methylated at the 3′-position of the ribose of guanosine. In some embodiments, a guanosine nucleoside included in a 5′ cap comprises methylation at the 7-position of guanine and at the 2′-position of the ribose ((m₂^{7,2′-O})G). In some embodiments, a guanosine nucleoside included in a 5′ cap comprises methylation at the 7-position of guanine and at the 2′-position of the ribose ((m₂^{7,3′-O})G).

In some embodiments, a cap1 structure comprises a guanosine nucleoside methylated at the 7-position of guanine ((m⁷)G) and optionally methylated at the 2′ or 3′ position pf the ribose, and a 2′O methylated first nucleotide in an RNA ((m^{2′-O})N₁). In some embodiments, a cap1 structure comprises a guanosine nucleoside methylated at the 7-position of guanine ((m⁷)G) and the 3′ position of the ribose, and a 2′O methylated first nucleotide in an RNA ((m^{2′-O})N₁). In some embodiments, a cap1 structure is connected to an RNA via a 5′- to 5′-triphosphate linkage and is also referred to herein as, e.g., ((m⁷)Gppp(^{2′-O})N₁) or (m₂^{7,3′-O})Gppp(^{2′-O})N₁), where N₁ is as defined and described herein. In some embodiments, a cap1 structure comprises a second nucleotide, N₂, which is at position 2 and is chosen from A, G, C, or U, e.g., (m⁷)Gppp(^{2′-O})N₁pN₂ or (m₂^{7,3′-O})Gppp(^{2′-O})N₁pN₂, where each of N₁ and N₂ is as defined and described herein.

In some embodiments, a cap2 structure comprises a guanosine nucleoside methylated at the 7-position of guanine ((m⁷)G) and optionally methylated at the 2′ or 3′ position of the ribose, and a 2′O methylated first and second nucleotides in an RNA ((m^{2′-O})N₁p(m^{2′-O})N₂). In some embodiments, a cap2 structure comprises a guanosine nucleoside methylated at the 7-position of guanine ((m⁷)G) and the 3′ position of the ribose, and a 2′O methylated first and second nucleotide in an RNA. In some embodiments, a cap2 structure is connected to an RNA via a 5′- to 5′-triphosphate linkage and is also referred to herein as, e.g., ((m⁷)Gppp(^{2′-O})N₁p(^{2′-O})N₂) or (m₂^{7,3′-O})Gppp(^{2′-O})N₁p(^{2′-O})N₂), where each of N₁ and N₂ is as defined and described herein.

In some embodiments, the 5′ cap is a dinucleotide cap structure. In some embodiments, the 5′ cap is a dinucleotide cap structure comprising N₁, where N₁ is as defined and described herein. In some embodiments, the 5′ cap is a dinucleotide cap G*N₁, where N₁ is as defined above and herein, and G* comprises a structure of formula (I):

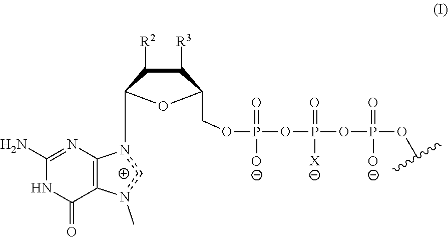

or a salt thereof,
where each R² and R³ is —OH or —OCH₃; and X is O or S.

In some embodiments, R² is —OH. In some embodiments, R² is —OCH₃. In some embodiments, R³ is —OH. In some embodiments, R³ is —OCH₃. In some embodiments, R² is —OH and R³ is —OH. In some embodiments, R² is —OH and R³ is —CH₃. In some embodiments, R² is —CH₃ and R³ is —OH. In some embodiments, R² is —CH₃ and R³ is —CH₃.

In some embodiments, X is O. In some embodiments, X is S.

In some embodiments, the 5′ cap is a dinucleotide cap0 structure (e.g., (m⁷)GpppN₁, (m₂^{7,2′-O}) GpppN₁, (m₂^{7,3′-O})GpppN₁, (m⁷)GppSpN₁, (m₂^{7,2′-O})GppSpN₁, or (m₂^{7,3′-O})GppSpN₁), where N₁ is as defined and described herein. In some embodiments, the 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O})GppSpN₁, or (m₂^{7,3′-O})GppSpN₁), where N₁ is G. In some embodiments, the 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O}) GppSpN₁, or (m₂^{7,3′-O})GppSpN₁), where N₁ is A, U, or C. In some embodiments, the 5′ cap is a dinucleotide cap1 structure (e.g., (m⁷)Gppp(m^{2′-O})N₁, (m₂^{7,2′-O})Gppp(m^{2′-O})N₁, (m₂₇₃′-⁰)Gppp(m^{2′-O})N₁, (m⁷)GppSp(m^{2′-O})N₁, (m₂^{7,2′-O}) GppSp(m^{2′-O})N₁, or (m₂^{7,3′-O})GppSp(m^{2′-O})N₁), where N₁ is as defined and described herein. In some embodiments, the 5′ cap is selected from the group consisting of (m⁷)GpppG (“Ecap0”), (m⁷)Gppp(m^{2′-O})G (“Ecap1”), (m₂^{7,3′-O})GpppG (“ARCA” or “D1”), and (m₂^{7,2′-O})GppSpG (“beta-S-ARCA”). In some embodiments, the 5′ cap is (m⁷)GpppG (“Ecap0”), having a structure:

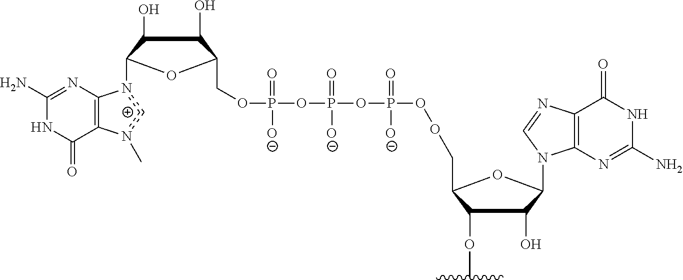

or a salt thereof.

In some embodiments, the 5′ cap is (m⁷)Gppp(m^{2′-O})G (“Ecap1”), having a structure:

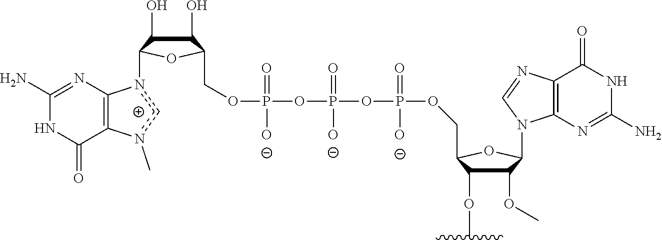

or a salt thereof.

In some embodiments, the 5′ cap is (m₂^{7,3′-O})GpppG (“ARCA” or “D1”), having a structure:

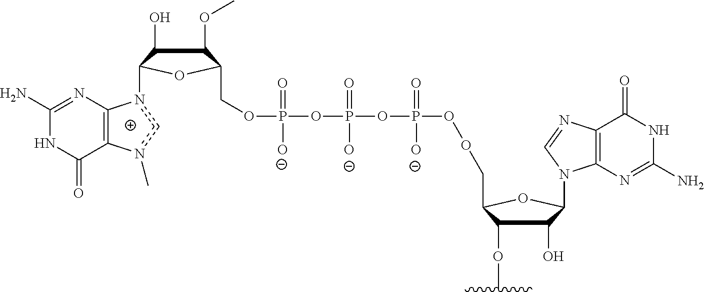

or a salt thereof.

In some embodiments, the 5′ cap is (m₂^{7,2′-O})GppSpG (“beta-S-ARCA”), having a structure:

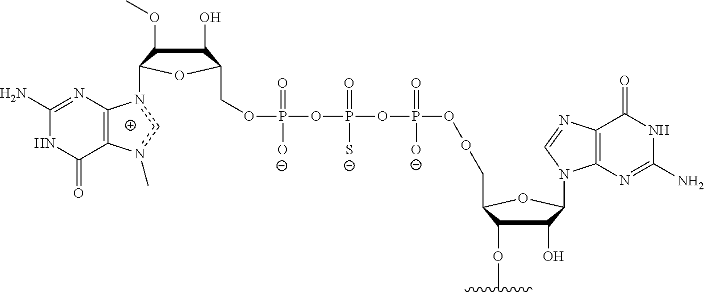

or a salt thereof.

In some embodiments, the 5′ cap is a trinucleotide cap structure. In some embodiments, the 5′ cap is a trinucleotide cap structure comprising N₁pN₂, where N₁ and N₂ are as defined and described herein. In some embodiments, the 5′ cap is a dinucleotide cap G*N₁pN₂, where N₁ and N₂ are as defined above and herein, and G* comprises a structure of formula (I):

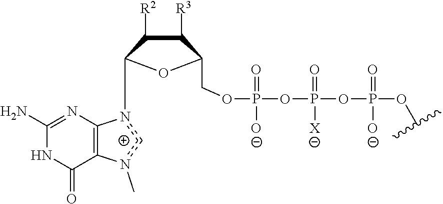

or a salt thereof, where R², R³, and X are as defined and described herein.

In some embodiments, the 5′ cap is a trinucleotide cap0 structure (e.g. (m⁷)GpppN₁pN₂, (m₂^{7,2′-O}) GpppN₁pN₂, or (m₂^{7,3′-O})GpppN₁pN₂), where N₁ and N₂ are as defined and described herein). In some embodiments, the 5′ cap is a trinucleotide cap1 structure (e.g., (m⁷)Gppp(m^{2′-O})N₁pN₂, (m₂^{7,2′-O})Gppp(m^{2′-O})N₁pN₂, (m₂^{7,3′-O})Gppp(m^{2′-O}) N₁pN₂), where N₁ and N₂ are as defined and described herein. In some embodiments, the 5′ cap is a trinucleotide cap2 structure (e.g., (m⁷)Gppp(m^{2′-O})N₁p(m^{2′-O})N₂, (m₂^{7,2′-O})Gppp(m^{2′-O})N₁p(m^{2′-O})N₂, (m₂^{7,3′-O})Gppp(m^{2′-O})N₁p(m^{2′-O}) N₂), where N₁ and N₂ are as defined and described herein. In some embodiments, the 5′ cap is selected from the group consisting of (m₂^{7,3′-O})Gppp(m^{2′-O})ApG (“CleanCap AG”, “CC413”), (m₂^{7,3′-O})Gppp(m^{2′-O})GpG (“CleanCap GG”), (m⁷)Gppp(m^{2′-O})ApG, (m⁷)Gppp(m^{2′-O})GpG, (m₂^{7,3′-O})Gppp(m₂^{6,2′-O})ApG, and (m⁷)Gppp(m^{2′-O})ApU.

In some embodiments, the 5′ cap is (m₂^{7,3′-O})Gppp(m^{2′-O})ApG (“CleanCap AG”, “CC413”), having a structure:

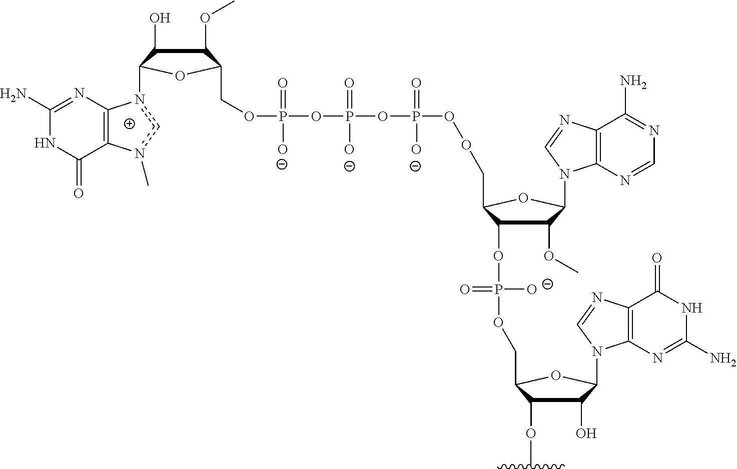

or a salt thereof.

In some embodiments, the 5′ cap is (m₂^{7,3′-O})Gppp(m^{2′-O})GpG (“CleanCap GG”), having a structure:

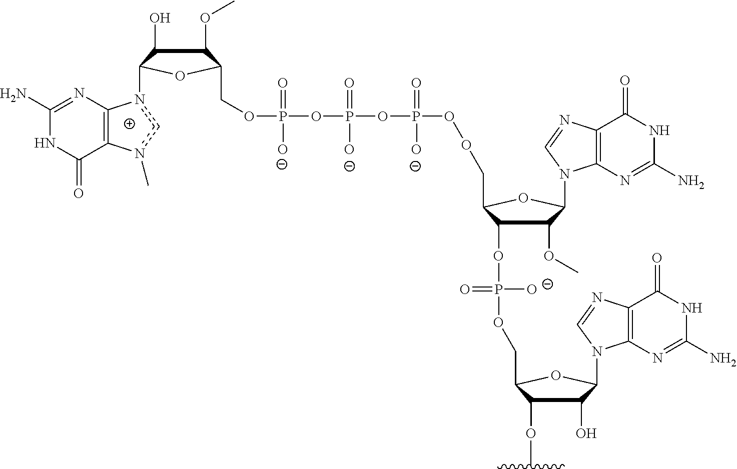

or a salt thereof.

In some embodiments, the 5′ cap is (m⁷)Gppp(m^{2′-O})ApG, having a structure:

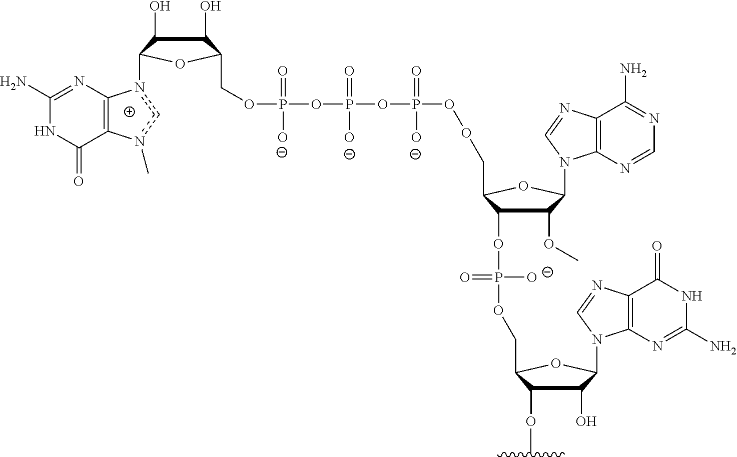

or a salt thereof.

In some embodiments, the 5′ cap is (m⁷)Gppp(m^{2′-O})GpG, having a structure:

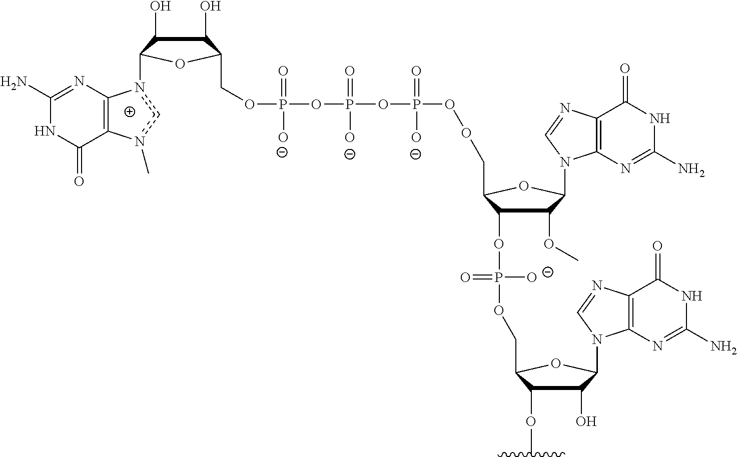

or a salt thereof.

In some embodiments, the 5′ cap is (m₂^{7,3′-O})Gppp(m₂^{6,2′-O})ApG, having a structure:

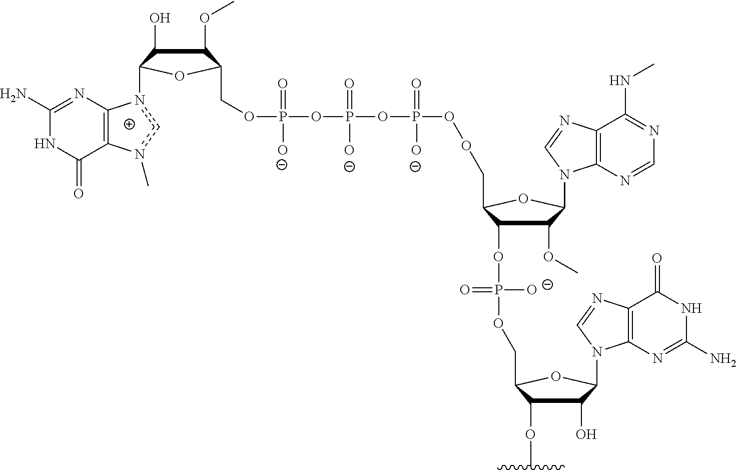

or a salt thereof.

In some embodiments, the 5′ cap is (m⁷)Gppp(m^{2′-O})ApU, having a structure:

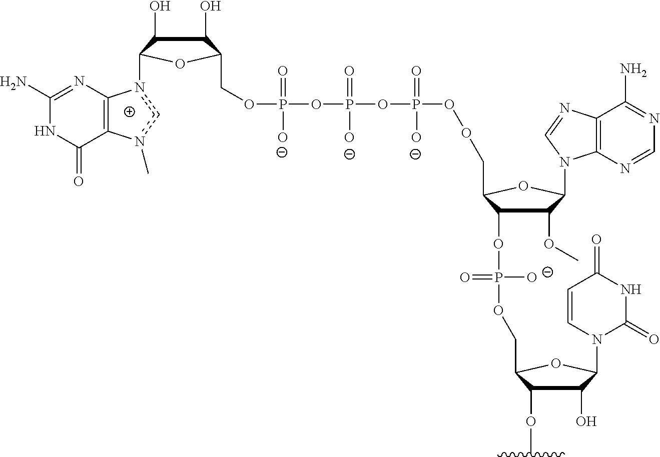

or a salt thereof.

In some embodiments, the 5′ cap is a tetranucleotide cap structure. In some embodiments, the 5′ cap is a tetranucleotide cap structure comprising N₁pN₂pN₃, where N₁, N₂, and N₃ are as defined and described herein. In some embodiments, the 5′ cap is a tetranucleotide cap G*N₁pN₂pN₃, where N₁, N₂, and N₃ are as defined above and herein, and G* comprises a structure of formula (I):

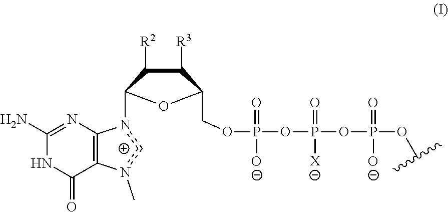

or a salt thereof, where R², R³, and X are as defined and described herein.

In some embodiments, the 5′ cap is a tetranucleotide cap0 structure (e.g. (m⁷)GpppN₁pN₂pN₃, (m₂^{7,2′-O}) GpppN₁pN₂pN₃, or (m₂^{7,3′-O})GpppN₁N₂pN₃), where N₁, N₂, and N₃ are as defined and described herein). In some embodiments, the 5′ cap is a tetranucleotide Cap1 structure (e.g., (m⁷)Gppp(m^{2′-O})N₁pN₂pN₃, (m₂^{7,2′-O})Gppp(m^{2′-O}) N₁pN₂pN₃, (m₂^{7,3′-O})Gppp(m^{2′-O})N₁pN₂N₃), where N₁, N₂, and N₃ are as defined and described herein. In some embodiments, the 5′ cap is a tetranucleotide Cap2 structure (e.g., (m⁷)Gppp(m^{2′-O})N₁p(m^{2′-O})N₂pN₃, (m₂^{7,2′-O})Gppp(m^{2′-O}) N₁p(m^{2′-O})N₂pN₃, (m₂^{7,3′-O})Gppp(m^{2′-O})N₁p(m^{2′-O})N₂pN₃), where N₁, N₂, and N₃ are as defined and described herein. In some embodiments, the 5′ cap is selected from the group consisting of (m₂^{7,3′-O})Gppp(m^{2′-O})Ap(m^{2′-O})GpG, (m₂^{7,3′-O}) Gppp(m^{2′-O})Gp(m^{2′-O})GpC, (m⁷)Gppp(m^{2′-O})Ap(m^{2′-O})UpA, and (m⁷)Gppp(m^{2′-O})Ap(m^{2′-O})GpG.

In some embodiments, the 5′ cap is (m₂^{7,3′-O})Gppp(m^{2′-O})Ap(m^{2′-O})GpG, having a structure:

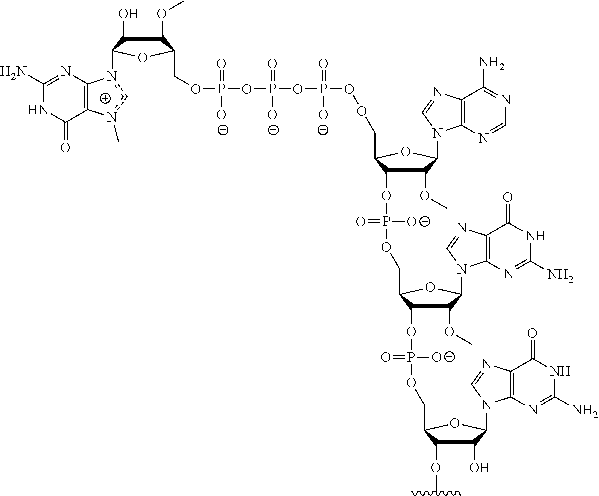

or a salt thereof.

In some embodiments, the 5′ cap is (m₂^{7,3′-O})Gppp(m^{2′-O})Gp(m^{2′-O})GpC, having a structure:

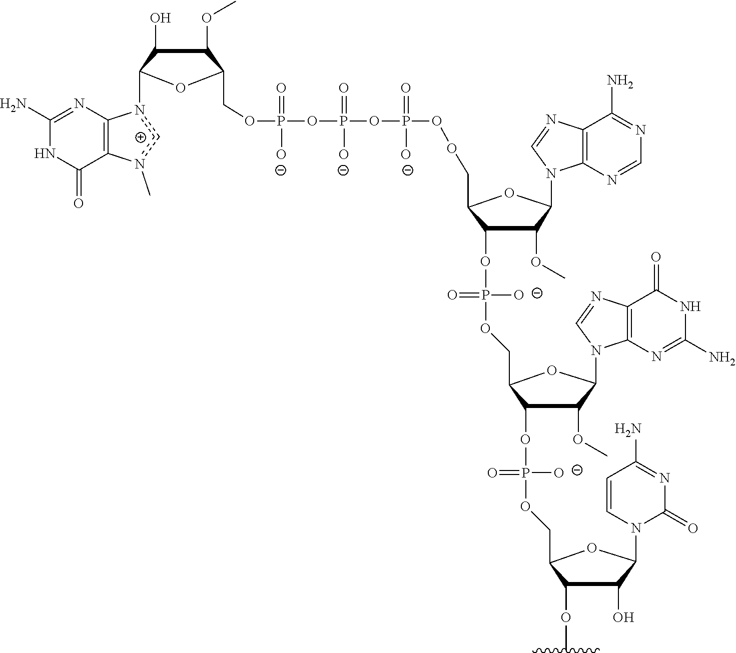

or a salt thereof.

In some embodiments, the 5′ cap is (m⁷)Gppp(m^{2′-O})Ap(m^{2′-O})UpA, having a structure:

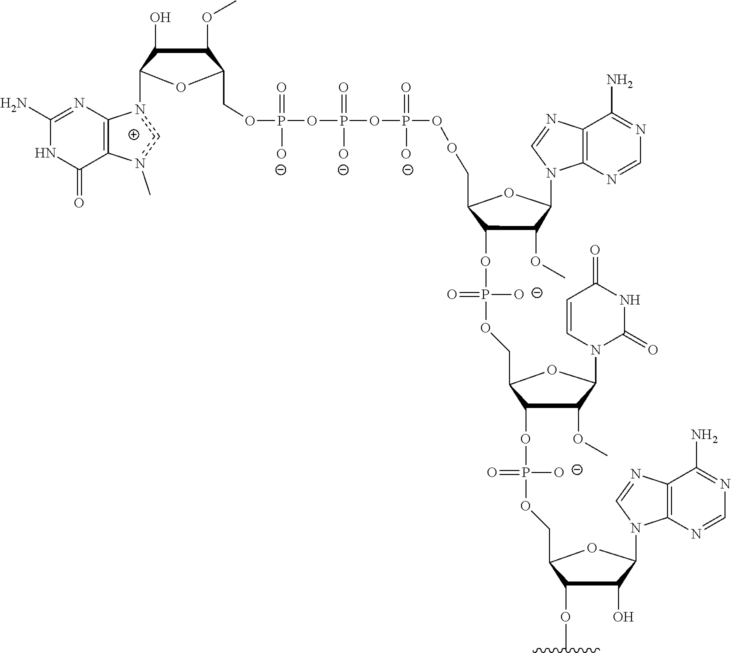

or a salt thereof.

In some embodiments, the 5′ cap is (m⁷)Gppp(m^{2′-O})Ap(m^{2′-O})GpG, having a structure:

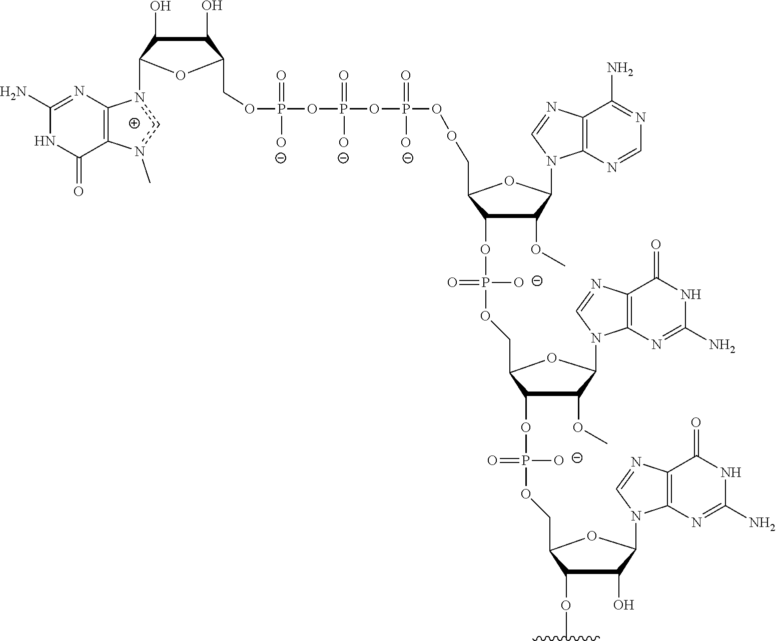

or a salt thereof.

### B. Cap Proximal Sequences

In some embodiments, a 5′ UTR utilized in accordance with the present disclosure comprises a cap proximal sequence, e.g., as disclosed herein. In some embodiments, a cap proximal sequence comprises a sequence adjacent to a 5′ cap. In some embodiments, a cap proximal sequence comprises nucleotides in positions +1, +2, +3, +4, and/or +5 of an RNA polynucleotide.

In some embodiments, a cap structure comprises one or more polynucleotides of a cap proximal sequence. In some embodiments, a cap structure comprises an m⁷ Guanosine cap and nucleotide+1 (N₁) of an RNA polynucleotide. In some embodiments, a cap structure comprises an m⁷ Guanosine cap and nucleotide+2 (N₂) of an RNA polynucleotide. In some embodiments, a cap structure comprises an m⁷ Guanosine cap and nucleotides+1 and +2 (N₁ and N₂) of an RNA polynucleotide. In some embodiments, a cap structure comprises an m⁷ Guanosine cap and nucleotides+1, +2, and +3 (N₁, N₂, and N₃) of an RNA polynucleotide.

Those skilled in the art, reading the present disclosure, will appreciate that, in some embodiments, one or more residues of a cap proximal sequence (e.g., one or more of residues +1, +2, +3, +4, and/or +5) may be included in an RNA by virtue of having been included in a cap entity (e.g., a cap1 or cap2 structure, etc.); alternatively, in some embodiments, at least some of the residues in a cap proximal sequence may be enzymatically added (e.g., by a polymerase such as a T7 polymerase). For example, in certain exemplified embodiments where a m₂^{7,3′-O}Gppp(m₁^{2′-O}) ApG cap is utilized, +1 (i.e., N₁) and +2 (i.e. N₂) are the (m₁^{2′-O})A and G residues of the cap, and +3, +4, and +5 are added by polymerase (e.g., T7 polymerase).

In some embodiments, the 5′ cap is a dinucleotide cap structure, where the cap proximal sequence comprises N₁ of the 5′ cap, where N₁ is any nucleotide, e.g., A, C, G or U. In some embodiments, the 5′ cap is a trinucleotide cap structure (e.g., the trinucleotide cap structures described above and herein), where the cap proximal sequence comprises N₁ and N₂ of the 5′ cap, where N₁ and N₂ are independently any nucleotide, e.g., A, C, G or U. In some embodiments, the 5′ cap is a tetranucleotide cap structure (e.g., the trinucleotide cap structures described above and herein), where the cap proximal sequence comprises N₁, N₂, and N₃ of the 5′ cap, where N₁, N₂, and N₃ are any nucleotide, e.g., A, C, G or U.

In some embodiments, e.g., where the 5′ cap is a dinucleotide cap structure, a cap proximal sequence comprises N₁ of a the 5′ cap, and N₂, N₃, N₄ and N₅, where N₁ to N₅ correspond to positions +1, +2, +3, +4, and/or +5 of an RNA polynucleotide. In some embodiments, e.g., where the 5′ cap is a trinucleotide cap structure, a cap proximal sequence comprises N₁ and N₂ of a the 5′ cap, and N₃, N₄ and N₅, where N₁ to N₅ correspond to positions +1, +2, +3, +4, and/or +5 of an RNA polynucleotide. In some embodiments, e.g., where the 5′ cap is a tetranucleotide cap structure, a cap proximal sequence comprises N₁, N₂, and N₃ of a the 5′ cap, and N₄ and N₅, where N₁ to N₅ correspond to positions +1, +2, +3, +4, and/or +5 of an RNA polynucleotide.

In some embodiments, N₁ is A. In some embodiments, N₁ is C. In some embodiments, N₁ is G. In some embodiments, N₁ is U. In some embodiments, N₂ is A. In some embodiments, N₂ is C. In some embodiments, N₂ is G. In some embodiments, N₂ is U. In some embodiments, N₃ is A. In some embodiments, N₃ is C. In some embodiments, N₃ is G. In some embodiments, N₃ is U. In some embodiments, N₄ is A. In some embodiments, N₄ is C. In some embodiments, N₄ is G. In some embodiments, N₄ is U. In some embodiments, N₅ is A. In some embodiments, N₅ is C. In some embodiments, N₅ is G. In some embodiments, N₅ is U. It will be understood that, each of the embodiments described above and herein (e.g., for N₁ through N₅) may be taken singly or in combination and/or may be combined with other embodiments of variables described above and herein (e.g., 5′ caps).

### C. 5′ UTR

In some embodiments, a nucleic acid (e.g., DNA, RNA) utilized in accordance with the present disclosure comprises a 5′-UTR. In some embodiments, 5′-UTR may comprise a plurality of distinct sequence elements; in some embodiments, such plurality may be or comprise multiple copies of one or more particular sequence elements (e.g., as may be from a particular source or otherwise known as a functional or characteristic sequence element). In some embodiments a 5′ UTR comprises multiple different sequence elements.

The term “untranslated region” or “UTR” is commonly used in the art to a region in a DNA molecule which is transcribed but is not translated into an amino acid sequence, or to the corresponding region in an RNA polynucleotide, such as an mRNA molecule. An untranslated region (UTR) can be present 5′ (upstream) of an open reading frame (5′-UTR) and/or 3′ (downstream) of an open reading frame (3′-UTR). As used herein, the terms “five prime untranslated region” or “5′ UTR” refer to a sequence of a polyribonucleotide between the 5′ end of the polyribonucleotide (e.g., a transcription start site) and a start codon of a coding region of the polyribonucleotide. In some embodiments, “5′ UTR” refers to a sequence of a polyribonucleotide that begins at the 5′ end of the polyribonucleotide (e.g., a transcription start site) and ends one nucleotide (nt) before a start codon (usually AUG) of a coding region of the polyribonucleotide, e.g., in its natural context. In some embodiments, a 5′ UTR comprises a Kozak sequence. A 5′-UTR is downstream of the 5′-cap (if present), e.g., directly adjacent to the 5′-cap. In some embodiments, a 5′ UTR disclosed herein comprises a cap proximal sequence, e.g., as defined and described herein. In some embodiments, a cap proximal sequence comprises a sequence adjacent to a 5′ cap.

Exemplary 5′ UTRs include a human alpha globin (hAg) 5′UTR or a fragment thereof, a TEV 5′ UTR or a fragment thereof, a HSP70 5′ UTR or a fragment thereof, or a c-Jun 5′ UTR or a fragment thereof.

In some embodiments, an RNA disclosed herein comprises a hAg 5′ UTR or a fragment thereof.

In some embodiments, an RNA disclosed herein comprises a 5′ UTR having at least 80%, at least 85%, at least 90%, at least 95%, at least 96%, at least 97%, at least 98%, or at least 99% identity to a 5′ UTR with the sequence AGAAUAAACUAGUAUUCUUCUGGUCCCCACAGACUCAGAGAGAACCCGCCACC (SEQ ID NO: 63). In some embodiments, an RNA disclosed herein comprises a 5′ UTR having the sequence AGAAUAAACUAGUAUUCUUCUGGUCCCCACAGACUCAGAGAGAACCCGCCACC (SEQ ID NO: 63).

### D. PolyA Tail

In some embodiments, a polynucleotide (e.g., DNA, RNA) disclosed herein comprises a polyadenylate (polyA) sequence, e.g., as described herein. In some embodiments, a polyA sequence is situated downstream of a 3′-UTR, e.g., adjacent to a 3′-UTR.

As used herein, the term “poly(A) sequence” or “poly-A tail” refers to an uninterrupted or interrupted sequence of adenylate residues which is typically located at the 3′-end of an RNA polynucleotide. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. In some embodiments, polynucleotides disclosed herein comprise an uninterrupted Poly(A) sequence. In some embodiments, polynucleotides disclosed herein comprise interrupted Poly(A) sequence. In some embodiments, RNAs disclosed herein can have a poly(A) sequence attached to the free 3-end of the RNA by a template-independent RNA polymerase after transcription or a poly(A) sequence encoded by DNA and transcribed by a template-dependent RNA polymerase.

It has been demonstrated that a poly(A) sequence of about 120 A nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017, which is herein incorporated by reference).

In some embodiments, a poly(A) sequence in accordance with the present disclosure is not limited to a particular length; in some embodiments, a poly(A) sequence is any length. In some embodiments, a poly(A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1, which is incorporated herein by reference in its entirety, may be used in accordance with the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. In some embodiments, the poly(A) sequence contained in an RNA polynucleotide described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly(A) sequence at its 3′-end, i.e., the poly(A) sequence is not masked or followed at its 3-end by a nucleotide other than A.

In some embodiments, the poly(A) sequence may comprise at least 20, at least 30, at least 40, at least 80, or at least 100 and up to 500, up to 400, up to 300, up to 200, or up to 150 nucleotides. In some embodiments, the poly(A) sequence may essentially consist of at least 20, at least 30, at least 40, at least 80, or at least 100 and up to 500, up to 400, up to 300, up to 200, or up to 150 nucleotides. In some embodiments, the poly(A) sequence may consist of at least 20, at least 30, at least 40, at least 80, or at least 100 and up to 500, up to 400, up to 300, up to 200, or up to 150 nucleotides. In some embodiments, the poly(A) sequence comprises at least 100 nucleotides. In some embodiments, the poly(A) sequence comprises about 150 nucleotides. In some embodiments, the poly(A) sequence comprises about 120 nucleotides.

In some embodiments, a poly A tail comprises a specific number of Adenosines, such as about 50 or more, about 60 or more, about 70 or more, about 80 or more, about 90 or more, about 100 or more, about 120, or about 150 or about 200. In some embodiments a poly A tail of a string construct may comprise 200 A residues or less. In some embodiments, a poly A tail of a string construct may comprise about 200 A residues. In some embodiments, a poly A tail of a string construct may comprise 180 A residues or less. In some embodiments, a poly A tail of a string construct may comprise about 180 A residues. In some embodiments, a poly A tail may comprise 150 residues or less.

In some embodiments, RNA comprises a poly(A) sequence comprising the nucleotide sequence of AAAAAAAAAAAAAAAAAAAAAAAAAAAAAAGCAUAUGACUAAAAAAAAAAAAAAAAAAAAAAAAAAAAAAAAAAAAAAAAAAAAAA AAAAAAAAAAAAAAAAAAAAAAAA (SEQ ID NO: 66), or a nucleotide sequence having at least 99%, 98%, 97%, 96%, 95%, 90%, 85%, or 80% identity to the nucleotide sequence of AAAAAAAAAAAAAAAAAAAAAAAAAAAAAAGCAUAUGACUAAAAAAAAAAAAAAAAAAAAAAAAAAAAAAAAAAAAAAAAAAAAAA AAAAAAAAAAAAAAAAAAAAAAAA (SEQ ID NO: 66). In some embodiments, a poly(A) tail comprises a plurality of A residues interrupted by a linker. In some embodiments, a linker comprises the nucleotide sequence GCAUAUGAC. In some embodiments, a linker comprises the nucleotide sequence GCAUAUGACU (SEQ ID NO: 68).

### E. 3′ UTR

In some embodiments, an RNA utilized in accordance with the present disclosure comprises a 3′-UTR. As used herein, the terms “three prime untranslated region,” “3′ untranslated region,” or “3′ UTR” refer to a sequence of an mRNA molecule that begins following a stop codon of a coding region of an open reading frame sequence. In some embodiments, the 3′ UTR begins immediately after a stop codon of a coding region of an open reading frame sequence, e.g., in its natural context. In other embodiments, the 3′ UTR does not begin immediately after stop codon of the coding region of an open reading frame sequence, e.g., in its natural context. The term “3′-UTR” does preferably not include the poly(A) sequence. Thus, the 3′-UTR is upstream of the poly(A) sequence (if present), e.g. directly adjacent to the poly(A) sequence.

In some embodiments, an RNA disclosed herein comprises a 3′ UTR comprising an F element and/or an I element. In some embodiments, a 3′ UTR or a proximal sequence thereto comprises a restriction site. In some embodiments, a restriction site is a BamHIsite. In some embodiments, a restriction site is a XhoIsite.

In some embodiments, an RNA construct comprises an F element. In some embodiments, a F element sequence is a 3′-UTR of amino-terminal enhancer of split (AES).

In some embodiments, an RNA disclosed herein comprises a 3′ UTR having at least 80%, at least 85%, at least 90%, at least 95%, at least 96%, at least 97%, at least 98%, or at least 99% identity to a 3′ UTR with the sequence of CUGGUACUGCAUGCACGCAAUGCUAGCUGCCCCUUUCCCGUCCUGGGUACCCCGAGUCUCCCCCGACCUCGGGUCCCAGGUA UGCUCCCACCUCCACCUGCCCCACUCACCACCUCUGCUAGUUCCAGACACCUCCCAAGCACGCAGCAAUGCAGCUCAAAACGCU UAGCCUAGCCACACCCCCACGGGAAACAGCAGUGAUUAACCUUUAGCAAUAAACGAAAGUUUAACUAAGCUAUACUAACCCCA GGGUUGGUCAAUUUCGUGCCAGCCACACC (SEQ ID NO: 64). In some embodiments, an RNA disclosed herein comprises a 3′ UTR with the sequence of CUGGUACUGCAUGCACGCAAUGCUAGCUGCCCCUUUCCCGUCCUGGGUACCCCGAGUCUCCCCCGACCUCGGGUCCCAGGUA UGCUCCCACCUCCACCUGCCCCACUCACCACCUCUGCUAGUUCCAGACACCUCCCAAGCACGCAGCAAUGCAGCUCAAAACGCU UAGCCUAGCCACACCCCCACGGGAAACAGCAGUGAUUAACCUUUAGCAAUAAACGAAAGUUUAACUAAGCUAUACUAACCCCA GGGUUGGUCAAUUUCGUGCCAGCCACACC (SEQ ID NO: 64).

In some embodiments, a 3′UTR is an FI element as described in WO2017/060314, which is herein incorporated by reference in its entirety.

### III. Exemplary RNA Constructs

The present disclosure provides RNA constructs encoding a full-length Plasmodium CSP polypeptide having beneficial characteristics, e.g., for manufacturing. The present disclosure recognizes a problem with certain RNA constructs encoding a full-length Plasmodium CSP polypeptide, in that their integrity cannot be confirmed using convention methods (e.g., capillary electrophoresis, e.g., a fragment analyzer). For example, in some embodiments an exemplary construct, RNA construct 23, having an RNA sequence of SEQ ID NO: 69, is associated with a double peak on a fragment analyzer. The present disclosure provides RNA constructs that, among other things, overcome this problem.

In some embodiments, an RNA construct encoding a Plasmodium CSP full-length polypeptide comprises a sequence having at least 99%, 98%, 97%, 96%, or 95% identity to any one of SEQ ID NOs: 70 to 76. In some embodiments, an RNA construct encoding a Plasmodium CSP full-length polypeptide comprises or consists of a sequence according to any one of SEQ ID NOs: 70 to 76.

In some embodiments, an RNA construct encoding a Plasmodium CSP full-length polypeptide comprises a sequence having at least 99%, 98%, 97%, 96%, or 95% identity to SEQ ID NO: 71. In some embodiments, an RNA construct encoding a Plasmodium CSP full-length polypeptide comprises or consists of a sequence according to SEQ ID NO: 71.

In some embodiments, an RNA construct encoding a Plasmodium CSP full-length polypeptide comprises a sequence having at least 99%, 98%, 97%, 96%, or 95% identity to SEQ ID NO: 72. In some embodiments, an RNA construct encoding a Plasmodium CSP full-length polypeptide comprises or consists of a sequence according to SEQ ID NO: 72.

### IV. RNA Formats

At least three distinct formats useful for RNA compositions (e.g., pharmaceutical compositions) have been developed, namely non-modified uridine containing mRNA (uRNA), nucleoside-modified mRNA (modRNA), and self-amplifying mRNA (saRNA). Each of these platforms displays unique features. In general, in all three formats, RNA is capped, contains open reading frames (ORFs) flanked by untranslated regions (UTR), and have a polyA-tail at the 3′ end. An ORF of an uRNA and modRNA vectors encode an antibody agent or portion thereof. An saRNA has multiple ORFs.

In some embodiments, the RNA described herein may have modified nucleosides. In some embodiments, the RNA comprises a modified nucleoside in place of at least one (e.g., every) uridine.

The term “uracil,” as used herein, describes one of the nucleobases that can occur in the nucleic acid of RNA. The structure of uracil is:

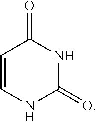

The term “uridine,” as used herein, describes one of the nucleosides that can occur in RNA. The structure of uridine is:

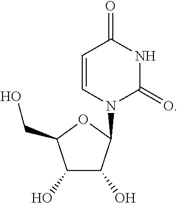 [SMILES: O=c1ccn([C@@H]2O[C@H](CO)[C@@H](O)[C@H]2O)c(=O)[nH]1]

UTP (uridine 5′-triphosphate) has the following structure:

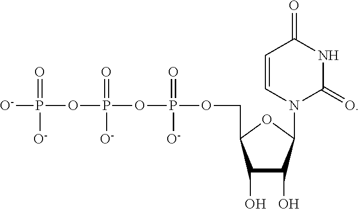

Pseudo-UTP (pseudouridine 5′-triphosphate) has the following structure:

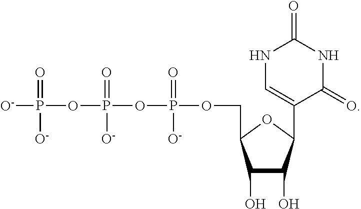

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1L4J), which has the structure:

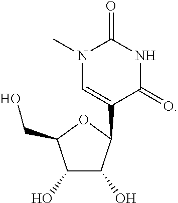 [SMILES: Cn1cc([C@@H]2O[C@H](CO)[C@@H](O)[C@H]2O)c(=O)[nH]c1=O]

N1-methyl-pseudo-UTP has the following structure:

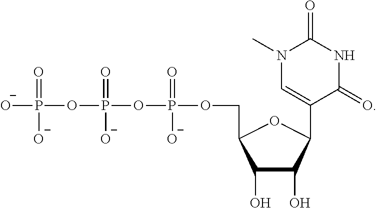

Another exemplary modified nucleoside is 5-methyl-uridine (m5U), which has the structure:

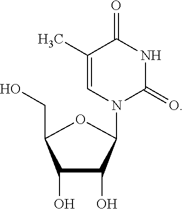

In some embodiments, one or more uridine in the RNA described herein is replaced by a modified nucleoside. In some embodiments, the modified nucleoside is a modified uridine.

In some embodiments, RNA comprises a modified nucleoside in place of at least one uridine. In some embodiments, RNA comprises a modified nucleoside in place of each uridine.

In some embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, the modified nucleoside replacing one or more, e.g., all, uridine in the RNA may be any one or more of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (Tm5U), 1-taurinomethyl-pseudouridine, 5-taurinomethyl-2-thio-uridine(Tm5s2U), 1-taurinomethyl-4-thio-pseudouridine), 5-methyl-2-thio-uridine (m5s2U), 1-methyl-4-thio-pseudouridine (m1s4ψ), 4-thio-1-methyl-pseudouridine, 3-methyl-pseudouridine (m3ty), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 (p), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′—OH-ara-uridine, 5-(2-carbomethoxyvinyl) uridine, 5-[3-(1-E-propenylamino)uridine, or any other modified uridine known in the art.

In some embodiments, the RNA comprises other modified nucleosides or comprises further modified nucleosides, e.g., modified cytidine. For example, in some embodiments, in the RNA 5-methylcytidine is substituted partially or completely, preferably completely, for cytidine. In some embodiments, the RNA comprises 5-methylcytidine and one or more selected from pseudouridine (ψ), N1-methyl-pseudouridine (m1ψ), and 5-methyl-uridine (m5U). In some embodiments, the RNA comprises 5-methylcytidine and N1-methyl-pseudouridine (m1ψ). In some embodiments, the RNA comprises 5-methylcytidine in place of each cytidine and N1-methyl-pseudouridine (m1ψ) in place of each uridine.

In some embodiments of the present disclosure, the RNA is “replicon RNA” or simply a “replicon,” in particular “self-replicating RNA” or “self-amplifying RNA.” In one particularly preferred embodiment, the replicon or self-replicating RNA is derived from or comprises elements derived from a single-stranded (ss) RNA virus, in particular a positive-stranded ssRNA virus, such as an alphavirus. Alphaviruses are typical representatives of positive-stranded RNA viruses. Alphaviruses replicate in the cytoplasm of infected cells (for review of the alphaviral life cycle see Jose et al., Future Microbiol., 2009, vol. 4, pp. 837-856, which is incorporated herein by reference in its entirety). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124, which is incorporated herein by reference in its entirety). Following infection, i.e. at early stages of the viral life cycle, the (+) stranded genomic RNA directly acts like a messenger RNA for the translation of the open reading frame encoding the non-structural poly-protein (nsP1234).

Alphavirus-derived vectors have been proposed for delivery of foreign genetic information into target cells or target organisms. In simple approaches, a first ORF encodes an alphavirus-derived RNA-dependent RNA polymerase (replicase), which upon translation mediates self-amplification of the RNA. A second ORF encoding alphaviral structural proteins is replaced by an open reading frame encoding a Plasmodium polypeptide construct described herein. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

Features of a non-modified uridine platform may include, for example, one or more of intrinsic adjuvant effect, as well as good tolerability and safety. Features of modified uridine (e.g., pseudouridine) platform may include reduced adjuvant effect, blunted immune innate immune sensor activating capacity and thus good tolerability and safety. Features of self-amplifying platform may include, for example, long duration of protein expression, good tolerability and safety, higher likelihood for efficacy with very low vaccine dose.

The present disclosure provides particular RNA constructs optimized, for example, for improved manufacturability, encapsulation, expression level (and/or timing), etc. Certain components are discussed below, and certain preferred embodiments are exemplified herein.

### V. RNA Delivery Technologies

Provided polyribonucleotides may be delivered for therapeutic applications described herein using any appropriate methods known in the art, including, e.g., delivery as naked RNAs, or delivery mediated by viral and/or non-viral vectors, polymer-based vectors, lipid compositions, nanoparticles (e.g., lipid nanoparticles, polymeric nanoparticles, lipid-polymer hybrid nanoparticles, etc.), and/or peptide-based vectors. See, e.g., Wadhwa et al. “Opportunities and Challenges in the Delivery of mRNA-Based Vaccines” Pharmaceutics (2020) 102 (27 pages), the content of which is incorporated herein by reference, for information on various approaches that may be useful for delivery polyribonucleotides described herein.

In some embodiments, one or more polyribonucleotides can be formulated with lipid nanoparticles for delivery (e.g., administration).

In some embodiments, lipid nanoparticles can be designed to protect polyribonucleotides from extracellular RNases and/or engineered for systemic delivery of the RNA to target cells (e.g., liver cells). In some embodiments, such lipid nanoparticles may be particularly useful to deliver polyribonucleotides when polyribonucleotides are intravenously or intramuscularly administered to a subject.

### A. Lipid Compositions

### 1. Lipids and Lipid-Like Materials

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of a polar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

A “lipid-like material” is a substance that is structurally and/or functionally related to a lipid but may not be considered a lipid in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids.

Specific examples of amphiphilic compounds that may be included in an amphiphilic layer include, but are not limited to, phospholipids, aminolipids and sphingolipids.

Generally, lipids may be divided into eight categories: fatty acids, glycerolipids, glycerophospholipids, sphingolipids, saccharolipids, polyketides (derived from condensation of ketoacyl subunits), sterols and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

Glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are members of a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterols, such as cholesterol and its derivatives, or tocopherol and its derivatives, are important components of membrane lipids, along with the glycerophospholipids and sphingomyelins.

Saccharolipids are compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

Lipids and lipid-like materials may be cationic, anionic or neutral. Neutral lipids or lipid-like materials exist in an uncharged or neutral zwitterionic form at a selected pH.

In some embodiments, suitable lipids or lipid-like materials for use in the present disclosure include those described in WO2020/128031 and US20200163878, the entire contents of each of which are incorporated herein by reference for the purposes described herein.

### 2. Cationic or Cationically Ionizable Lipids or Lipid-Like Materials

In some embodiments cationic or cationically ionizable lipids or lipid-like materials contemplated for use herein include any cationic or cationically ionizable lipids or lipid-like materials which are able to electrostatically bind nucleic acid. In one embodiment, cationic or cationically ionizable lipids or lipid-like materials contemplated for use herein can be associated with nucleic acid, e.g. by forming complexes with the nucleic acid or forming vesicles in which the nucleic acid is enclosed or encapsulated.

Cationic lipids or lipid-like materials are characterized in that they have a net positive charge (e.g., at a relevant pH). Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

In some embodiments, a cationic or cationically ionizable lipid or lipid-like material comprises a head group which includes at least one nitrogen atom (N) which is positive charged or capable of being protonated.

Examples of cationic lipids include, but are not limited to 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-l-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (PAE-DMRIE), N-(4-carboxybenzyl)-N,N-dimethyl-2,3-bis(oleoyloxy)propan-1-aminium (DOBAQ), 2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic lipids for use in the present disclosure include those described in WO2020/128031 and US20200163878, the entire contents of each of which are incorporated herein by reference for the purposes described herein. Further suitable cationic lipids for use in the present disclosure include those described in WO2010/053572 (including Cl 2-200 described at paragraph [00225]) and WO2012/170930, both of which are incorporated herein by reference for the purposes described herein. Additional suitable cationic lipids for use in the present disclosure include HGT4003, HGT5000, HGTS001, HGT5001, HGT5002 (see US20150140070A1, which is incorporated herein by reference in its entirety).

In some embodiments, formulations that are useful for pharmaceutical compositions (e.g., immunogenic compositions, e.g., vaccines) compositions as described herein can comprise at least one cationic lipid. Representative cationic lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120, which is incorporated herein by reference in its entirety).

In some embodiments, amino or cationic lipids useful in accordance with the present disclosure have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention.

In some embodiments, a protonatable lipid has a pKa of the protonatable group in the range of about 4 to about 11, e.g., a pKa of about 5 to about 7.

In some embodiments, a cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of total lipid present in a lipid composition utilized in accordance with the present disclosure.

### 3. Additional Lipids or Lipid-Like Materials

In some embodiments, formulations utilized in accordance with the present disclosure may comprise lipids or lipid-like materials other than cationic or cationically ionizable lipids or lipid-like materials, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. In some embodiments, optimizing a formulation of nucleic acid particles by addition of other hydrophobic moieties, such as cholesterol and lipids, in addition to an ionizable/cationic lipid or lipid-like material may, for example, enhance particle stability and efficacy of nucleic acid delivery.

In some embodiments, a lipid or lipid-like material may be incorporated which may or may not affect the overall charge of particles. In certain embodiments, such lipid or lipid-like material is a non-cationic lipid or lipid-like material.

In some embodiments, a non-cationic lipid may comprise, e.g., one or more anionic lipids and/or neutral lipids. An “anionic lipid” is negatively charged (e.g., at a selected pH).

A “neutral lipid” exists either in an uncharged or neutral zwitterionic form (e.g., at a selected pH). In some embodiments, a formulation comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific exemplary phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain embodiments, a formulation utilized in accordance with the present disclosure includes DSPC or DSPC and cholesterol.

In certain embodiments, formulations utilized in accordance with the present disclosure include both a cationic lipid and an additional (non-cationic) lipid.

In some embodiments, formulations herein include a polymer conjugated lipid such as a pegylated lipid. “Pegylated lipids” comprise both a lipid portion and a polyethylene glycol portion. Pegylated lipids are known in the art.

Without wishing to be bound by theory, the amount of (total) cationic lipid compared to the amount of other lipid(s) in formulation may affect important characteristics, such as charge, particle size, stability, tissue selectivity, and bioactivity of the nucleic acid. In some embodiments, the molar ratio of the at least one cationic lipid to the at least one additional lipid is from about 10:0 to about 1:9, about 4:1 to about 1:2, or about 3:1 to about 1:1.

In some embodiments, a non-cationic lipid, in particular a neutral lipid, (e.g., one or more phospholipids and/or cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in a formulation.

### 4. Lipoplex Particles

In certain embodiments of the present disclosure, the RNA described herein may be present in RNA lipoplex particles.

An “RNA lipoplex particle” contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, a RNA lipoplex particle is a nanoparticle.

In certain embodiments, RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

In some embodiments, RNA lipoplex particles have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

RNA lipoplex particles and compositions comprising RNA lipoplex particles described herein are useful for delivery of RNA to a target tissue after parenteral administration, in particular after intravenous administration. The RNA lipoplex particles may be prepared using liposomes that may be obtained by injecting a solution of the lipids in ethanol into water or a suitable aqueous phase. In one embodiment, the aqueous phase has an acidic pH. In one embodiment, the aqueous phase comprises acetic acid, e.g., in an amount of about 5 mM. Liposomes may be used for preparing RNA lipoplex particles by mixing the liposomes with RNA. In one embodiment, the liposomes and RNA lipoplex particles comprise at least one cationic lipid and at least one additional lipid. In one embodiment, the at least one cationic lipid comprises 1,2-di-O-octadecenyl-3-trimethylammonium propane (DOTMA) and/or 1,2-dioleoyl-3-trimethylammonium-propane (DOTAP). In one embodiment, the at least one additional lipid comprises 1,2-di-(9Z-octadecenoyl)-sn-glycero-3-phosphoethanolamine (DOPE), cholesterol (Chol) and/or 1,2-dioleoyl-sn-glycero-3-phosphocholine (DOPC). In one embodiment, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In one embodiment, the liposomes and RNA lipoplex particles comprise 1,2-di-O-octadecenyl-3-trimethylammonium propane (DOTMA) and 1,2-di-(9Z-octadecenoyl)-sn-glycero-3-phosphoethanolamine (DOPE).

Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages. 5. Lipid Nanoparticles (LNPs)

In some embodiments, nucleic acid such as RNA described herein is administered in the form of lipid nanoparticles (LNPs). In some embodiments, LNPs may comprise any lipid capable of forming a particle to which the one or more nucleic acid molecules are attached, or in which the one or more nucleic acid molecules are encapsulated.

In some embodiments, an LNP comprises one or more cationic lipids, and one or more stabilizing lipids. Stabilizing lipids include neutral lipids and pegylated lipids.

In some embodiments, an LNP comprises a cationic lipid, a neutral lipid, a sterol, a polymer conjugated lipid; and an RNA, encapsulated within or associated with the lipid nanoparticle.

In some embodiments, a neutral lipid is selected from the group consisting of DSPC, DPPC, DMPC, DOPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC.

In some embodiments, a sterol is cholesterol.

In some embodiments, a polymer conjugated lipid is a pegylated lipid. In some embodiments, a pegylated lipid has the following structure:

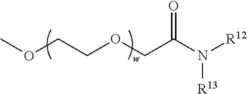

or a pharmaceutically acceptable salt, tautomer or stereoisomer thereof, where:
R¹² and R¹³ are each independently a straight or branched, saturated or unsaturated alkyl chain containing from 10 to 30 carbon atoms, where the alkyl chain is optionally interrupted by one or more ester bonds; and w has a mean value ranging from 30 to 60. In some embodiments, R¹² and R¹³ are each independently straight, saturated alkyl chains containing from 12 to 16 carbon atoms. In some embodiments, w has a mean value ranging from 40 to 55. In some embodiments, the average w is about 45. In some embodiments, R¹² and R¹³ are each independently a straight, saturated alkyl chain containing about 14 carbon atoms, and w has a mean value of about 45.

In some embodiments, a pegylated lipid is DMG-PEG 2000, e.g., having the following structure:

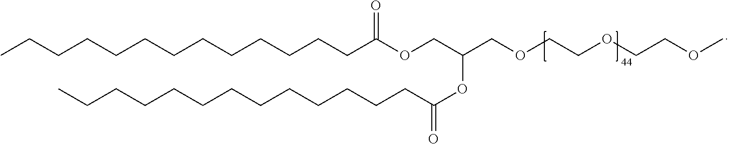

In some embodiments, a cationic lipid component of LNPs has the structure of Formula (III):

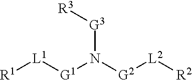

or a pharmaceutically acceptable salt, tautomer, prodrug or stereoisomer thereof, where:
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

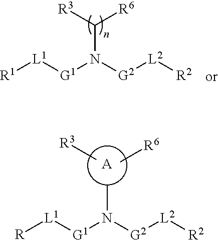

where:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

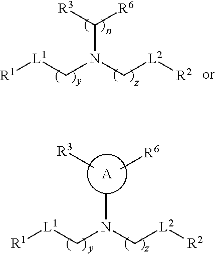

where y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O—or —O(C═O)—. For example, in some embodiments each of L¹ and L² is —(C═O)O—.

In some different embodiments of Formula (III), the lipid has one of the following structures (IIIE) or (IIIF):

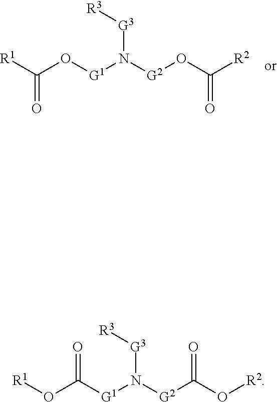

In some of the foregoing embodiments of Formula (III), the lipid has one of the following structures (IIIG), (IIIH), (IIII), or (IIIJ):

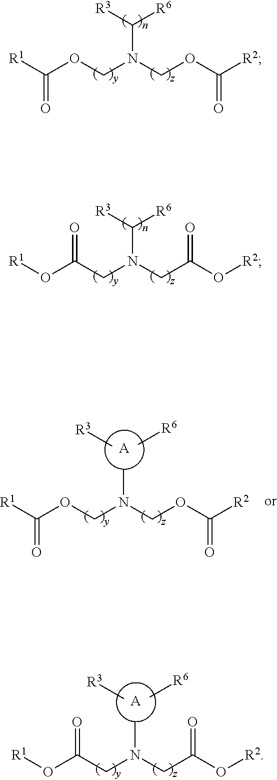

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R6 is H. In other of the foregoing embodiments, R6 is C₁-C₂₄ alkyl. In other embodiments, R6 is OH.

In some embodiments of Formula (III), G³ is unsubstituted. In other embodiments, G³ is substituted. In various different embodiments, G³ is linear C₁-C₂₄ alkylene or linear C₁-C₂₄ alkenylene.

In some other foregoing embodiments of Formula (III), R1 or R2, or both, is C6-C24 alkenyl. For example, in some embodiments, R1 and R2 each, independently have the following structure:

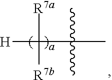

where:
R^{7a} and R^{7b} are, at each occurrence, independently H or C₁-C₁₂ alkyl, and
a is an integer from 2 to 12,
where R^{7a}, R^{7b} and a are each selected such that R1 and R2 each independently comprise from 6 to 20 carbon atoms. For example, in some embodiments a is an integer ranging from 5 to 9 or from 8 to 12.

In some of the foregoing embodiments of Formula (III), at least one occurrence of R⁷ is H. For example, in some embodiments, R7a is H at each occurrence. In other different embodiments of the foregoing, at least one occurrence of R7b is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

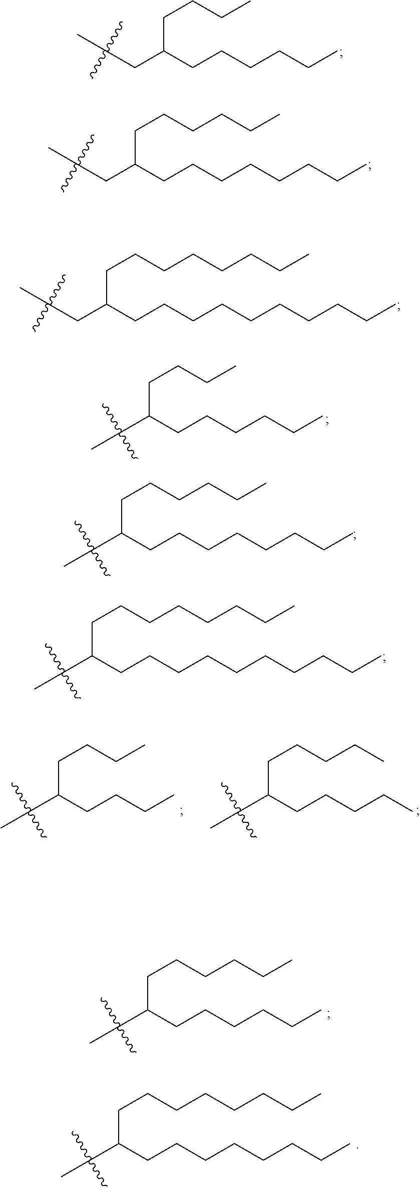

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in in Table 3 below.

TABLE 3
Exemplary Compounds of Formula (III).

| No. | Structure |
|---|---|
| III-1 | 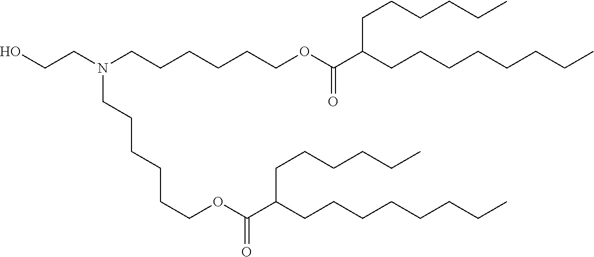 [SMILES: CCCCCCCCC(CCCCCC)C(=O)OCCCCCCN(CCO)CCCCCCOC(=O)C(CCCCCC)CCCCCCCC] |
| III-2 | 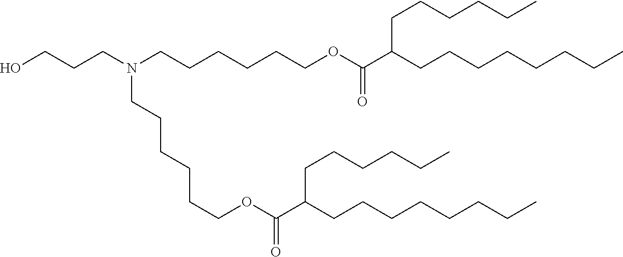 [SMILES: CCCCCCCCC(CCCCCC)C(=O)OCCCCCCN(CCCO)CCCCCCOC(=O)C(CCCCCC)CCCCCCCC] |
| III-3 | 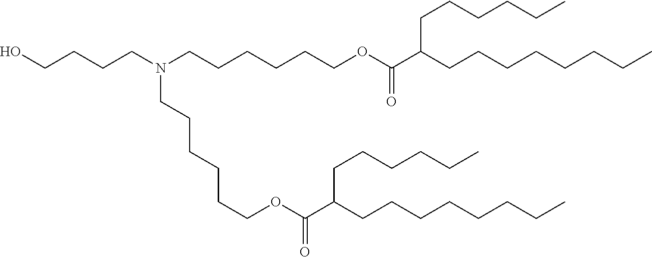 [SMILES: CCCCCCCCC(CCCCCC)C(=O)OCCCCCCN(CCCCO)CCCCCCOC(=O)C(CCCCCC)CCCCCCCC] |
| III-4 | 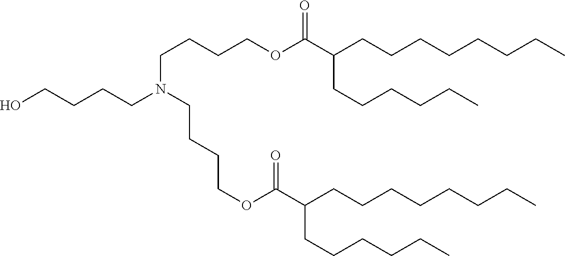 [SMILES: CCCCCCCCC(CCCCCC)C(=O)OCCCCN(CCCCO)CCCCOC(=O)C(CCCCCC)CCCCCCCC] |
| III-5 | 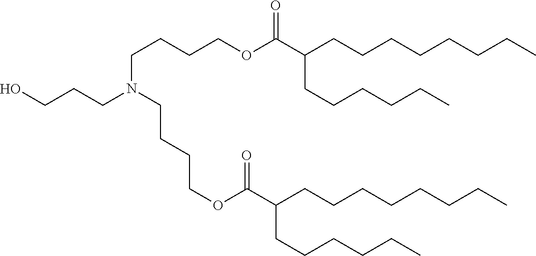 [SMILES: CCCCCCCCC(CCCCCC)C(=O)OCCCCN(CCCO)CCCCOC(=O)C(CCCCCC)CCCCCCCC] |
| III-6 | 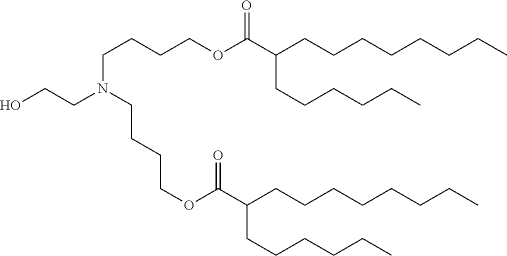 [SMILES: CCCCCCCCC(CCCCCC)C(=O)OCCCCN(CCO)CCCCOC(=O)C(CCCCCC)CCCCCCCC] |
| III-7 | 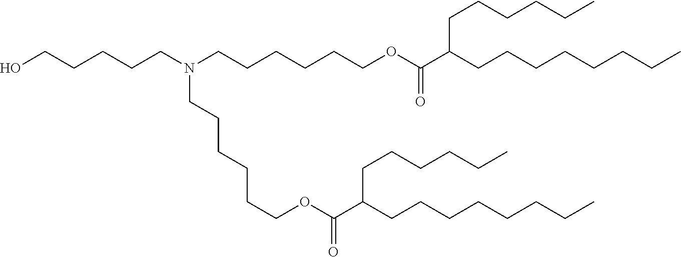 [SMILES: CCCCCCCCC(CCCCCC)C(=O)OCCCCCCN(CCCCCO)CCCCCCOC(=O)C(CCCCCC)CCCCCCCC] |
| III-8 | 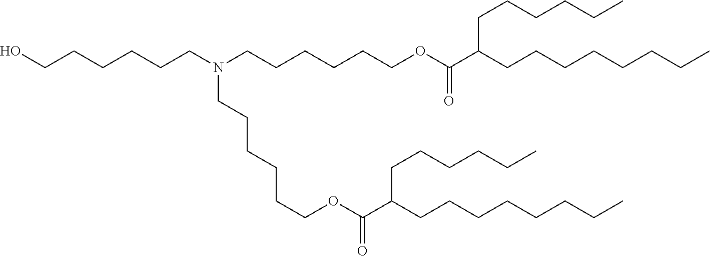 [SMILES: CCCCCCCCC(CCCCCC)C(=O)OCCCCCCN(CCCCCCO)CCCCCCOC(=O)C(CCCCCC)CCCCCCCC] |
| III-9 | 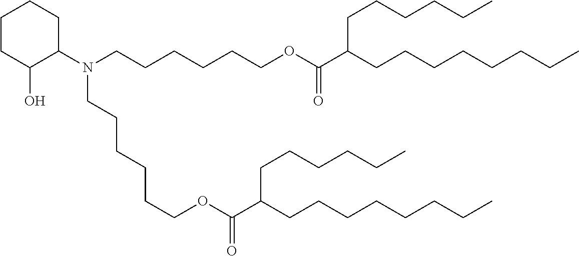 [SMILES: CCCCCCCCC(CCCCCC)C(=O)OCCCCCCN(CCCCCCOC(=O)C(CCCCCC)CCCCCCCC)C1CCCCC1O] |
| III-10 | 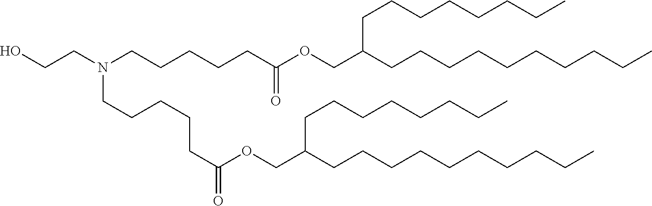 [SMILES: CCCCCCCCCCC(CCCCCCCC)COC(=O)CCCCCN(CCO)CCCCCC(=O)OCC(CCCCCCCC)CCCCCCCCCC] |
| III-11 | 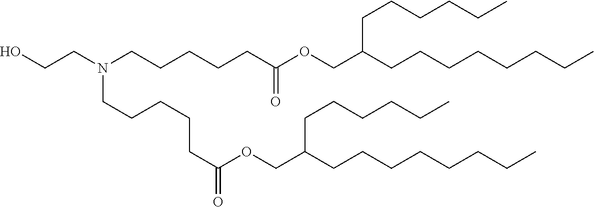 [SMILES: CCCCCCCCC(CCCCCC)COC(=O)CCCCCN(CCO)CCCCCC(=O)OCC(CCCCCC)CCCCCCCC] |
| III-12 | 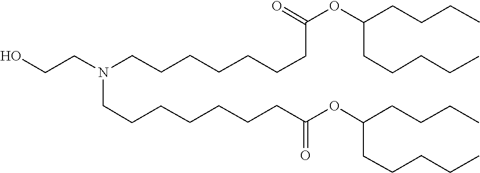 [SMILES: CCCCCC(CCCC)OC(=O)CCCCCCCN(CCO)CCCCCCCC(=O)OC(CCCC)CCCCC] |
| III-13 | 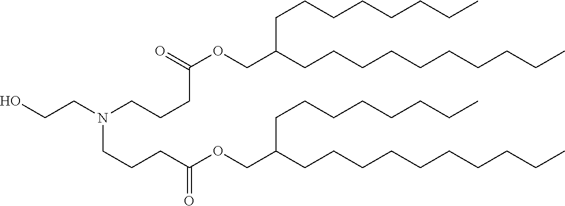 [SMILES: CCCCCCCCCCC(CCCCCCCC)COC(=O)CCCN(CCO)CCCC(=O)OCC(CCCCCCCC)CCCCCCCCCC] |
| III-14 | 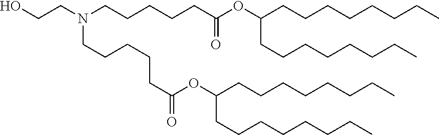 [SMILES: CCCCCCCCC(CCCCCCCC)OC(=O)CCCCCN(CCO)CCCCCC(=O)OC(CCCCCCCC)CCCCCCCC] |
| III-15 | 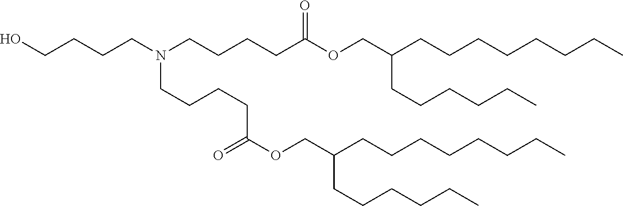 [SMILES: CCCCCCCCC(CCCCCC)COC(=O)CCCCN(CCCCO)CCCCC(=O)OCC(CCCCCC)CCCCCCCC] |
| III-16 | 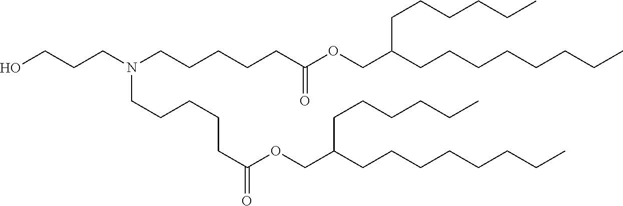 [SMILES: CCCCCCCCC(CCCCCC)COC(=O)CCCCCN(CCCO)CCCCCC(=O)OCC(CCCCCC)CCCCCCCC] |
| III-17 | 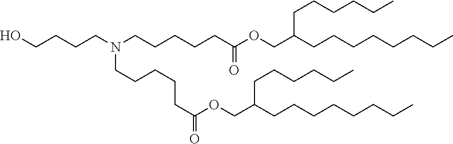 [SMILES: CCCCCCCCC(CCCCCC)COC(=O)CCCCCN(CCCCO)CCCCCC(=O)OCC(CCCCCC)CCCCCCCC] |
| III-18 | 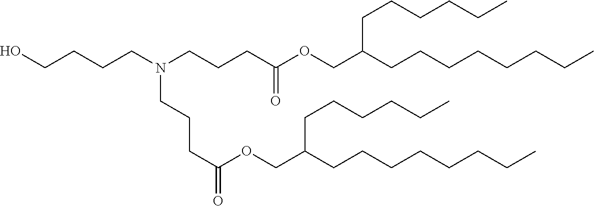 [SMILES: CCCCCCCCC(CCCCCC)COC(=O)CCCN(CCCCO)CCCC(=O)OCC(CCCCCC)CCCCCCCC] |
| III-19 | 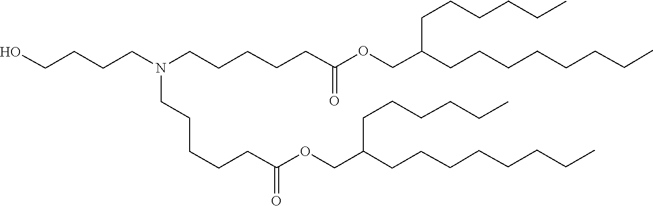 [SMILES: CCCCCCCCC(CCCCCC)COC(=O)CCCCCN(CCCCO)CCCCCC(=O)OCC(CCCCCC)CCCCCCCC] |
| III-20 | 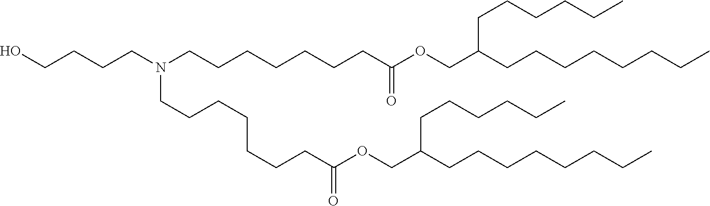 [SMILES: CCCCCCCCC(CCCCCC)COC(=O)CCCCCCCN(CCCCO)CCCCCCCC(=O)OCC(CCCCCC)CCCCCCCC] |
| III-21 | 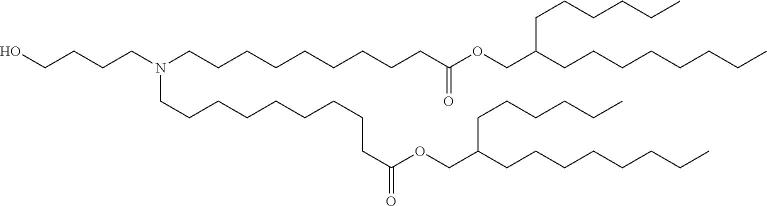 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 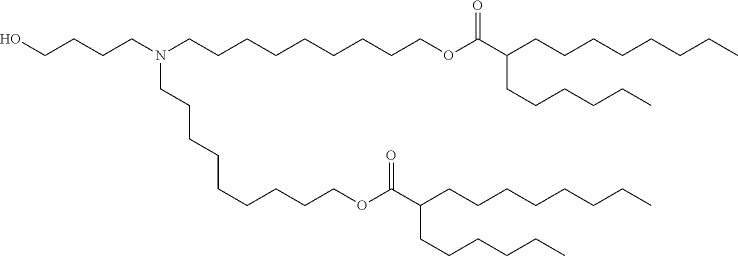 [SMILES: CCCCCCCCC(CCCCCC)C(=O)OCCCCCCCCCN(CCCCO)CCCCCCCCCOC(=O)C(CCCCCC)CCCCCCCC] |
| III-23 | 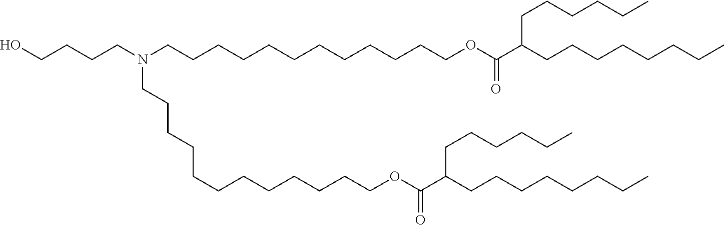 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 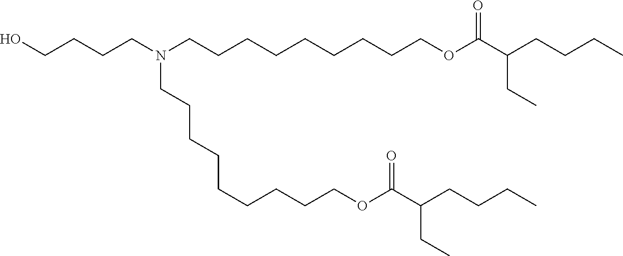 [SMILES: CCCCC(CC)C(=O)OCCCCCCCCCN(CCCCO)CCCCCCCCCOC(=O)C(CC)CCCC] |
| III-25 | 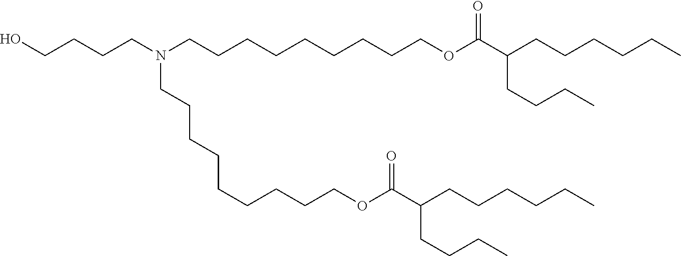 [SMILES: CCCCCCC(CCCC)C(=O)OCCCCCCCCCN(CCCCO)CCCCCCCCCOC(=O)C(CCCC)CCCCCC] |
| III-26 | 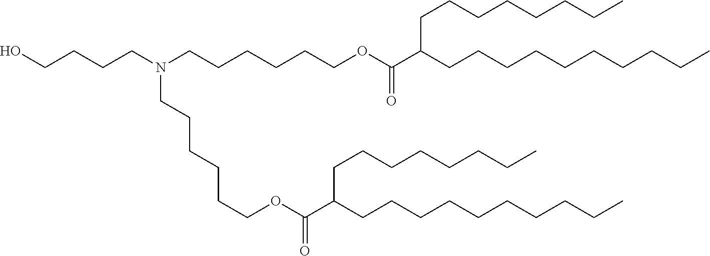 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 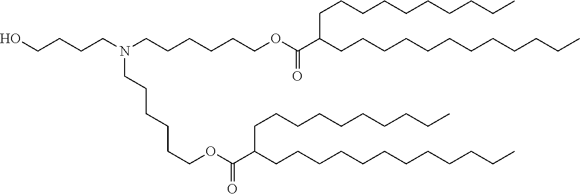 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 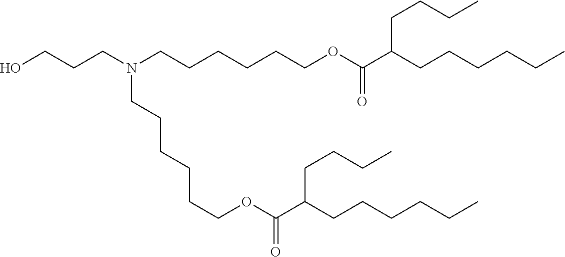 [SMILES: CCCCCCC(CCCC)C(=O)OCCCCCCN(CCCO)CCCCCCOC(=O)C(CCCC)CCCCCC] |
| III-29 | 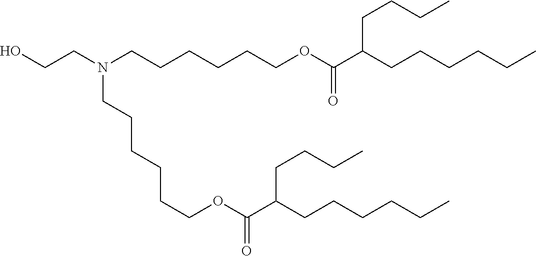 [SMILES: CCCCCCC(CCCC)C(=O)OCCCCCCN(CCO)CCCCCCOC(=O)C(CCCC)CCCCCC] |
| III-30 | 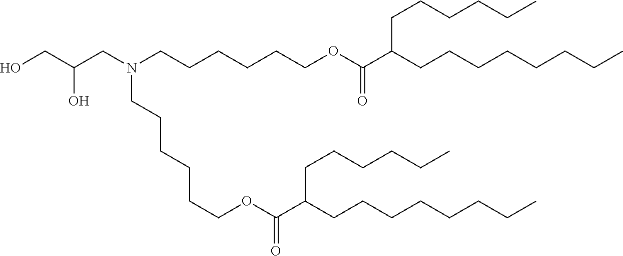 [SMILES: CCCCCCCCC(CCCCCC)C(=O)OCCCCCCN(CCCCCCOC(=O)C(CCCCCC)CCCCCCCC)CC(O)CO] |
| III-31 | 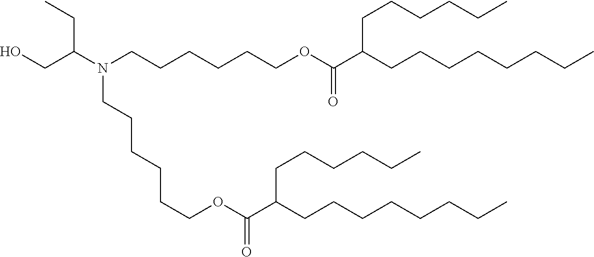 [SMILES: CCCCCCCCC(CCCCCC)C(=O)OCCCCCCN(CCCCCCOC(=O)C(CCCCCC)CCCCCCCC)C(CC)CO] |
| III-32 | 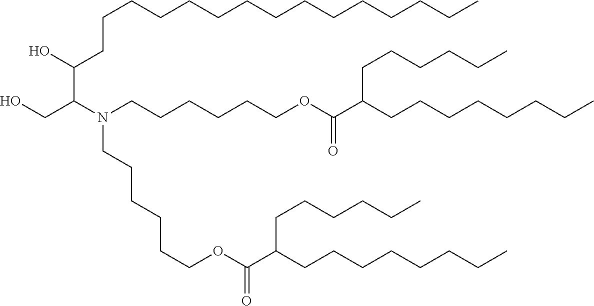 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 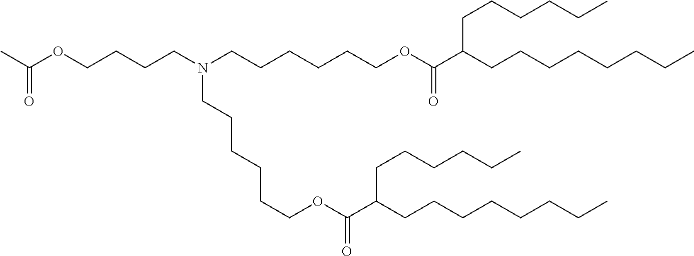 [SMILES: CCCCCCCCC(CCCCCC)C(=O)OCCCCCCN(CCCCCCOC(=O)C(CCCCCC)CCCCCCCC)CCCCOC(C)=O] |
| III-34 | 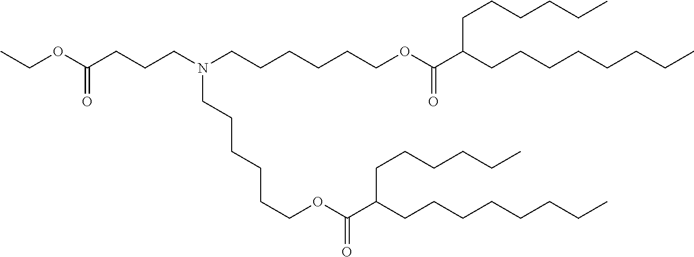 [SMILES: CCCCCCCCC(CCCCCC)C(=O)OCCCCCCN(CCCCCCOC(=O)C(CCCCCC)CCCCCCCC)CCCC(=O)OCC] |
| III-35 | 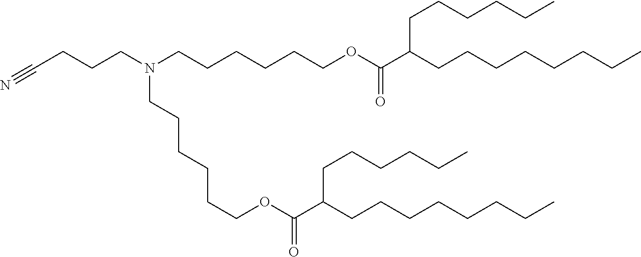 [SMILES: CCCCCCCCC(CCCCCC)C(=O)OCCCCCCN(CCCC#N)CCCCCCOC(=O)C(CCCCCC)CCCCCCCC] |
| III-36 | 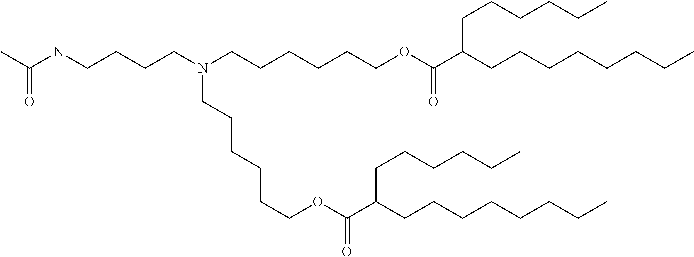 [SMILES: CCCCCCCCC(CCCCCC)C(=O)OCCCCCCN(CCCCCCOC(=O)C(CCCCCC)CCCCCCCC)CCCCNC(C)=O] |

In various different embodiments, a cationic lipid has one of the structures set forth in Table 4 below.

TABLE 4
Exemplary Cationic Lipid Structures

| No. | Structure |
|---|---|
| A | 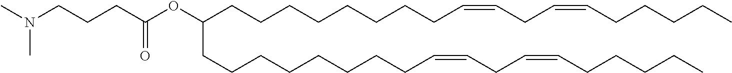 [SMILES: CCCCC/C=C\C/C=C\CCCCCCCCC(CCCCCCCC/C=C\C/C=C\CCCCC)OC(=O)CCCN(C)C] |
| B | 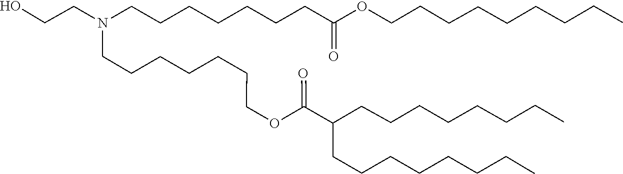 [SMILES: CCCCCCCCCOC(=O)CCCCCCCN(CCO)CCCCCCCOC(=O)C(CCCCCCCC)CCCCCCCC] |
| C | 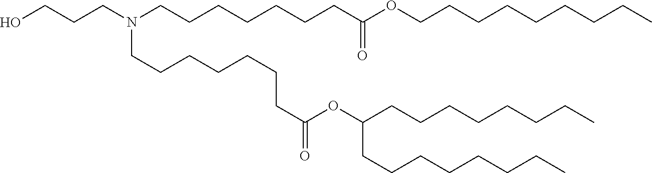 [SMILES: CCCCCCCCCOC(=O)CCCCCCCN(CCCO)CCCCCCCC(=O)OC(CCCCCCCC)CCCCCCCC] |
| D | 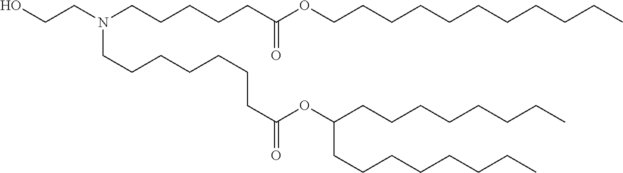 [SMILES: CCCCCCCCCCCOC(=O)CCCCCN(CCO)CCCCCCCC(=O)OC(CCCCCCCC)CCCCCCCC] |
| E | 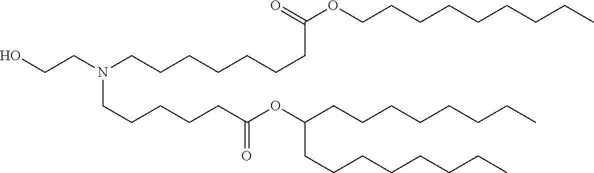 [SMILES: CCCCCCCCCOC(=O)CCCCCCCN(CCO)CCCCCC(=O)OC(CCCCCCCC)CCCCCCCC] |
| F | 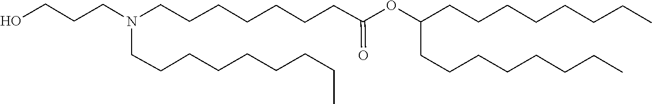 [SMILES: CCCCCCCCCN(CCCO)CCCCCCCC(=O)OC(CCCCCCCC)CCCCCCCC] |

In some embodiments, an LNP comprises a cationic lipid that is an ionizable lipid-like material (lipidoid). In some embodiments, a cationic lipid has the following structure:

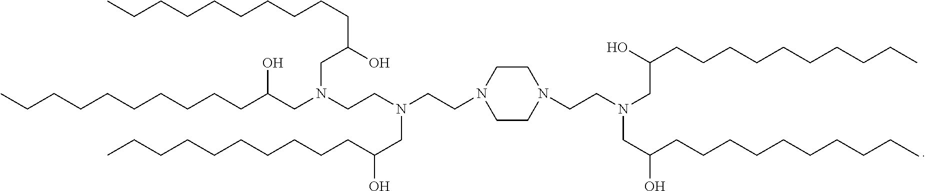 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC ; "."]

In some embodiments, lipid nanoparticles can have an average size (e.g., mean diameter) of about 30 nm to about 150 nm, about 40 nm to about 150 nm, about 50 nm to about 150 nm, about 60 nm to about 130 nm, about 70 nm to about 110 nm, about 70 nm to about 100 nm, about 70 to about 90 nm, or about 70 nm to about 80 nm. In some embodiments, lipid nanoparticles in accordance with the present disclosure can have an average size (e.g., mean diameter) of about 50 nm to about 100 nm. In some embodiments, lipid nanoparticles may have an average size (e.g., mean diameter) of about 50 nm to about 150 nm. In some embodiments, lipid nanoparticles may have an average size (e.g., mean diameter) of about 60 nm to about 120 nm. In some embodiments, lipid nanoparticles in accordance with the present disclosure can have an average size (e.g., mean diameter) of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. The term “average diameter” or “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., 1. Chem. Phys. 57, 1972, pp 4814-4820, ISO 13321, which is herein incorporated by reference). Here “average diameter,” “mean diameter,” “diameter,” or “size” for particles is used synonymously with this value of the Z-average.

In some embodiments, lipid nanoparticles described herein may exhibit a polydispersity index less than about 0.5, less than about 0.4, less than about 0.3, or about 0.2 or less. By way of example, lipid nanoparticles can exhibit a polydispersity index in a range of about 0.1 to about 0.3 or about 0.2 to about 0.3. The “polydispersity index” is preferably calculated based on dynamic light scattering measurements by the so-called cumulant analysis as mentioned in the definition of the “average diameter.” Under certain prerequisites, it can be taken as a measure of the size distribution of an ensemble of ribonucleic acid nanoparticles (e.g., ribonucleic acid nanoparticles).

Lipid nanoparticles described herein can be characterized by an “N/P ratio,” which is the molar ratio of cationic (nitrogen) groups (the “N” in N/P) in the cationic polymer to the anionic (phosphate) groups (the “P” in N/P) in RNA. It is understood that a cationic group is one that is either in cationic form (e.g., N⁺), or one that is ionizable to become cationic. Use of a single number in an N/P ratio (e.g., an N/P ratio of about 5) is intended to refer to that number over 1, e.g., an N/P ratio of about 5 is intended to mean 5:1. In some embodiments, a lipid nanoparticle described herein has an N/P ratio greater than or equal to 5. In some embodiments, a lipid nanoparticle described herein has an N/P ratio that is about 5, 6, 7, 8, 9, or 10. In some embodiments, an N/P ratio for a lipid nanoparticle described herein is from about 10 to about 50. In some embodiments, an N/P ratio for a lipid nanoparticle described herein is from about 10 to about 70. In some embodiments, an N/P ratio for a lipid nanoparticle described herein is from about 10 to about 120.

### B. Exemplary Methods of Making Lipid Nanoparticles

Lipids and lipid nanoparticles comprising nucleic acids and their method of preparation are known in the art, including, e.g., as described in U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2018/081480, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548, the full disclosures each of which are herein incorporated by reference in their entirety for the purposes described herein.

For example, in some embodiments, cationic lipids, neutral lipids (e.g., DSPC, and/or cholesterol) and polymer-conjugated lipids can be solubilized in ethanol at a pre-determined molar ratio (e.g., ones described herein). In some embodiments, lipid nanoparticles (lipid nanoparticle) are prepared at a total lipid to polyribonucleotides weight ratio of approximately 10:1 to 30:1. In some embodiments, such polyribonucleotides can be diluted to 0.2 mg/mL in acetate buffer.

In some embodiments, using an ethanol injection technique, a colloidal lipid dispersion comprising polyribonucleotides can be formed as follows: an ethanol solution comprising lipids, such as cationic lipids, neutral lipids, and polymer-conjugated lipids, is injected into an aqueous solution comprising polyribonucleotides (e.g., ones described herein).

In some embodiments, lipid and polyribonucleotide solutions can be mixed at room temperature by pumping each solution at controlled flow rates into a mixing unit, for example, using piston pumps. In some embodiments, the flow rates of a lipid solution and a RNA solution into a mixing unit are maintained at a ratio of 1:3. Upon mixing, nucleic acid-lipid particles are formed as the ethanolic lipid solution is diluted with aqueous polyribonucleotides. The lipid solubility is decreased, while cationic lipids bearing a positive charge interact with the negatively charged RNA.

In some embodiments, a solution comprising RNA-encapsulated lipid nanoparticles can be processed by one or more of concentration adjustment, buffer exchange, formulation, and/or filtration.

In some embodiments, RNA-encapsulated lipid nanoparticles can be processed through filtration.

In some embodiments, particle size and/or internal structure of lipid nanoparticles (with or without RNAs) may be monitored by appropriate techniques such as, e.g., small-angle X-ray scattering (SAXS) and/or transmission electron cryomicroscopy (CryoTEM).

### VI. Pharmaceutical Compositions

The present disclosure provides compositions, e.g., pharmaceutical compositions comprising one or more polyribonucleotides described herein. Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

General considerations in the formulation and/or manufacture of pharmaceutical agents may be found, for example, in Remington: The Science and Practice of Pharmacy 21st ed., Lippincott Williams & Wilkins, 2005 (incorporated herein by reference).

In some embodiments, pharmaceutical compositions provided herein may be formulated with one or more pharmaceutically acceptable carriers or diluents as well as any other known adjuvants and excipients in accordance with conventional techniques such as those disclosed in Remington: The Science and Practice of Pharmacy 21st ed., Lippincott Williams & Wilkins, 2005 (incorporated herein by reference).

Pharmaceutical compositions described herein can be administered by appropriate methods known in the art. As will be appreciated by a skilled artisan, the route and/or mode of administration may depend on a number of factors, including, e.g., but not limited to stability and/or pharmacokinetics and/or pharmacodynamics of pharmaceutical compositions described herein.

In some embodiments, pharmaceutical compositions described herein are formulated for parenteral administration, which includes modes of administration other than enteral and topical administration, usually by injection, and includes, without limitation, intravenous, intramuscular, intraarterial, intradermal, subcutaneous, subcuticular, or intraarticular injection and infusion. In preferred embodiments, pharmaceutical compositions described herein are formulated for intravenous, intramuscular, or subcutaneous administration. In particularly preferred embodiments, pharmaceutical compositions described herein are formulated for intramuscular administration.

In some embodiments, pharmaceutical compositions described herein are formulated for intravenous administration. In some embodiments, pharmaceutically acceptable excipients that may be useful for intravenous administration include sterile aqueous solutions or dispersions and sterile powders for preparation of sterile injectable solutions or dispersions.

Therapeutic compositions typically must be sterile and stable under the conditions of manufacture and storage. The composition can be formulated as a solution, microemulsion, lipid nanoparticles, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. Proper fluidity can be maintained, for example, by the use of surfactants. In many cases, it will be preferable to include isotonic agents, for example, sugars, polyalcohols such as mannitol, sorbitol, or sodium chloride in the composition. In some embodiments, prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization and/or microfiltration. In some embodiments, pharmaceutical compositions can be prepared as described herein and/or methods known in the art. In some embodiments, a pharmaceutical composition includes ALC-0315; ALC-0159; DSPC; Cholesterol; Sucrose; NaCl; KCl; Na₂HPO₄; KH₂PO₄; Water for injection. In some embodiments, normal saline (isotonic 0.9% NaCl) is used as diluent.

These compositions may also contain adjuvants such as preservatives, wetting agents, emulsifying agents and dispersing agents. Prevention of the presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into pharmaceutical compositions described herein. In addition, prolonged absorption of the injectable pharmaceutical form may be brought about by the inclusion of agents which delay absorption such as aluminum monostearate and gelatin.

Formulations of pharmaceutical compositions described herein may be prepared by any method known or hereafter developed in the art of pharmacology. In general, such preparatory methods include the step of bringing active ingredient(s) into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of at least one RNA product produced using a system and/or method described herein.

Relative amounts of polyribonucleotides encapsulated in lipid nanoparticles, a pharmaceutically acceptable excipient, and/or any additional ingredients in a pharmaceutical composition can vary, depending upon the subject to be treated, target cells, diseases or disorders, and may also further depend upon the route by which the composition is to be administered.

In some embodiments, pharmaceutical compositions described herein are formulated into pharmaceutically acceptable dosage forms by conventional methods known to those of skill in the art. Actual dosage levels of the active ingredients (e.g., polyribonucleotides encapsulated in lipid nanoparticles) in the pharmaceutical compositions described herein may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician having ordinary skill in the art can readily determine and prescribe the effective amount of the pharmaceutical composition required. For example, a physician could start doses of active ingredients (e.g., polyribonucleotides encapsulated in lipid nanoparticles) employed in the pharmaceutical composition at levels lower than that required in order to achieve the desired therapeutic effect and gradually increase the dosage until the desired effect is achieved.

In some embodiments, a pharmaceutical composition is formulated (e.g., but not limited to, for intravenous, intramuscular, or subcutaneous administration) to deliver a dose of about 5 mg RNA/kg.

In some embodiments, a pharmaceutical composition described herein may further comprise one or more additives, for example, in some embodiments that may enhance stability of such a composition under certain conditions. Examples of additives may include but are not limited to salts, buffer substances, preservatives, and carriers. For example, in some embodiments, a pharmaceutical composition may further comprise a cryoprotectant (e.g., sucrose) and/or an aqueous buffered solution, which may in some embodiments include one or more salts, including, e.g., alkali metal salts or alkaline earth metal salts such as, e.g., sodium salts, potassium salts, and/or calcium salts.

In some embodiments, a pharmaceutical composition provided herein is a preservative-free, sterile RNA-lipid nanoparticle dispersion in an aqueous buffer for intravenous or intramuscular administration.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

### VII. Patient Populations

In some aspects, technologies of the present disclosure are used for therapeutic and/or prophylactic purposes. In some embodiments, technologies of the present disclosure are used in the treatment and/or prophylactic of an infection with a Plasmodium parasite. Prophylactic purposes of the present disclosure comprise pre-exposure prophylaxis and/or post-exposure prophylaxis. In some such embodiments, a Plasmodium parasite is, for example, Plasmodium falciparum, Plasmodium knowlesi, Plasmodium ovale, Plasmodium simiovale, Plasmodium vivax, Plasmodium malariae, Plasmodium ovale curtisi, Plasmodium ovale wallikeri, and/or Plasmodium berghei.

In some embodiments, technologies of the present disclosure are used in the treatment and/or prophylaxis of a disorder related to such an infection. A disordered related to such an infection comprises, for example, a typical symptom and/or a complication of a malaria infection.

In some embodiments, provided compositions (e.g., that are or comprise Plasmodium antigens) may be useful to detect and/or characterize one or more features of an anti-malarial immune response (e.g., by detecting binding to a provided antigen by serum from an infected subject).

In some embodiments, provided compositions (e.g., that are or comprise Plasmodium antigens) are useful to raise antibodies to one or more epitopes included therein; such antibodies may themselves be useful, for example for detection or treatment of Plasmodium parasite(s) or infection thereby.

The present disclosure provides use of encoding nucleic acids (e.g., DNA or RNA) to produce encoded antigens and/or use of DNA constructs to produce RNA.

In some embodiments, technologies of the present disclosure are utilized in a non-limited subject population; in some embodiments, technologies of the present disclosure are utilized in particular subject populations.

In some embodiments, a subject population comprises an adult population. In some embodiments, an adult population comprises subjects between the ages of about 18 years and about 60 years of age (e.g., about 20, 25, 30, 35, 40, 45, 50, 55, or 60 years of age). In some embodiments, a subject population comprises an adult population. In some embodiments, an adult population comprises subjects between the ages of about 19 years and about 60 years of age (e.g., about 20, 25, 30, 35, 40, 45, 50, 55, or 60 years of age). In some embodiments, an adult population comprises subjects between the ages of about 18 years and about 55 years of age (e.g., about 20, 25, 30, 35, 40, 45, 50, 55, or 60 years of age).

In some embodiments, a subject population comprises an elderly population. In some embodiments, an elderly population comprises subjects of about 60 years of age, about 70 years of age, or older (e.g., about 65, 70, 75, 80, 85, 90, 95, or 100 years of age).

In some embodiments, a subject population comprises a pediatric population. In some embodiments, a pediatric population comprises subjects approximately 18 years old or younger. In some such embodiments, a pediatric population comprises subjects between the ages of about 1 year and about 18 years (e.g., 1, 2, 3, 4, 5, 6, 7, 8, 9, 10, 11, 12, 13, 14, 15, 16, 17, or 18 years of age).

In some embodiments, a subject population comprises a newborn population. In some embodiments, a newborn population comprises subjects about 12 months or younger (e.g., 12, 11, 10, 9, 8, 7, 6, 5, 4, 3, 2, 1 months or younger). In some embodiments, subject populations to be treated with technologies described herein include infants (e.g., about 12 months or younger) whose mothers did not receive such technologies described herein during pregnancy. In some embodiments, subject populations to be treated with technologies described herein may include pregnant women; in some embodiments, infants whose mothers were treated with disclosed technologies during pregnancy (e.g., who received at least one dose, or alternatively only who received both doses), are not vaccinated during the first weeks, months, or even years (e.g., 1, 2, 3, 4, 5, 6, 7, 8 weeks or more, or 1, 2, 3, 4, 5, 6, 7, 8, 9, 10, 11, 12, 13, 14, 15, 16, 17, 18, 19, 20, 21, 22, 23, 24 months or more, or 1, 2, 3, 4, 5 years or more) post-birth. Alternatively or additionally, in some embodiments, infants whose mothers were treated with disclosed technologies during pregnancy (e.g., who received at least one dose, or alternatively only who received both doses), receive reduced treated with disclosed technologies (e.g., lower doses and/or smaller numbers of administrations—e.g., boosters—and/or lower total exposure over a given period of time) after birth, for example during the first weeks, months, or even years (e.g., 1, 2, 3, 4, 5, 6, 7, 8 weeks or more, or 1, 2, 3, 4, 5, 6, 7, 8, 9, 10, 11, 12, 13, 14, 15, 16, 17, 18, 19, 20, 21, 22, 23, 24 months or more, or 1, 2, 3, 4, 5 years or more) post-birth or may need reduced vaccination (e.g., lower doses and/or smaller numbers of administrations—e.g., boosters—over a given period of time), In some embodiments, compositions as provided herein are administered to subject populations that do not include pregnant women.

In some embodiments, a subject population is or comprises children aged 6 weeks to up to 17 months of age.

In some embodiments, a subject has a body mass index over 15 kg/m² and under 40 kg/m². In some embodiments, a subject has a body mass index over 18.5 kg/m² and under 35 kg/m. In some embodiments, a subject's body mass index is determined at an initial visit with a health professional. In some embodiments, a subject's body mass index is determined when a first dose of a pharmaceutical composition (e.g., immunogenic composition, e.g., vaccine) as described herein is administered.

In some embodiments, a subject weighs at least 40 kg. In some embodiments, a subject weighs at least 45 kg. In some embodiments, a subject's weight is determined at an initial visit with a health professional. In some embodiments, a subject's weight is determined when a first dose of a pharmaceutical composition (e.g., immunogenic composition, e.g., vaccine) as described herein is administered.

In some embodiments, a subject has a body mass index over 15 kg/m² and under 40 kg/m² and weighs at least 40 kg. In some embodiments, a subject has a body mass index over 18.5 kg/m² and under 35 kg/m2 and weighs at least 45 kg. In some embodiments, a subject's body mass index and weight are determined at an initial visit with a health professional. In some embodiments, a subject's body mass index and weight are determined when a first dose of a pharmaceutical composition (e.g., immunogenic composition, e.g., vaccine) as described herein is administered.

In some embodiments, a subject population comprises a population with a high risk of infection (e.g., malaria). In some such embodiments, a population may be deemed to have a high risk of infection due to a local epidemic or a global pandemic. In some such embodiments, a population may be deemed to have a high risk of infection due to a subject population's geographic area. In some embodiments, a subject population comprises subjects that have been exposed to infection (e.g., malaria). In some embodiments, a subject is malaria naive, e.g., has not experienced a prior malarial infection. In some embodiments, a subject previously has been exposed to malaria (e.g., Plasmodium falciparum). Previous exposure to malaria does not encompass a prior vaccination with a malarial parasite or a component thereof. In some embodiments, a subject previously has been infected with malaria (e.g., Plasmodium falciparum).

In some embodiments, a subject has not traveled to a malaria-endemic region within the 3 months, 4 months, 5 months, 6 months or 1 year prior to receiving a first dose of a polyribonucleotide, RNA construct, or composition (e.g., pharmaceutical composition) described herein. In some embodiments, a subject has traveled to a malaria-endemic region within the 3 months, 4 months, 5 months, 6 months or 1 year prior to receiving a first dose. In some embodiments, a subject intends to travel to a malaria-endemic region within 3 months, 4 months, 5 months, 6 months or 1 year after receiving a first dose. In some embodiments, a subject resides in a malaria-endemic region. A malaria-endemic region is defined by the Center for Disease Control and Prevention. In some embodiments, a malaria-endemic region is defined by the Center for Disease Control and Prevention at www.cdc.gov/malaria/travelers/country_table/a.html, as of Jul. 24, 2023. In some embodiments, a malaria-endemic region includes all or a portion of Afghanistan, Angola, Bangladesh, Bhutan, Bolivia, Botswana, Brazil, Burma, Cameroon, Cambodia, Central African Republic, Chad, Colombia, Comoros, Congo, Costa Rica, Cote d'Ivoire, Democratic Republic of the Congo, Djibouti, Dominican Republic, Ecuador, Equatorial Guinea, Eritrea, Eswatini, Ethopia, French Guiana, Gabon, Gambia, Ghana, Guatemala, Guinea, Guinea-Bissau, Guyana, Haiti, Honduras, India, Indonesia, Kenya, Laos, Liberia, Madagascar, Malawi, Mali, Mauritania, Mexico, Mozambique, Namibia, Nepal, Nicaragua, Niger, Nigeria, North Korea, Pakistan, Panama, Papua New Guinea, Peru, Philippines, Rwanda, São Tomé and Principe, Saudia Arabia, Senegal, Sierra Leone, Solomon Islands, Somalia, South Africa, South Korea, South Sudan, Sudan, Suriname, Tanzania, Thailand, Togo, Uganda, Vanuatu, Venezuela, Vietnam, Yemen, Zambia, and/or Zimbabwe.

In some embodiments, where a subject population is or includes pregnant women, provided technologies offer a particular advantage of interrupting malaria's transmission cycle, including, for example, in some embodiments, by reducing or eliminating transmission from pregnant mothers to their fetuses.

In some embodiments, a subject population is or comprises immunocompromised individuals. In some embodiments, a subject population does not include immunocompromised individuals.

In some embodiments, a provided pharmaceutical composition (e.g., immunogenic composition, e.g., vaccine) may be administered in combination with (i.e., so that subject(s) are simultaneously exposed to both) another pharmaceutical composition (e.g., immunogenic composition, e.g., vaccine) or therapeutic intervention, e.g., to treat or prevent malaria or another disease, disorder, or condition.

In some embodiments, a provided pharmaceutical composition (e.g., immunogenic composition, e.g., vaccine) may be administered with a protein vaccine, a DNA vaccine, an RNA vaccine, a cellular vaccine, a conjugate vaccine, etc. In some embodiments, one or more doses of a provided pharmaceutical composition (e.g., immunogenic composition, e.g., vaccine) may be administered together with (e.g., in a single visit) another vaccine or other therapy.

In some embodiments, a provided pharmaceutical composition (e.g., immunogenic composition, e.g., vaccine) may be administered to subjects who have been exposed, or expect they have been exposed, to malaria. In some embodiments, a provided pharmaceutical composition (e.g., immunogenic composition, e.g., vaccine) may be administered to subjects who do not have symptoms of malarial infection.

### VIII. Treatment Methods

In some embodiments, technologies of the present disclosure may be administered to subjects according to a particular dosing regimen. In some embodiments, a dosing regimen may involve a single administration; in some embodiments, a dosing regimen may comprise one or more “booster” administrations after the initial administration. In some embodiments, initial and boost doses are the same amount; in some embodiments they differ. In some embodiments, two or more booster doses are administered. In some embodiments, a plurality of doses are administered at regular intervals. In some embodiments, periods of time between doses become longer. In some embodiments, one or more subsequent doses is administered if a particular clinical (e.g., reduction in neutralizing antibody levels) or situational (e.g., local development of a new strain) even arises or is detected.

In some embodiments, administered pharmaceutical compositions (e.g., immunogenic compositions, e.g., vaccines) comprising RNA constructs that encode Plasmodium polypeptide constructs are administered in RNA doses of from about 0.1 μg to about 300 μg, about 0.5 μg to about 200 μg, or about 1 μg to about 100 μg, such as about 1 μg, about 3 μg, about 10 μg, about 30 μg, about 50 μg, about 70 μg, or about 100 μg. In some embodiments, administered pharmaceutical compositions (e.g., immunogenic compositions, e.g., vaccines) comprising RNA constructs that encode Plasmodium polypeptide constructs are administered in RNA doses of 10 μg or less, 30 μg or less, 50 μg or less, 70 μg or less, or 100 μg or less. In some embodiments, an saRNA construct is administered at a lower dose (e.g., 2, 4, 5, 10 fold or more lower) than a modRNA or uRNA construct.

In some embodiments, a first booster dose is administered within a about six months of the initial dose, and preferably within about 5, 4, 3, 2, or 1 months. In some embodiments, a first booster dose is administered in a time period that begins about 1, 2, 3, or 4 weeks after the first dose, and ends about 2, 3, 4, 5, 6, 7, 8, 9, 10, 11, 12 weeks of the first dose (e.g., between about 1 and about 12 weeks after the first dose, or between about 2 or 3 weeks and about 5 and 6 weeks after the first dose, or about 3 weeks or about 4 weeks after the first dose).

In some embodiments, a plurality of booster doses (e.g., 2, 3, or 4) doses are administered within 6 months of the first dose, or within 12 months of the first dose.

In some embodiments, 3 doses or fewer are required to achieve effective vaccination (e.g., greater than 60%, and in some embodiments greater than about 70%, about 75%, about 80%, about 85%, about 90% or more) reduction in risk of infection, or of serious disease. In some embodiments, not more than two doses are required. In some embodiments, a single dose is sufficient. In some embodiments, an RNA dose is about 60 μg or lower, 50 μg or lower, 40 μg or lower, 30 μg or lower, 20 μg or lower, 10 μg or lower, 5 μg or lower, 2.5 μg or lower, or 1 μg or lower. In some embodiments, an RNA dose is about 0.25 μg, at least 0.5 μg, at least 1 μg, at least 2 μg, at least 3 μg, at least 4 μg, at least 5 μg, at least 10 μg, at least 20 μg, at least 30 μg, or at least 40 μg. In some embodiments, an RNA dose is about 0.25 μg to 60 μg, 0.5 μg to 55 μg, 1 μg to 50 μg, 5 μg to 40 μg, or 10 μg to 30 μg may be administered per dose. In some embodiments, an RNA dose is about 30 μg. In some embodiments, at least two such doses are administered. For example, a second dose may be administered about 21 days following administration of the first dose. In some embodiments, a first booster dose is administered about one month after an initial dose. In some such embodiments, at least one further booster is administered at one-month interval(s). In some embodiments, after 2 or 3 boosters, a longer interval is introduced and no further booster is administered for at least 6, 9, 12, 18, 24, or more months. In some embodiments, a single further booster is administered after about 18 months. In some embodiments, no further booster is required unless, for example, a material change in clinical or environmental situation is observed.

In some embodiments, one or more outcomes may be assessed following administration of one or more doses of a pharmaceutical composition provided herein. Exemplary outcomes include, but are not limited to: local reaction at the injection site (e.g., pain, erythema/redness, induration/swelling, etc.), frequency of solicited local reactions at the injection site (e.g., pain, erythema/redness, induration/swelling, etc.), systemic reactions (e.g., vomiting, diarrhea, headache, fatigue, muscle/joint pain, fever, etc.), frequency of solicited systemic reactions (e.g., vomiting, diarrhea, headache, fatigue, muscle/joint pain, fever, etc.), adverse events, medically attended adverse events, severe adverse events, frequency of subjects with at least one adverse event, frequency of subjects with at least one medically attended adverse events, frequency of subjects with at least one severe adverse events, number of subjects protected from blood stage parasitemia, proportion of subjects protected from blood stage parasitemia, and combinations thereof.

In some embodiments, blood stage parasitemia can be assessed by PCR, e.g., qPCR.

In some embodiments, statistics on antibody levels at assessed time points can be obtained following administration of one or more doses of a pharmaceutical composition provided herein. Time points can include at the time of a first dose, the time of a second dose, the time of a third dose, following a known or suspected malaria exposure, or following a known or suspected malaria infection.

### IX. Methods of Manufacture

Individual polyribonucleotides can be produced by methods known in the art. For example, in some embodiments, polyribonucleotides can be produced by in vitro transcription, for example, using a DNA template. A plasmid DNA used as a template for in vitro transcription to generate a polyribonucleotide described herein is also within the scope of the present disclosure.

A DNA template is used for in vitro RNA synthesis in the presence of an appropriate RNA polymerase (e.g., a recombinant RNA-polymerase such as a T7 RNA-polymerase) with ribonucleotide triphosphates (e.g., ATP, CTP, GTP, UTP). In some embodiments, polyribonucleotides (e.g., ones described herein) can be synthesized in the presence of modified ribonucleotide triphosphates. By way of example only, in some embodiments, pseudouridine (ψ), N1-methyl-pseudouridine (m1ψ), or 5-methyl-uridine (m5U) can be used to replace uridine triphosphate (UTP). In some embodiments, pseudouridine (ψ) can be used to replace uridine triphosphate (UTP). In some embodiments, N1-methyl-pseudouridine (m1ψ) can be used to replace uridine triphosphate (UTP). In some embodiments, 5-methyl-uridine (m5U) can be used to replace uridine triphosphate (UTP).

As will be clear to those skilled in the art, during in vitro transcription, an RNA polymerase (e.g., as described and/or utilized herein) typically traverses at least a portion of a single-stranded DNA template in the 3′-5′ direction to produce a single-stranded complementary RNA in the 5′-3′ direction.

In some embodiments where a polyribonucleotide comprises a polyA tail, one of those skill in the art will appreciate that such a polyA tail may be encoded in a DNA template, e.g., by using an appropriately tailed PCR primer, or it can be added to a polyribonucleotide after in vitro transcription, e.g., by enzymatic treatment (e.g., using a poly(A) polymerase such as an E. coli Poly(A) polymerase). Suitable poly(A) tails are described herein above. For example, in some embodiments, a poly(A) tail comprises a nucleotide sequence of: AAAAAAAAAAAAAAAAAAAAAAAAAAAAAAGCAUAUGACUAAAAAAAAAAAAAAAAAAAAAAAAAAAAAAAAAAAAAAAAAAAAAA AAAAAAAAAAAAAAAAAAAAAAAA (SEQ ID NO: 66). In some embodiments, a poly(A) tail comprises a plurality of A residues interrupted by a linker. In some embodiments, a linker comprises the nucleotide sequence GCATATGAC.

In some embodiments, those skilled in the art will appreciate that addition of a 5′ cap to an RNA (e.g., mRNA) can facilitate recognition and attachment of the RNA to a ribosome to initiate translation and enhances translation efficiency. Those skilled in the art will also appreciate that a 5′ cap can also protect an RNA product from 5′ exonuclease mediated degradation and thus increases half-life. Methods for capping are known in the art; one of ordinary skill in the art will appreciate that in some embodiments, capping may be performed after in vitro transcription in the presence of a capping system (e.g., an enzyme-based capping system such as, e.g., capping enzymes of vaccinia virus). In some embodiments, a cap may be introduced during in vitro transcription, along with a plurality of ribonucleotide triphosphates such that a cap is incorporated into a polyribonucleotide during transcription (also known as co-transcriptional capping). In some embodiments, a GTP fed-batch procedure with multiple additions in the course of the reaction may be used to maintain a low concentration of GTP in order to effectively cap the RNA. Suitable 5′ cap are described herein above. For example, in some embodiments, a 5′ cap comprises m7(3′OMeG)(5′)ppp(5′)(2′OMeA)pG.

Following RNA transcription, a DNA template is digested. In some embodiments, digestion can be achieved with the use of DNase I under appropriate conditions.

In some embodiments, in-vitro transcribed polyribonucleotides may be provided in a buffered solution, for example, in a buffer such as HEPES, a phosphate buffer solution, a citrate buffer solution, an acetate buffer solution; in some embodiments, such solution may be buffered to a pH within a range of, for example, about 6.5 to about 7.5; in some embodiments approximately 7.0. In some embodiments, production of polyribonucleotides may further include one or more of the following steps: purification, mixing, filtration, and/or filling.

In some embodiments, polyribonucleotides can be purified (e.g., in some embodiments after in vitro transcription reaction), for example, to remove components utilized or formed in the course of the production, like, e.g., proteins, DNA fragments, and/or or nucleotides. Various nucleic acid purifications that are known in the art can be used in accordance with the present disclosure. Certain purification steps may be or include, for example, one or more of precipitation, column chromatography (including, e.g., but not limited to anionic, cationic, hydrophobic interaction chromatography (HIC)), solid substrate-based purification (e.g., magnetic bead-based purification). In some embodiments, polyribonucleotides may be purified using magnetic bead-based purification, which in some embodiments may be or comprise magnetic bead-based chromatography. In some embodiments, polyribonucleotides may be purified using hydrophobic interaction chromatography (HIC) and/or diafiltration. In some embodiments, polyribonucleotides may be purified using HIC followed by diafiltration.

In some embodiments, dsRNA may be obtained as side product during in vitro transcription. In some such embodiments, a second purification step may be performed to remove dsRNA contamination. For example, in some embodiments, cellulose materials (e.g., microcrystalline cellulose) may be used to remove dsRNA contamination, for examples in some embodiments in a chromatographic format. In some embodiments, cellulose materials (e.g., microcrystalline cellulose) can be pretreated to inactivate potential RNase contamination, for example in some embodiments by autoclaving followed by incubation with aqueous basic solution, e.g., NaOH. In some embodiments, cellulose materials may be used to purify polyribonucleotides according to methods described in WO 2017/182524, the entire content of which is incorporated herein by reference.

In some embodiments, a batch of polyribonucleotides may be further processed by one or more steps of filtration and/or concentration. For example, in some embodiments, polyribonucleotide(s), for example, after removal of dsRNA contamination, may be further subject to diafiltration (e.g., in some embodiments by tangential flow filtration), for example, to adjust the concentration of polyribonucleotides to a desirable RNA concentration and/or to exchange buffer to a drug substance buffer.

In some embodiments, polyribonucleotides may be processed through 0.2 μm filtration before they are filled into appropriate containers.

In some embodiments, polyribonucleotides and compositions thereof may be manufactured in accordance with a process as described herein, or as otherwise known in the art.

In some embodiments, polyribonucleotides and compositions thereof may be manufactured at a large scale. For example, in some embodiments, a batch of polyribonucleotides can be manufactured at a scale of greater than 1 g, greater than 2 g, greater than 3 g, greater than 4 g, greater than 5 g, greater than 6 g, greater than 7 g, greater than 8 g, greater than 9 g, greater than 10 g, greater than 15 g, greater than 20 g, or higher.

In some embodiments, RNA quality control may be performed and/or monitored at any time during production process of polyribonucleotides and/or compositions comprising the same. For example, in some embodiments, RNA quality control parameters, including one or more of RNA identity (e.g., sequence, length, and/or RNA natures), RNA integrity, RNA concentration, residual DNA template, and residual dsRNA, may be assessed and/or monitored after each or certain steps of a polyribonucleotide manufacturing process, e.g., after in vitro transcription, and/or each purification step.

In some embodiments, the stability of polyribonucleotides (e.g., produced by in vitro transcription) and/or compositions comprising polyribonucleotides can be assessed under various test storage conditions, for example, at room temperatures vs. fridge or sub-zero temperatures over a period of time (e.g., at least 3 months, at least 6 months, at least 9 months, at least 12 months, or longer). In some embodiments, polyribonucleotides (e.g., ones described herein) and/or compositions thereof may be stored stable at a fridge temperature (e.g., about 4° C. to about 10° C.) for at least 1 month or longer including, at least 2 months, at least 3 months, at least 4 months, at least 5 months, at least 6 months, at least 7 months, at least 8 months, at least 9 months, at least 10 months, at least 11 months, or at least 12 months or longer. In some embodiments, polyribonucleotides (e.g., ones described herein) and/or compositions thereof may be stored stable at a sub-zero temperature (e.g., −20° C. or below) for at least 1 month or longer including, at least 2 months, at least 3 months, at least 4 months, at least 5 months, at least 6 months, at least 7 months, at least 8 months, at least 9 months, at least 10 months, at least 11 months, or at least 12 months or longer. In some embodiments, polyribonucleotides (e.g., ones described herein) and/or compositions thereof may be stored stable at room temperature (e.g., at about 25° C.) for at least 1 month or longer.

In some embodiments, one or more assessments may be utilized during manufacture, or other preparation or use of polyribonucleotides (e.g., as a release test).

In some embodiments, one or more quality control parameters may be assessed to determine whether polyribonucleotides described herein meet or exceed acceptance criteria (e.g., for subsequent formulation and/or release for distribution). In some embodiments, such quality control parameters may include, but are not limited to RNA integrity, RNA concentration, residual DNA template and/or residual dsRNA. Certain methods for assessing RNA quality are known in the art; for example, one of skill in the art will recognize that in some embodiments, one or more analytical tests can be used for RNA quality assessment. Examples of such certain analytical tests may include but are not limited to gel electrophoresis, UV absorption, and/or PCR assay.

In some embodiments, a batch of polyribonucleotides may be assessed for one or more features as described herein to determine next action step(s). For example, a batch of polyribonucleotides can be designated for one or more further steps of manufacturing and/or formulation and/or distribution if RNA quality assessment indicates that such a batch of polyribonucleotides meet or exceed the relevant acceptance criteria. Otherwise, an alternative action can be taken (e.g., discarding the batch) if such a batch of polyribonucleotides does not meet or exceed the acceptance criteria.

In some embodiments, a batch of polyribonucleotides that satisfy assessment results can be utilized for one or more further steps of manufacturing and/or formulation and/or distribution.

### X. DNA Constructs

Among other things, the present disclosure provides DNA constructs, for example that may encode one or more antibody agents as described herein, or components thereof. In some embodiments, DNA constructs provided by and/or utilized in accordance with the present disclosure are comprised in a vector.

Non-limiting examples of a vector include plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). In some embodiments, a vector is an expression vector. In some embodiments, a vector is a cloning vector. In general, a vector is a nucleic acid construct that can receive or otherwise become linked to a nucleic acid element of interest (e.g., a construct that is or encodes a payload, or that imparts a particular functionality, etc.).

Expression vectors, which may be plasmid or viral or other vectors, typically include an expressible sequence of interest (e.g., a coding sequence) that is functionally linked with one or more control elements (e.g., promoters, enhancers, transcription terminators, etc.). Typically, such control elements are selected for expression in a system of interest. In some embodiments, a system is ex vivo (e.g., an in vitro transcription system); in some embodiments, a system is in vivo (e.g., a bacterial, yeast, plant, insect, fish, vertebrate, mammalian cell or tissue, etc.).

Cloning vectors are generally used to modify, engineer, and/or duplicate (e.g., by replication in vivo, for example in a simple system such as bacteria or yeast, or in vitro, such as by amplification such as polymerase chain reaction or other amplification process). In some embodiments, a cloning vector may lack expression signals.

In many embodiments, a vector may include replication elements such as primer binding site(s) and/or origin(s) of replication. In many embodiments, a vector may include insertion or modification sites such as restriction endonuclease recognition sites and/or guide RNA binding sites, etc.

In some embodiments, a vector is a viral vector (e.g., an AAV vector). In some embodiments, a vector is a non-viral vector. In some embodiments, a vector is a plasmid.

Those skilled in the art are aware of a variety of technologies useful for the production of recombinant polynucleotides (e.g., DNA or RNA) as described herein. For example, restriction digestion, reverse transcription, amplification (e.g., by polymerase chain reaction), Gibson assembly, etc., are well established and useful tools and technologies. Alternatively or additionally, certain nucleic acids may be prepared or assembled by chemical and/or enzymatic synthesis. In some embodiments, a combination of known methods is utilized to prepare a recombinant polynucleotide.

In some embodiments, polynucleotide(s) of the present disclosure are included in a DNA construct (e.g., a vector) amenable to transcription and/or translation.

In some embodiments, an expression vector comprises a polynucleotide that encodes proteins and/or polypeptides of the present disclosure operatively linked to a sequence or sequences that control expression (e.g., promoters, start signals, stop signals, polyadenylation signals, activators, repressors, etc.). In some embodiments, a sequence or sequences that control expression are selected to achieve a desired level of expression. In some embodiments, more than one sequence that controls expression (e.g., promoters) are utilized. In some embodiments, more than one sequence that controls expression (e.g., promoters) are utilized to achieve a desired level of expression of a plurality of polynucleotides that encode a plurality of proteins and/or polypeptides. In some embodiments, a plurality of recombinant proteins and/or polypeptides are expressed from the same vector (e.g., a bi-cistronic vector, a tri-cistronic vector, multi-cistronic). In some embodiments, a plurality of polypeptides are expressed, each of which is expressed from a separate vector.

In some embodiments, an expression vector comprising a polynucleotide of the present disclosure is used to produce a RNA and/or protein and/or polypeptide in a host cell. In some embodiments, a host cell may be in vitro (e.g., a cell line)—for example a cell or cell line (e.g., Human Embryonic Kidney (HEK cells), Chinese Hamster Ovary cells, etc.) suitable for producing polynucleotides of the present disclosure and proteins and/or polypeptides encoded by said polynucleotides.

In some embodiments, an expression vector is an RNA expression vector. In some embodiments, an RNA expression vector comprises a polynucleotide template used to produce a RNA in cell-free enzymatic mix. In some embodiments, an RNA expression vector comprising a polynucleotide template is enzymatically linearized prior to in vitro transcription. In some embodiments, a polynucleotide template is generated through PCR as a linear polynucleotide template. In some embodiments, a linearized polynucleotide is mixed with enzymes suitable for RNA synthesis, RNA capping and/or purification. In some embodiments, the resulting RNA is suitable for producing proteins encoded by the RNA.

A variety of methods are known in the art to introduce an expression vector into host cells. In some embodiments, a vector may be introduced into host cells using transfection. In some embodiments, transfection is completed, for example, using calcium phosphate transfection, lipofection, or polyethylenimine-mediated transfection. In some embodiments, a vector may be introduced into a host cell using transduction.

In some embodiments, transformed host cells are cultured following introduction of a vector into a host cell to allow for expression of said recombinant polynucleotides. In some embodiments, a transformed host cells are cultured for at least 12 hours, 16 hours, 20 hours, 24 hours, 28 hours, 32 hours, 36 hours, 40 hours, 44 hours, 48 hours, 52 hours, 56 hours, 60 hours, 64 hours, 68 hours, 72 hours or longer. Transformed host cells are cultured in growth conditions (e.g., temperature, carbon-dioxide levels, growth medium) in accordance with the requirements of a host cell selected. A skilled artisan would recognize culture conditions for host cells selected are well known in the art.

### EXEMPLARY NUMBERED EMBODIMENTS

Embodiment 1. A polyribonucleotide comprising a coding sequence that encodes a full-length Plasmodium CSP polypeptide, where the full-length Plasmodium CSP polypeptide comprises a Plasmodium CSP N-terminal domain, and where the coding sequence has an adenine content that is between 35% and 42%, and the portion of the coding sequence that encodes the Plasmodium CSP N-terminal domain has an adenine content that is at least 35%.

Embodiment 2. The polyribonucleotide of embodiment 1, where the portion of the coding sequence that encodes the Plasmodium CSP N-terminal domain has an adenine content that is between 35% and 45%.

Embodiment 3. The polyribonucleotide of embodiment 1 or 2, where the portion of the coding sequence that encodes the Plasmodium CSP N-terminal domain has an adenine content that is between 36% and 42%.

Embodiment 4. The polyribonucleotide of any one of embodiments 1-3, where the coding sequence has an adenine content that is between that is between 35% and 36.5%.

Embodiment 5. The polyribonucleotide of any one of embodiments 1-4, where the coding sequence has a uracil content that is between 14% and 20%, and the portion of the coding sequence that encodes the Plasmodium CSP N-terminal domain has a uracil content that is at least 10%.

Embodiment 6. The polyribonucleotide of any one of embodiments 1-5, where the coding sequence has a guanine content that is between 15% and 19.5%, and the portion of the coding sequence encoding a Plasmodium CSP N-terminal domain has a guanine content that is less than 27%.

Embodiment 7. The polyribonucleotide of any one of embodiments 1-6, where the coding sequence has a cytosine content that is between 22% and 31%, and the portion of the coding sequence that encodes the Plasmodium CSP N-terminal domain has a cytosine content that is less than 28%.

Embodiment 8. A polyribonucleotide comprising a coding sequence that encodes a full-length Plasmodium CSP polypeptide, where the full-length Plasmodium CSP polypeptide comprises a Plasmodium CSP N-terminal domain, and where the coding sequence has a uracil content that is between 14% and 20%, and the portion of the coding sequence that encodes the Plasmodium CSP N-terminal domain has a uracil content that is at least 10%.

Embodiment 9. The polyribonucleotide of embodiment 8, where the portion of the coding sequence that encodes the Plasmodium CSP N-terminal domain has a uracil content that is between 12% and 25%.

Embodiment 10. The polyribonucleotide of embodiment 8 or 9, where the portion of the coding sequence that encodes the Plasmodium CSP N-terminal domain has a uracil content that is between 17% and 22%.

Embodiment 11. The polyribonucleotide of any one of embodiments 8-10, where the coding sequence has a uracil content that is between that is between 15% and 16.5%.

Embodiment 12. The polyribonucleotide of any one of embodiments 8-11, where the coding sequence has an adenine content that is between 35% and 42%, and the portion of the coding sequence that encodes the Plasmodium CSP N-terminal domain has an adenine content that is at least 35%.

Embodiment 13. The polyribonucleotide of any one of embodiments 8-12, where the coding sequence has a guanine content that is between 15% and 19.5%, and the portion of the coding sequence encoding a Plasmodium CSP N-terminal domain has a guanine content that is less than 27%.

Embodiment 14. The polyribonucleotide of any one of embodiments 8-13, where the coding sequence has a cytosine content that is between 22% and 31%, and the portion of the coding sequence that encodes the Plasmodium CSP N-terminal domain has a cytosine content that is less than 28%.

Embodiment 15. A polyribonucleotide comprising a coding sequence that encodes a full-length Plasmodium CSP polypeptide, where the full-length Plasmodium CSP polypeptide comprises a Plasmodium CSP N-terminal domain, and where the coding sequence has a guanine content that is between 15% and 19.5%, and the portion of the coding sequence encoding a Plasmodium CSP N-terminal domain has a guanine content that is less than 27%.

Embodiment 16. The polyribonucleotide of embodiment 15, where the portion of the coding sequence that encodes the Plasmodium CSP N-terminal domain has a guanine content that is between 19% and 26%.

Embodiment 17. The polyribonucleotide of embodiment 15 or 16, where the portion of the coding sequence that encodes the Plasmodium CSP N-terminal domain has a guanine content that is between 19.5% and 24.5%.

Embodiment 18. The polyribonucleotide of any one of embodiments 15-17, where the coding sequence has a guanine content that is between that is between 18.5% and 19.5%.

Embodiment 19. The polyribonucleotide of any one of embodiments 15-18, where the coding sequence has an adenine content that is between 35% and 42%, and the portion of the coding sequence that encodes the Plasmodium CSP N-terminal domain has an adenine content that is at least 35%.

Embodiment 20. The polyribonucleotide of any one of embodiments 15-19, where the coding sequence has a uracil content that is between 14% and 20%, and the portion of the coding sequence that encodes the Plasmodium CSP N-terminal domain has a uracil content that is at least 10%.

Embodiment 21. The polyribonucleotide of any one of embodiments 15-20, where the coding sequence has a cytosine content that is between 22% and 31%, and the portion of the coding sequence that encodes the Plasmodium CSP N-terminal domain has a cytosine content that is less than 28%.

Embodiment 22. A polyribonucleotide comprising a coding sequence that encodes a full-length Plasmodium CSP polypeptide, where the full-length Plasmodium CSP polypeptide comprises a Plasmodium CSP N-terminal domain, and where the coding sequence has a cytosine content that is between 22% and 31%, and the portion of the coding sequence that encodes the Plasmodium CSP N-terminal domain has a cytosine content that is less than 28%.

Embodiment 23. The polyribonucleotide of embodiment 22, where the portion of the coding sequence that encodes the Plasmodium CSP N-terminal domain has a cytosine content that is between 12% and 27% cytosine.

Embodiment 24. The polyribonucleotide of embodiment 22 or 23, where the portion of the coding sequence that encodes the Plasmodium CSP N-terminal domain has a cytosine content that is between 15% and 22% cytosine.

Embodiment 25. The polyribonucleotide of any one of embodiments 22-24, where the coding sequence has a cytosine content that is between that is between 28.5% and 30% cytosine.

Embodiment 26. The polyribonucleotide of any one of embodiments 22-25, where the coding sequence has an adenine content that is between 35% and 42%, and the portion of the coding sequence that encodes the Plasmodium CSP N-terminal domain has an adenine content that is at least 35%.

Embodiment 27. The polyribonucleotide of any one of embodiments 22-26, where the coding sequence has a uracil content that is between 14% and 20%, and the portion of the coding sequence that encodes the Plasmodium CSP N-terminal domain has a uracil content that is at least 10%.

Embodiment 28. The polyribonucleotide of any one of embodiments 22-27, where the coding sequence has a guanine content that is between 15% and 19.5%, and the portion of the coding sequence encoding a Plasmodium CSP N-terminal domain has a guanine content that is less than 27%.

Embodiment 29. The polyribonucleotide of any one of embodiments 1-28, where the full-length Plasmodium CSP polypeptide comprises: (i) a secretory signal, (ii) the Plasmodium CSP N-terminal domain, where the Plasmodium CSP N-terminal domain comprises an N-terminal region, an N-terminal end region, and a junction region, (iii) a Plasmodium CSP central domain comprising a minor repeat region and a major repeat region, and (iv) a Plasmodium CSP C-terminal domain comprising a C-terminal region and a transmembrane region.

Embodiment 30. The polyribonucleotide of embodiment 29, where the secretory signal comprises or consists of a Plasmodium secretory signal.

Embodiment 31. The polyribonucleotide of embodiment 29 or 30, where the Plasmodium secretory signal comprises or consists of a Plasmodium CSP secretory signal.

Embodiment 32. The polyribonucleotide of embodiment 31, where the Plasmodium CSP secretory signal comprises or consists of an amino acid sequence according to SEQ ID NO: 4.

Embodiment 33. The polyribonucleotide of any one of embodiments 29-32, where (i) the N-terminal region comprises or consists of an amino acid sequence according to SEQ ID NO: 18; (ii) the N-terminal end region comprises or consists of an amino acid sequence according to SEQ ID NO: 19, and/or (iii) the junction region comprises or consists of an amino acid sequence according to SEQ ID NO: 20.

Embodiment 34. The polyribonucleotide of embodiment 33, where the Plasmodium CSP N-terminal domain comprises or consists of an amino acid sequence according to SEQ ID NO: 7.

Embodiment 35. The polyribonucleotide of any one of embodiments 29-34, where the minor repeat region comprises exactly three repeats of the amino acid sequence of NANPNVDP (SEQ ID NO: 29).

Embodiment 36. The polyribonucleotide of any one of embodiments 29-35, where the minor repeat region comprises or consists of an amino acid sequence according to SEQ ID NO: 26.

Embodiment 37. The polyribonucleotide of any one of embodiments 29-36, where the major repeat region comprises or consists of an amino acids sequence according to SEQ ID NO: 30.

Embodiment 38. The polyribonucleotide of any one of embodiments 29-37, where the Plasmodium CSP C-terminal domain comprises or consists of an amino acid sequence according to SEQ ID NO: 37.

Embodiment 39. The polyribonucleotide of any one of embodiments 1-38, where the full-length Plasmodium CSP polypeptide comprises an amino acid sequence of SEQ ID NO: 1.

Embodiment 40. The polyribonucleotide of any one of embodiments 1-39, where Plasmodium is Plasmodium falciparum.

Embodiment 41. The polyribonucleotide of embodiment 40, where Plasmodium falciparum is Plasmodium falciparum isolate 3D7.

Embodiment 42. The polyribonucleotide of any one of embodiments 29-41, where the portion of the coding sequence encoding the Plasmodium CSP secretory signal comprises or consists of a sequence according to SEQ ID NO: 6.

Embodiment 43. The polyribonucleotide of any one of embodiments 29-42, where the portion of the coding sequence encoding the Plasmodium CSP N-terminal domain comprises or consists of a sequence according to any one of SEQ ID NOs: 11, 13, 15, and 17.

Embodiment 44. The polyribonucleotide of any one of embodiments 29-43, where the portion of the coding sequence encoding the Plasmodium CSP N-terminal domain comprises or consists of a sequence according to SEQ ID NO: 13.

Embodiment 45. The polyribonucleotide of any one of embodiments 29-43, where the portion of the coding sequence encoding the Plasmodium CSP N-terminal domain comprises or consists of a sequence according to SEQ ID NO: 15.

Embodiment 46. The polyribonucleotide of any one of embodiments 29-45, where the portion of the coding sequence encoding the minor repeat region comprises or consists of a sequence according to SEQ ID NO: 28.

Embodiment 47. The polyribonucleotide of any one of embodiments 29-46, where the portion of the coding sequence encoding the major repeat region comprises or consists of a sequence according to SEQ ID NO: 32.

Embodiment 48. The polyribonucleotide of any one of embodiments 29-47, where the portion of the coding sequence encoding the central domain comprises or consists of a sequence according to SEQ ID NO: 25.

Embodiment 49. The polyribonucleotide of any one of embodiments 29-48, where the portion of the coding sequence encoding the C-terminal region comprises or consists of a sequence according to SEQ ID NO: 42.

Embodiment 50. The polyribonucleotide of any one of embodiments 29-49, where the portion of the coding sequence encoding the transmembrane region comprises or consists of a sequence according to SEQ ID NO: 45.

Embodiment 51. The polyribonucleotide of any one of embodiments 29-50, where the portion of the coding sequence encoding the Plasmodium CSP C-terminal domain comprises or consists of a sequence according to SEQ ID NO: 39.

Embodiment 52. The polyribonucleotide of any one of embodiments 1-51, where the coding sequence comprises or consists of a sequence according to any one of SEQ ID NOs: 50, 52, 54, 56, 58, 60, and 62.

Embodiment 53. The polyribonucleotide of any one of embodiments 1-52, where the coding sequence comprises or consists of a sequence according to SEQ ID NO: 52.

Embodiment 54. The polyribonucleotide of any one of embodiments 1-52, where the polyribonucleotide comprises or consists of a sequence according to SEQ ID NO: 54.

Embodiment 55. An RNA construct comprising in 5′ to 3′ order: (i) a 5′ UTR that comprises or consists of a modified human alpha-globin 5′-UTR; (ii) a polyribonucleotide of any one of embodiments 1-54; (iii) a 3′ UTR that comprises or consists of a first sequence from the amino terminal enhancer of split (AES) messenger RNA and a second sequence from the mitochondrial encoded 12S ribosomal RNA; and (iv) a polyA tail sequence.

Embodiment 56. The RNA construct of embodiment 55, where the 5′ UTR comprises or consists of a ribonucleic acid sequence according to SEQ ID NO: 63.

Embodiment 57. The RNA construct of embodiment 55 or 56, where the 3′ UTR comprises or consists of a ribonucleic acid sequence according to SEQ ID NO: 65.

Embodiment 58. The RNA construct of any one of embodiments 55-57, where the polyA tail sequence is a split polyA tail sequence.

Embodiment 59. The RNA construct of embodiment 58, where the split polyA tail sequence comprises or consists of a ribonucleic acid sequence according to SEQ ID NO: 66.

Embodiment 60. The RNA construct of any one of embodiments 55-59, further comprises a 5′ cap.

Embodiment 61. The RNA construct of any one of embodiments 55-60, further comprises a cap proximal sequence comprising positions +1, +2, +3, +4, and +5 of the polyribonucleotide.

Embodiment 62. The RNA construct of embodiment 60 or 61, where the 5′ cap comprises or consists of m7(3′OMeG)(5′)ppp(5′)(2′OMeA₁)pG₂, where A₁ is position +1 of the polyribonucleotide, and G₂ is position +2 of the polyribonucleotide.

Embodiment 63. The RNA construct of embodiment 61 or 62, where the cap proximal sequence comprises A₁ and G₂ of the Cap1 structure, and a sequence comprising: A₃A₄U₅ at positions +3, +4 and +5 respectively of the polyribonucleotide.

Embodiment 64. The RNA construct of any one of embodiments 55-63, where the RNA construct comprises a sequence of any one of SEQ ID NOs: 70 to 76.

Embodiment 65. The RNA construct of any one of embodiments 55-64, where the RNA construct comprises a sequence of SEQ ID NO: 71.

Embodiment 66. The RNA construct of any one of embodiments 55-63, where the RNA construct comprises a sequence of SEQ ID NO: 72.

Embodiment 67. A composition comprising one or more polyribonucleotides of any one of embodiments 1-54.

Embodiment 68. A composition comprising one or more RNA constructs of any one of embodiments 55-66.

Embodiment 69. The composition of embodiment 67 or 68, where the composition further comprises lipid nanoparticles, polyplexes (PLX), lipidated polyplexes (LPLX), or liposomes, where the one or more polyribonucleotides are fully or partially encapsulated within the lipid nanoparticles, polyplexes (PLX), lipidated polyplexes (LPLX), or liposomes.

Embodiment 70. The composition of any one of embodiments 67-69, where the composition further comprises lipid nanoparticles, where the one or more polyribonucleotides are encapsulated within the lipid nanoparticles.

Embodiment 71. The composition of embodiment 69 or 70, where the lipid nanoparticles target liver cells.

Embodiment 72. The composition of embodiment 69 or 70, where the lipid nanoparticles target secondary lymphoid organ cells.

Embodiment 73. The composition of embodiment any one of embodiments 69-72, where the lipid nanoparticles are cationic lipid nanoparticles.

Embodiment 74. The composition of any one of embodiments 69-73, where the lipid nanoparticles each comprise: (a) a polymer-conjugated lipid; (b) a cationic lipid; and (c) one or more neutral lipids.

Embodiment 75. The composition of embodiment 74, where the polymer-conjugated lipid comprises a PEG-conjugated lipid.

Embodiment 76. The composition of embodiment 74 or 75, where the polymer-conjugated lipid comprises 2-[(polyethylene glycol)-2000]-N,N-ditetradecylacetamide.

Embodiment 77. The composition of any one of embodiments 74-76, where the one or more neutral lipids comprise 1,2-Distearoyl-sn-glycero-3-phosphocholine (DPSC).

Embodiment 78. The composition of any one of embodiments 74-77, where the one or more neutral lipids comprise cholesterol.

Embodiment 79. The composition of any one of embodiments 74-78, where the cationic lipid comprises [(4-Hydroxybutyl)azanediyl]di(hexane-6,1-diyl) bis(2-hexyldecanoate).

Embodiment 80. The composition of any one of embodiments 74-79, where the lipid nanoparticles have an average diameter of about 50-150 nm.

Embodiment 81. A pharmaceutical composition comprising the composition of any one of embodiments 67-80 and at least one pharmaceutically acceptable excipient.

Embodiment 82. The pharmaceutical composition of embodiment 81, where the pharmaceutical comprises a cryoprotectant, optionally where the cryoprotectant is sucrose.

Embodiment 83. The pharmaceutical composition of embodiment 81 or 82, where the pharmaceutical comprises an aqueous buffered solution, optionally where the aqueous buffered solution comprises one or more of Tris base, Tris HCl, NaCl, KCl, Na₂HPO₄, and KH₂PO₄.

Embodiment 84. A method comprising administering a polyribonucleotide according to any one of embodiments 1-54 to a subject.

Embodiment 85. A method comprising administering an RNA construct according to any one of embodiments 55-66 to a subject.

Embodiment 86. A method comprising administering a composition according to any one of embodiments 67-80 to a subject.

Embodiment 87. A method comprising administering one or more doses of the pharmaceutical composition of any one of embodiments 81-83 to a subject.

Embodiment 88. The pharmaceutical composition of any one of embodiments 81-83 for use in the treatment of a malaria infection comprising administering one or more doses of the pharmaceutical composition to a subject.

Embodiment 89. The pharmaceutical composition of any one of embodiments 81-83 for use in the prevention of a malaria infection comprising administering one or more doses of the pharmaceutical composition to a subject.

Embodiment 90. The method of embodiment 87 or the pharmaceutical composition for use of embodiment 88 or 89, comprising administering two or more doses of the pharmaceutical composition to a subject.

Embodiment 91. The method of embodiment 87 or 90, or the pharmaceutical composition for use of any one of embodiments 88-90, comprising administering three or more doses of the pharmaceutical composition to a subject.

Embodiment 92. The method or the pharmaceutical composition for use of embodiment 91, where the second of the three or more doses is administered to the subject at least 4 weeks after the first of the three or more doses is administered to the subject.

Embodiment 93. The method or the pharmaceutical composition for use of embodiment 91 or 92, where the third of the three or more doses is administered to the subject at least 4 weeks after the second of the three or more doses is administered to the subject.

Embodiment 94. The method of any one of embodiments 90-93, or the pharmaceutical composition for use of any one of embodiments 88-93, comprising administering a fourth dose of the pharmaceutical composition to a subject.

Embodiment 95. The method or the pharmaceutical composition for use of embodiment 94, where the fourth dose is administered to the subject at least one year after the third of the three or more doses is administered to the subject.

Embodiment 96. A combination comprising: (i) a first pharmaceutical composition comprising a first polyribonucleotide, where the first polyribonucleotide is a polyribonucleotide according to any one of embodiments 1-54; and (ii) a second pharmaceutical composition comprising a second polyribonucleotide, where the second polyribonucleotide encodes a second polypeptide, and the second polypeptide comprises one or more Plasmodium T-cell antigens.

Embodiment 97. A method comprising administering a combination of embodiment 96 to a subject.

Embodiment 98. The method of embodiment 97, where the first pharmaceutical composition and the second pharmaceutical composition are administered on the same day.

Embodiment 99. The method of embodiment 97, where the first pharmaceutical composition and the second pharmaceutical composition are administered on different days.

Embodiment 100. The method of any one of embodiments 96-99, where the first pharmaceutical composition and the second pharmaceutical composition are administered to the subject at different locations on the subject's body.

Embodiment 101. The method of any one of embodiments 96-100, where the method is a method of treating a malaria infection.

Embodiment 102. The method of any one of embodiments 96-100, where the method is a method of preventing a malaria infection.

Embodiment 103. The method of any one of embodiments 96-102, where the subject has or is at risk of developing a malaria infection.

Embodiment 104. The method of any one of embodiments 96-103, where the subject is a human.

Embodiment 105. The method of any one of embodiments 96-104, where administration induces an anti-malaria immune response in the subject.

Embodiment 106. The method of embodiment 105, where the anti-malaria immune response in the subject comprises an adaptive immune response.

Embodiment 107. The method of embodiment 105 or 106, where the anti-malaria immune response in the subject comprises a T-cell response.

Embodiment 108. The method of embodiment 107, where the T-cell response is or comprises a CD4+ T cell response.

Embodiment 109. The method of embodiment 107 or 108, where the T-cell response is or comprises a CD8+ T cell response.

Embodiment 110. The method of any one of embodiments 105-109, where the anti-malaria immune system response comprises a B-cell response.

Embodiment 111. The method of any one of embodiments 105-110, where the anti-malaria immune system response comprises the production of antibodies directed against CSP.

Embodiment 112. Use of the pharmaceutical composition of any one of embodiments 81-83 in the treatment of a malaria infection.

Embodiment 113. Use of the pharmaceutical composition of any one of embodiments 81-83 in the prevention of a malaria infection.

Embodiment 114. Use of the pharmaceutical composition of any one of embodiments 81-83 in inducing an anti-malaria immune response in a subject.

Embodiment 115. A host cell comprising a polyribonucleotide of any one of embodiments 1-54.

Embodiment 116. A host cell comprising an RNA construct of any one of embodiments 55-66.

The disclosure is further illustrated by the following examples. The examples are provided for illustrative purposes only. They are not to be construed as limiting the scope or content of the disclosure in any way.

### EXEMPLIFICATION

### Example 1: a Codon Optimized Polyribonucleotide Encoding Full-Length CSP Exhibits an Irregular Peak

This example describes synthesis and characterization of a polyribonucleotide encoding full-length PfCSP construct. Specifically, the present example describes in vitro transcription of a particular exemplary construct, RNA construct 23, and characterization of RNA integrity by capillary electrophoresis. The present example identifies a problem with this construct, in that it is exhibits an irregular peak when analyzed by capillary electrophoresis.

CleanCap (m7(3′OMeG)(5′)ppp(5′)(2′OMeA)pG) capped RNA corresponding to RNA construct 23 was produced following by in vitro transcription, under the following conditions: 3 mM CleanCap, 12.2 mM ATP/CTP, G/m1Y fed batch, 180 min, at pH 7.0 and pH 8.35. RNA construct 23 has a sequence of:

(SEQ ID NO: 69)
AGAAUAAACUAGUAUUCUUCUGGUCCCCACAGACUCAGAGAGAACC
CGCCACCAUGAUGCGGAAGCUGGCCAUCCUGAGCGUGUCCAGCUU
CCUGUUUGUGGAAGCCCUGUUCCAAGAGUACCAGUGCUACGGCAG
CAGCAGCAACACCAGAGUGCUGAACGAGCUGAACUACGACAACGC
CGGCACCAACCUGUACAACGAGCUGGAAAUGAACUACUACGGCAA
GCAAGAGAACUGGUACAGCCUGAAGAAGAACAGCAGAAGCCUGGG
CGAGAACGACGACGGCAACAACGAGGACAACGAGAAGCUGCGGAA
GCCCAAGCACAAGAAGCUGAAGCAGCCCGCCGACGGCAAUCCCGA
UCCUAACGCCAAUCCUAACGUGGACCCCAACGCUAACCCCAAUGU
GGACCCAAAUGCCAAUCCAAAUGUCGAUCCCAAUGCAAACCCUAA
CGCAAACCCGAAUGCUAACCCAAACGCUAAUCCGAACGCAAAUCC
CAAUGCCAAUCCUAAUGCUAAUCCCAACGCCAAUCCAAACGCUAA
CCCUAAUGCAAAUCCUAACGCUAACCCCAACGCAAACCCGAACGC
CAAUCCUAACGCCAAUCCGAAUGCAAACCCCAAUGCUAACCCCAA
UGCAAAUCCCAAUGUGGACCCUAACGCAAAUCCUAAUGCAAAUCC
GAACGCAAAUCCUAACGCAAACCCAAACGCCAAUCCAAAUGCAAA
CCCUAACGCUAAUCCAAAUGCUAAUCCCAAUGCAAACCCGAAUGC
AAAUCCCAACGCUAACCCUAACGCUAACCCCAAUGCCAAUCCAAA
CGCAAAUCCAAAUGCUAACCCGAACGCUAACCCAAACGCAAACCC
AAAUGCUAAUCCUAACAAGAACAAUCAAGGCAACGGCCAGGGCCA
CAACAUGCCCAACGAUCCCAACAGAAACGUGGACGAGAACGCCAA
CGCCAACAGCGCCGUGAAGAACAACAACAAUGAGGAACCCAGCGA
CAAGCACAUCAAAGAGUACCUGAACAAGAUCCAGAACAGCCUGAG
CACCGAGUGGUCCCCAUGUAGCGUGACAUGCGGCAACGGAAUCCA
AGUGCGGAUCAAGCCUGGCAGCGCCAACAAGCCUAAGGAUGAGCU
GGACUACGCCAAUGACAUCGAGAAGAAAAUCUGCAAGAUGGAAAA
GUGCAGCAGCGUGUUCAACGUGGUCAACAGCAGCAUCGGCCUGAU
UAUGGUGCUGUCCUUCCUGUUCCUGAACUGAUGACUGGUACUGCA
UGCACGCAAUGCUAGCUGCCCCUUUCCCGUCCUGGGUACCCCGAG
UCUCCCCCGACCUCGGGUCCCAGGUAUGCUCCCACCUCCACCUGC
CCCACUCACCACCUCUGCUAGUUCCAGACACCUCCCAAGCACGCA
GCAAUGCAGCUCAAAACGCUUAGCCUAGCCACACCCCCACGGGAA
ACAGCAGUGAUUAACCUUUAGCAAUAAACGAAAGUUUAACUAAGC
UAUACUAACCCCAGGGUUGGUCAAUUUCGUGCCAGCCACACCAAA
AAAAAAAAAAAAAAAAAAAAAAAAAAAGCAUAUGACUAAAAAAAA
AAAAAAAAAAAAAAAAAAAAAAAAAAAAAAAAAAAAAAAAAAAAA
AAAAAAAAAAAAAAAAA.

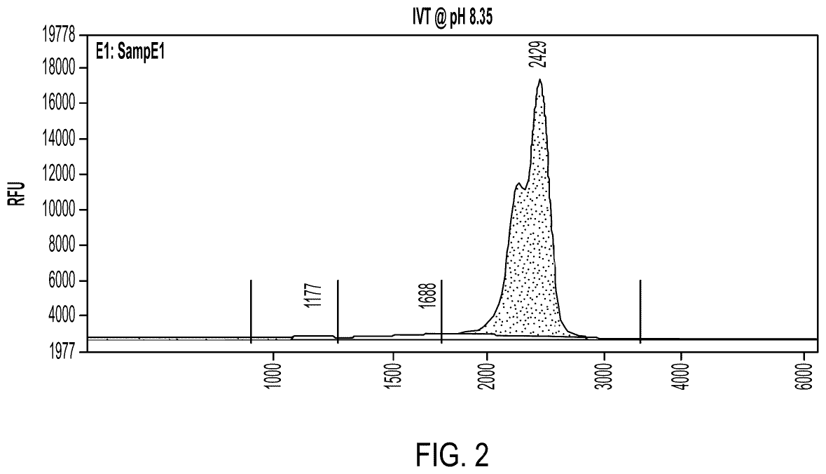

RNA integrity was assessed by microfluidic-based electrophoresis, specifically using a Fragment Analyzer (FA; Agilent Technologies), Standard Sensitivity Kit (Agilent Technologies, article no. DNF-471-0500) and ProSize software (Agilent Technologies). Results are shown in FIG. 1 and FIG. 2. RNA construct 23 was observed to have an irregular peak having a shoulder or a double peak when in vitro transcribed at pH 7 and pH 8.35, respectively.

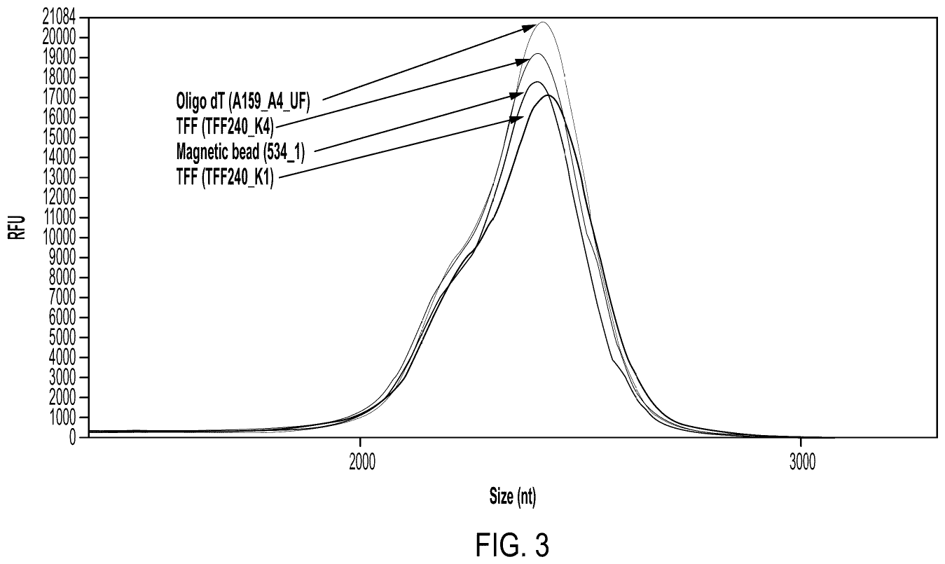

In vitro transcription at pH 7.0 was performed and resulting RNA was purified using different methods: Tangential Flow Filtration (TFF), magnetic bead purification, or oligo-dT affinity chromatography. RNA integrity was assessed using a Fragment Analyzer (FA; Agilent Technologies). Results are shown in FIG. 3. No significant difference in the peak shape was observed when the RNA was purified using these different methods. These results demonstrate the presence of an irregular peak and confirm that the irregular peak is independent of the means of RNA purification.

Moreover, this irregular peak shape was consistently observed from different batches and when purified on different days. Three different batches of RNA construct 23 were analyzed in triplicate on two different days. There was high reproducibility between the batches and different measurements, as assessed by total integrity, however, variability was observed when evaluating the main peak and shoulder separately (data not shown).

Accordingly, the present example identifies an irregular peak issue with a particular RNA construct encoding a full-length CSP having an RNA sequence of SEQ ID NO: 48. This irregular peak issue is consistent between batches and when purified by different purification methods.

### Example 2: No Evidence of an Impurity with the Irregular Peak of RNA Construct 23

The present disclosure encompasses a recognition that an irregular peak is inconsistent with GMP practices, which require a single peak to ensure RNA integrity. This example describes a series of characterizations of the irregular peak observed with an exemplary full-length PfCSP construct, RNA construct 23. Specifically, this example describes several different analyses that were conducted to assess whether the irregular peak is the result of an impurity or degradation product.

### Forced Degradation Analysis

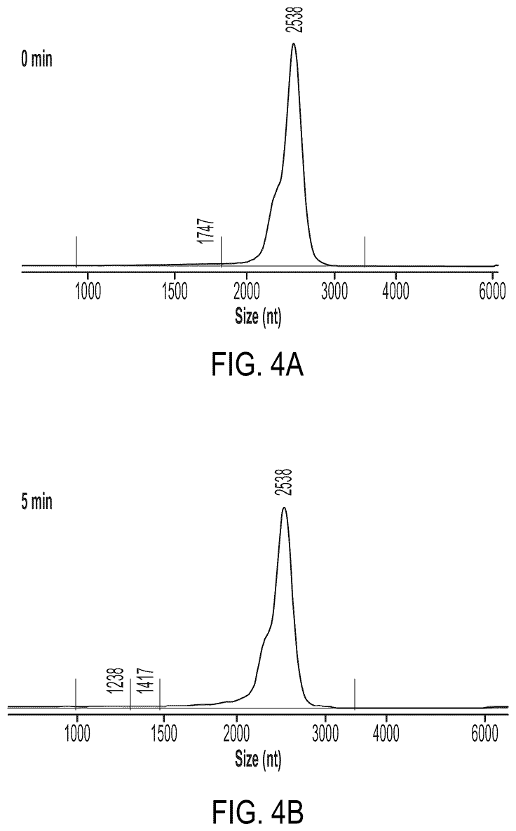

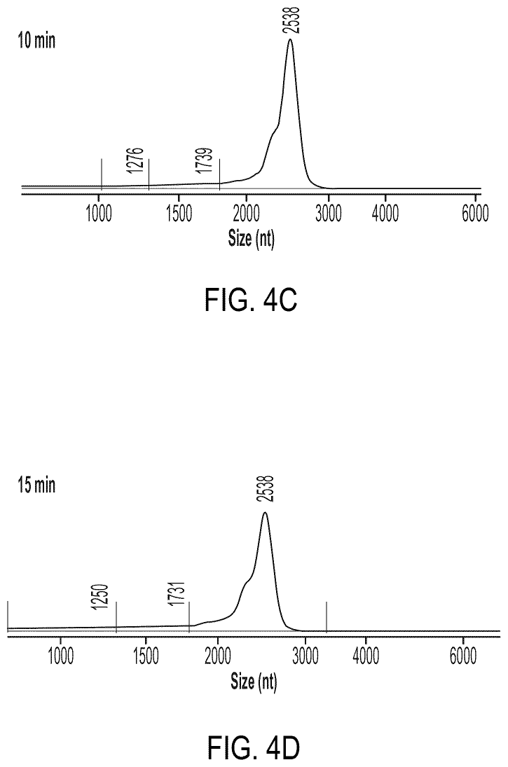

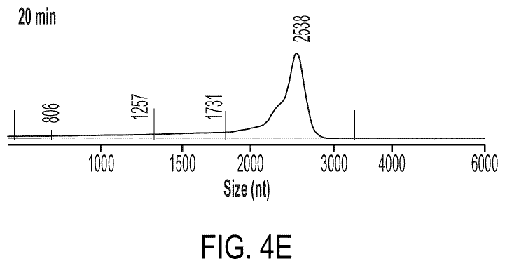

A forced degradation study at 90° C. was performed and degradation analyzed by a Fragment Analyzer (FA, Agilent Technologies) and PA 800 Plus (Sciex), both of which are capable of detecting degradation products. Exemplary fragment analyzer results are depicted in FIGS. 4A-4E. It was found that the shoulder of the peak degrades in a similar way as main peak, whether detected by FA (FIGS. 4A-4E) or PA 800 Plus (data not shown).

### Western Blot Analysis

An exemplary full-length CSP construct, RNA construct 23, was purified by TFF or TFF+magnetic beads and protein expression after transfection into cells was analyzed by western blot. Specifically, HEK293T cells were transfected with 250 ng of RNA construct 23, using RiboJuice™ as a transfection reagent. Eighteen hours post transfection, cells were harvested, lysed with a lysis buffer, and processed for western blot analysis in a 4-15% gel. For both methods of RNA purification, a single band was detected and this band was observed at the expected size of about 42 kDa (data not shown). This size is consistent with published data, see Lopaticki et al. (2017) Nat. Commun. 8(1):561. This analysis shows that despite the irregular shaped peak, and independent of the purification method, only a single translation product is detected by western blot.

### Translatability Analysis

An exemplary full-length CSP construct, RNA construct 23, was also analyzed by a translatability assay. RNA construct 23 was purified using magnetic beads and in vitro translated with biotinylated lysine. Biotinylated lysine is incorporated in the protein, allowing for subsequent visualization. A single translation product was detected, having the expected size (data not shown).

### HPLC Fractionation Analysis

An exemplary full-length CSP construct, RNA construct 23, was further analyzed by HPLC fractionation. Specifically, ion-pair reversed phase (IP-RP) chromatography coupled to UV detection was used to separate RNA by size and hydrophobicity. HPLC fractionation was performed in TEA/HFIP buffer, at 75° C.; SpeedVac @40° C. IP-RP fractions corresponding to the shoulder (Fraction 1), the first half of the main peak (Fraction 2), and the remainder of the main peak (Fraction 3) were collected.

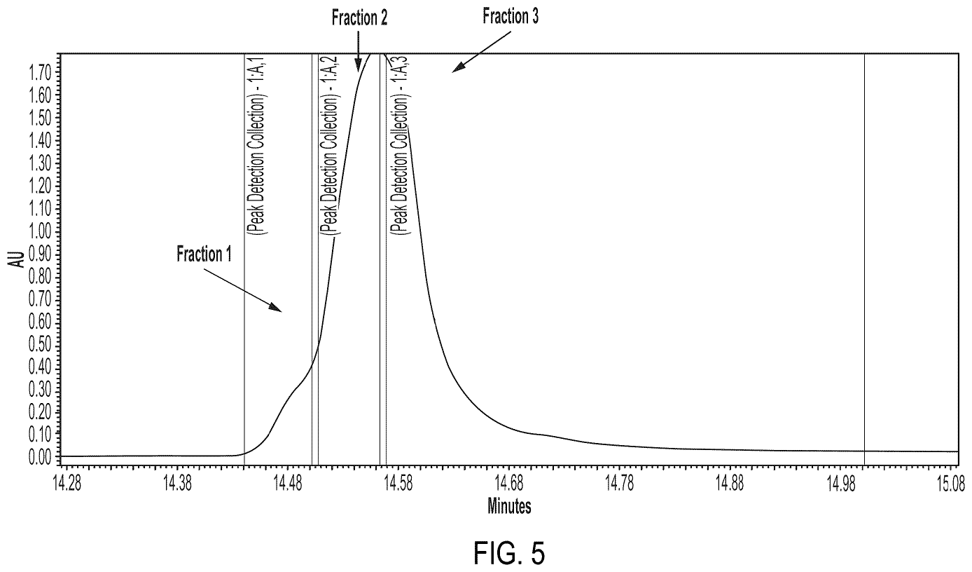

Each of these fractions were then separately analyzed by repeat HPLC analysis, and results are shown in FIG. 5. Re-injected fractioned RNA samples each exhibited the original irregular peak shape upon subsequent HPLC analysis. The pooled fractions and the unfractioned RNA also exhibited this same irregular peak.

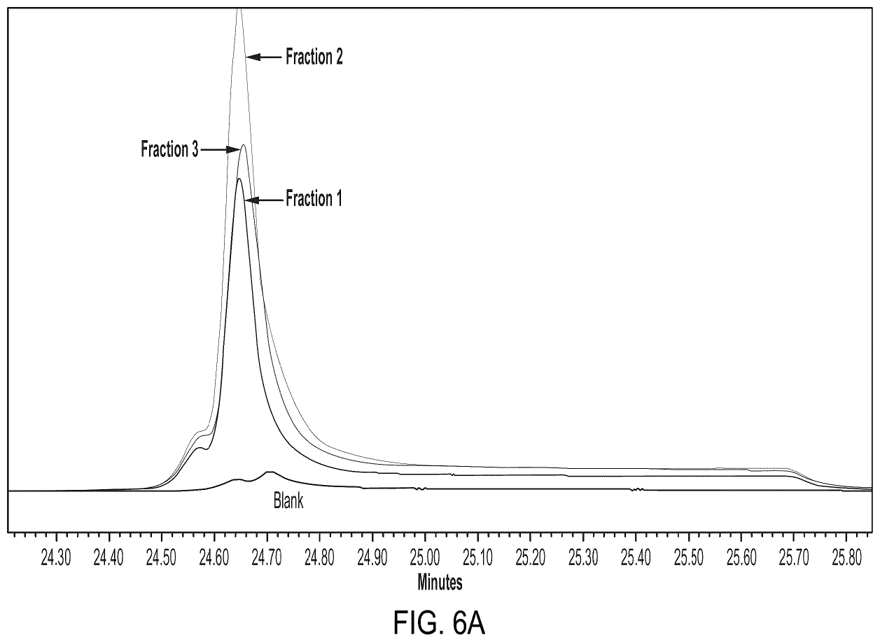

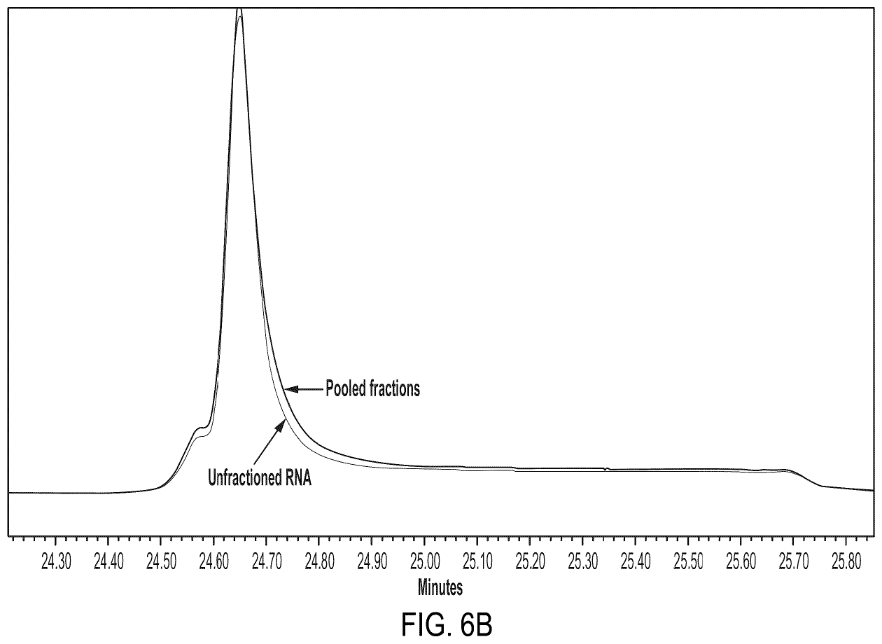

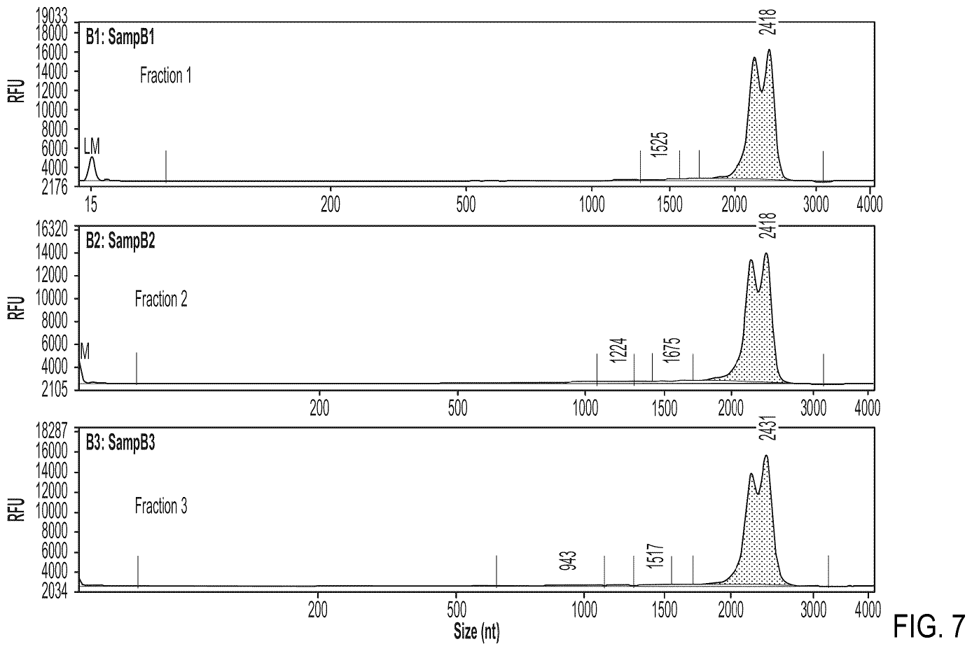

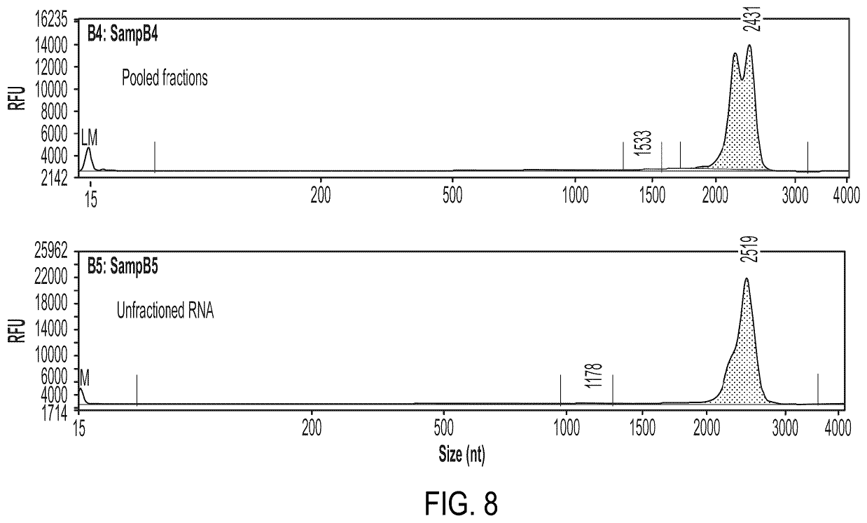

The separate fractions were also analyzed by capillary electrophoresis using a Fragment Analyzer (FA; Agilent Technologies) and results are shown in FIGS. 6A-6B. Each of the fractions exhibited an irregular double peak; the different fractions were not distinguishable by HPLC/FA analysis.

These results show that it was not possible to separate the double peak/shoulder of the irregular peak by HPLC. Notably, HPLC fractionation analysis has previously been used to identify contaminating species for other, unrelated RNA constructs. For example, HPLC fractionation analysis of irregular peaks have previously identified shorter RNA fragments that lack a poly(A) tail and/or a 5′ Cap. Accordingly, the results of the present HPLC analysis of RNA construct 23 are consistent with the irregular peak corresponding to a single species.

### Sanger Sequencing

The polynucleotide identity of the irregular peak was analyzed by reverse transcription and DNA sequencing. RNA construct 23 was analyzed by denaturing agarose gel electrophoresis and only a single band was visible. RNA was then converted to cDNA by reverse transcriptions, which was performed at 55° C. and 65° C. using SuperScript IV, and the cDNA amplified by PCR using primers in the 5′-UTR and 3′-UTR, 29 cycles, followed by gel electrophoresis. Sanger sequencing was performed on cDNA synthesized at 65° C. Sanger sequencing confirmed identity of coding sequence and found no indication for second species.

### Nanopore Sequencing

The polynucleotide identity of the irregular peak was analyzed by Nanopore. RNA construct 23 was analyzed by direct RNA sequencing. The sample was converted into a library by attaching a 3′-adapter to the RNA and sequenced with the help of a Nanopore flow cell (Oxford Nanopore Technologies Limited), followed by basecalling translating the electric signals into a nucleotide sequence. Nanopore sequencing confirmed identity and found no indication for second species.

Taken together, these characterization studies all support that the irregular peak corresponds to a single species. Without wishing to be bound by theory, it is hypothesized that the irregular peak associated with RNA construct 23 may be the result of different secondary structures of a single RNA sequence.

### Example 3: Optimizing the Nucleotide Content of the CSP N-Terminal Domain Resolves the Irregular Peak

The present disclosure provides the unexpected discovery that selectively optimizing the nucleotide content of the N-terminal domain region of an exemplary full-length PfCSP construct, RNA construct 23, resolves the irregular peak associated with this construct. Specifically, this example describes several RNA construct variants where the nucleotide content of the portion that encodes the Plasmodium CSP N-terminal domain has been optimized. These variant constructs were found to have an improved peak when analyzed by a Fragment Analyzer and/or Bioanalyzer.

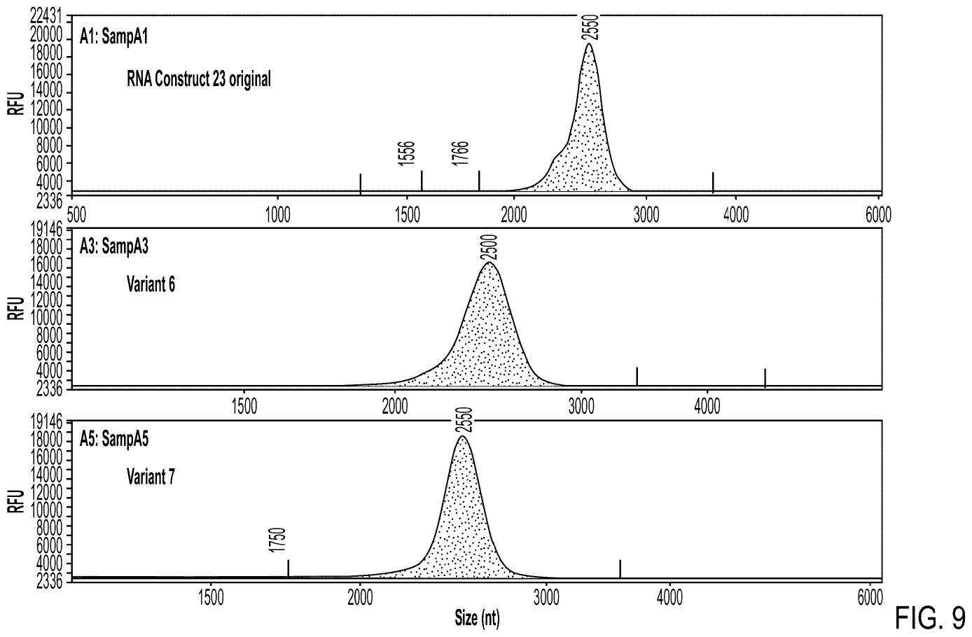

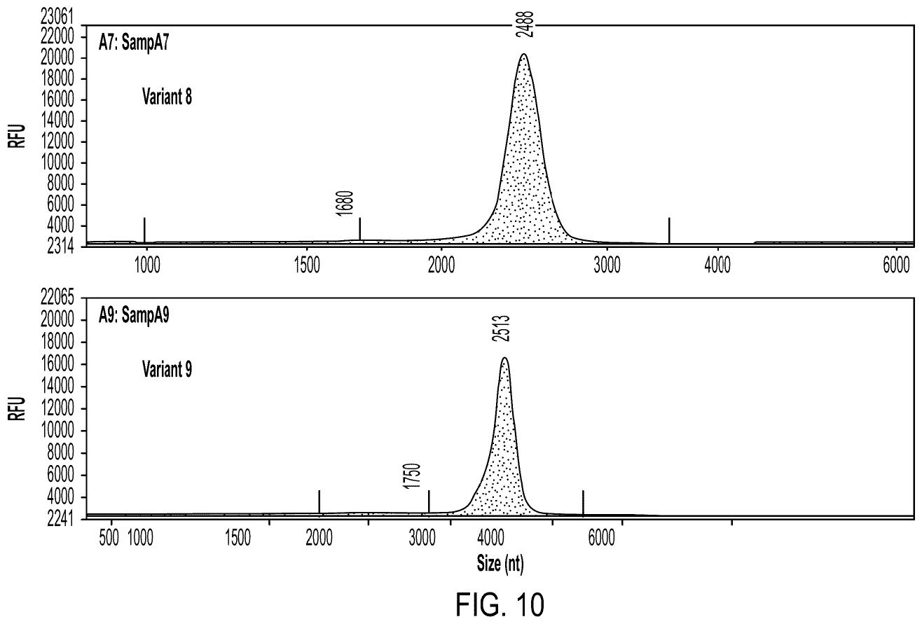

Nucleotide variants of RNA construct 23 were made, which encode the same polypeptide as RNA construct 23, namely, a polypeptide comprising amino acids 1-397 of SEQ ID NO:1. It was observed that those variants that specifically optimize the nucleotide content of the portion encoding the N-terminal region of CSP had an improved peak when analyzed by a Fragment Analyzer. Specifically, variants 6, 7, 8, 9, 10, 13, and 14 all showed an improved peak shape. Notably, variant 7 and variant 8 consistently exhibited single peak. See FIGS. 9 and 10 and Table 5 below.

TABLE 5
Nucleotide content of N terminal region
of RNA Construct 23 variants

| | N term | N term | N term | N term | Fragment |
|---|---|---|---|---|---|
| Construct | % A | % U | % G | % C | Analyzer Peak |
| RNA | 34.51% | 9.41% | 27.45% | 28.63% | Double peak |
| construct 23 | | | | | |
| Variant 4 | 34.51% | 9.41% | 27.45% | 28.63% | Double peak |
| Variant 5 | 34.51% | 9.41% | 27.45% | 28.63% | Double peak |
| Variant 6 | 44.71% | 23.92% | 19.22% | 12.16% | Single peak with |
| | | | | | fronting |
| Variant 7 | 41.96% | 21.96% | 20.00% | 16.08% | Single peak |
| Variant 8 | 36.86% | 17.25% | 24.31% | 21.57% | Single peak |
| Variant 9 | 35.69% | 16.86% | 24.71% | 22.75% | Single peak with |
| | | | | | fronting |
| Variant 10 | 41.96% | 21.96% | 20.00% | 16.08% | Single peak with |
| | | | | | fronting |
| Variant 12 | 34.51% | 9.41% | 27.45% | 28.63% | Double peak |
| Variant 13 | 41.96% | 21.96% | 20.00% | 16.08% | Variable |
| Variant 14 | 41.96% | 21.96% | 20.00% | 16.08% | Single peak, but |
| | | | | | degraded |

TABLE 6
Nucleotide content of RNA Construct 23 variants

| | Full | Full | Full | Full | | | | |
|---|---|---|---|---|---|---|---|---|
| | coding | coding | coding | coding | Full | Full | Full | Full |
| | region | region | region | region | construct | construct | construct | construct |
| Construct | % A | % U | % G | % C | % A | % U | % G | % C |
| RNA construct 23 | 34.75% | 13.62% | 20.13% | 31.50% | 36.81% | 14.10% | 18.50% | 30.59% |
| Variant 4 | 37.01% | 17.04% | 17.88% | 28.07% | 38.46% | 16.61% | 16.85% | 28.08% |
| Variant 5 | 36.68% | 16.46% | 18.46% | 28.40% | 38.22% | 16.18% | 17.28% | 28.33% |
| Variant 6 | 36.93% | 16.71% | 18.38% | 27.99% | 38.40% | 16.36% | 17.22% | 28.02% |
| Variant 7 | 36.34% | 16.29% | 18.55% | 28.82% | 37.97% | 16.06% | 17.34% | 28.63% |
| Variant 8 | 35.25% | 15.29% | 19.47% | 29.99% | 37.18% | 15.32% | 18.01% | 29.49% |
| Variant 9 | 35.00% | 15.20% | 19.55% | 30.24% | 37.00% | 15.26% | 18.07% | 29.67% |
| Variant 10 | 38.26% | 19.13% | 16.88% | 25.73% | 39.38% | 18.13% | 16.12% | 26.37% |
| Variant 12 | 34.67% | 13.95% | 19.88% | 31.50% | 36.75% | 14.35% | 18.32% | 30.59% |
| Variant 13 | 36.26% | 16.62% | 18.30% | 28.82% | 37.91% | 16.30% | 17.16% | 28.63% |
| Variant 14 | 42.86% | 20.80% | 16.04% | 20.30% | 42.74% | 19.35% | 15.51% | 22.41% |

Notably, all constructs tested that retained the oligonucleotide content of the N-terminal region (34.51% A, 9.41% U, 27.45% G, 28.63% C) exhibited a double peak in RNA integrity analysis using either a Fragment Analyzer or a Bioanalyzer. See, e.g., Table 6 above.

Accordingly, this data supports that the nucleotide content of the portion of CSP encoding the N-terminal region specifically has an effect on the RNA integrity analysis. Without wishing to be bound by theory, it is suggested that optimizing the N terminal region will affect the secondary structure of the RNA construct.

### Example 4: Expression of Full-Length CSP Construct Optimized Variants

The present Example demonstrates that exemplary optimized polyribonucleotides as described herein, exhibit in-vitro expression (e.g., intracellular, surface) in mammalian cells (HEK293T cells).

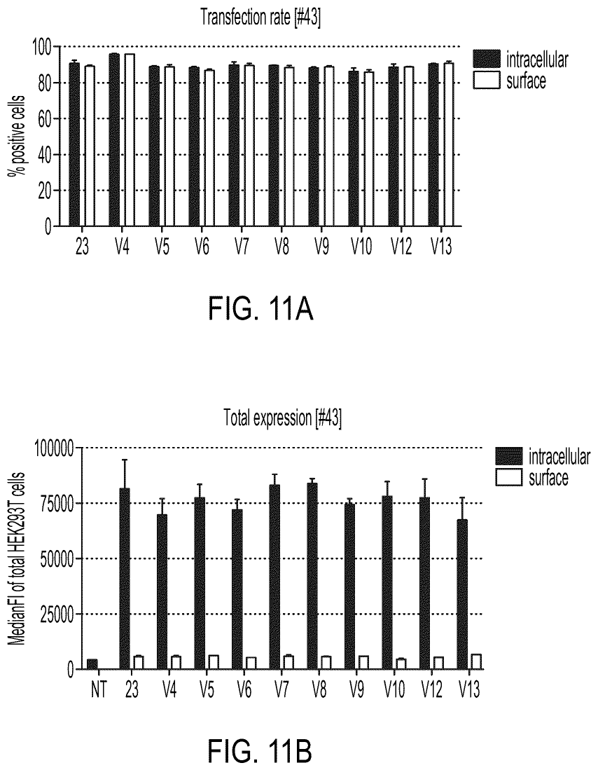

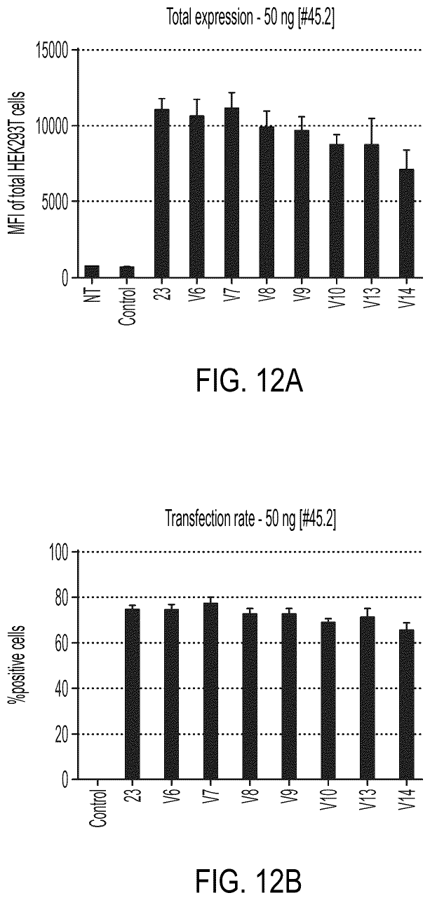

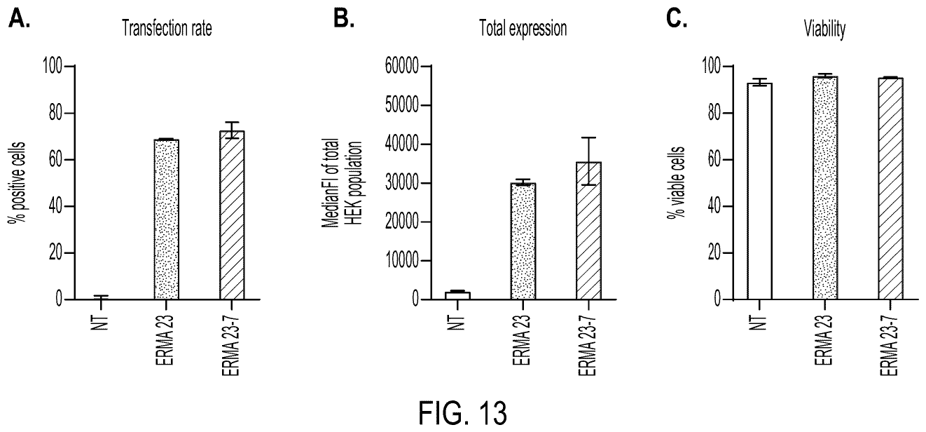

Briefly, HEK293T cells were transfected with (i) 200 ng of RNA constructs or (ii) 50 ng of RNA constructs with MessengerMax as transfection reagent (similar to LNPs as it forms particles). HEK293T cells transfected with 12.5 ng (see FIG. 13), 100 ng (see FIG. 12), and 200 ng (see FIG. 11) RNA constructs and assessed for protein expression by antibody staining and flow cytometry. Transfection rate was determined by measuring percentage of positive cells, and total expression was determined by measuring median fluorescence of the total HEK population.

Results are shown in FIG. 11 and FIG. 12. RNA constructs had an overall high transfection rate. Variants 6 to 9 all show similar or higher expression than RNA Construct 23, particularly when looking at total expression, which measures expression in the overall live HEK population.

For the direct comparison of RNA Construct 23 and variant 7, HEK293T cells were transfected with 12.5 ng of either RNA formulated with the transfection reagent MessengerMax. 18 hours (h) later, they were harvested and stained with a viability dye and, after permeabilization, with an anti-PfCSP monoclonal antibody. Flow cytometry was used to quantify the percentage of transfected cells (designated transfection rate), total protein expression and cell viability. Results are shown in FIG. 13.

### Example 5: In Vivo Immunogenicity of Exemplary Polyribonucleotides Encoding Full-Length Plasmodium CSP Polypeptide Constructs

The present Example demonstrates that exemplary polyribonucleotides encoding full-length Plasmodium CSP polypeptide constructs, as described herein, are immunogenic in vivo. In particular, RNA Construct 23 and variant 7 were evaluated in vivo in immunogenicity studies in mice.

C57BL6 female mice (10-12-weeks old) were immunized intramuscularly (IM) twice, for example, on days 0 and 21, with 1 μg of a formulated RNA construct 23 or variant 7 (described in Example 4) or injected with phosphate buffer saline (vehicle) (n=8 mice/group). Blood samples were collected pre-immunization (day 0) and after the first dose (e.g., on days 14, 21, 28 and 35) to generate serum samples at various time points. At the end of the experiment (e.g., day 35), splenocytes were harvested and analyzed by fluorospot.

Serum samples obtained from each group of immunized animals were analyzed by one or more of the following method(s): (1) Enzyme-linked Immunosorbent Assay (ELISA), (2) multiplex assay, (3) sporozoite ELISA, (4) traversal Assay, (5) inhibition of Liver Stage Development Assay (ILSDA), (6) Surface Plasmon Resonance (SPR), and/or (7) fluorospot assay.

### (1) Enzyme-Linked Immunosorbent Assay (ELISA)

Endpoint antibodies titers against full-length PfCSP (PfCSP-FL) were assessed by ELISA for serum samples collected on Day 21 (pre-boost) and Day 35 (post-boost). Briefly, the RNA constructs were assessed for their ability to induce production of antibodies that may bind to Plasmodium falciparum (Pf) CSP full-length protein (“PfCSP-FL”), using an ELISA assay (see Table 7).

TABLE 7
List of PfCSP recombinant proteins and peptides
assessed by ELISA analyses in the present Example

| | | | | | | SEQ |
|---|---|---|---|---|---|---|
| Protein | Target | AS | | | | ID |
| target | region | sequence | Host | Tag | Sequence | NO.: |
| PfCSP | Full- | 21-376 | Expi293 | His- | MGGAAARLGAVILFVVIVGLHGVRGQEYQCYGS | 77 |
| | length | | cells | tag | SSNTRVLNELNYDNAGTNLYNELEMNYYGKQEN | |
| | | | | (C- | WYSLKKNSRSLGENDDGNNEDNEKLRKPKHKKL | |
| | | | | ter- | KQPADGNPDPNANPNVDPNANPNVDPNANPNV | |
| | | | | minal) | DPNANPNANPNANPNANPNANPNANPNANPNA | |
| | | | | | NPNANPNANPNANPNANPNANPNANPNANPNA | |
| | | | | | INPNANPNVDPNANPNANPNANPNANPNANPNA | |
| | | | | | NPNANPNANPNANPNANPNANPNANPNANPNA | |
| | | | | | NPNANPNANPNANPNANPNKNNQGNGQGHNMP | |
| | | | | | NDPNRNVDENANANSAVKNNNNEEPSDKHIKEY | |
| | | | | | LNKIQNSLSTEWSPCSVTCGNGIQVRIKPGSAN | |
| | | | | | KPKDELDYANDIEKKICKMEKCSS | |

MaxiSorp 96-well plates were coated with 100 ng/well of PfCSP-FL in coating buffer (50 mM sodium carbonate, pH 9.6) and incubated overnight at 4° C. Plates were then blocked with 1% BSA in PBS for 1 h at 37° C. Bound IgG was detected using horseradish peroxidase (HRP)-conjugated goat anti-mouse IgG. Signal was detected after adding the substrate 3,3′,5,5′-Tetramethylbenzidine (TMB) and 25% sulfuric acid to stop the reaction. Optical densities (OD) were read at 450 nm. Reciprocal end titers at day 35, after 2 immunizations, were used as a representative as they showed the highest antibody response.

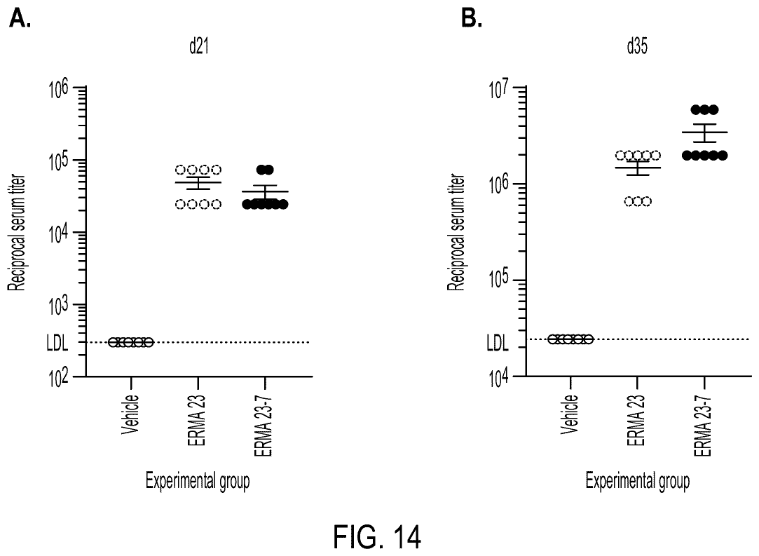

As shown in FIG. 14A and FIG. 14B, immunization with both RNA Construct 23 and variant 7 elicited similar antibody responses against PfCSP on both days analyzed. The second immunization increased the antibody titers against PfCSP by more than 10-fold for both variants, when compared to the titer before the boost (at Day 21). No titers have been detected for control mice (injected with the vehicle).

### (2) Multiplex Assay

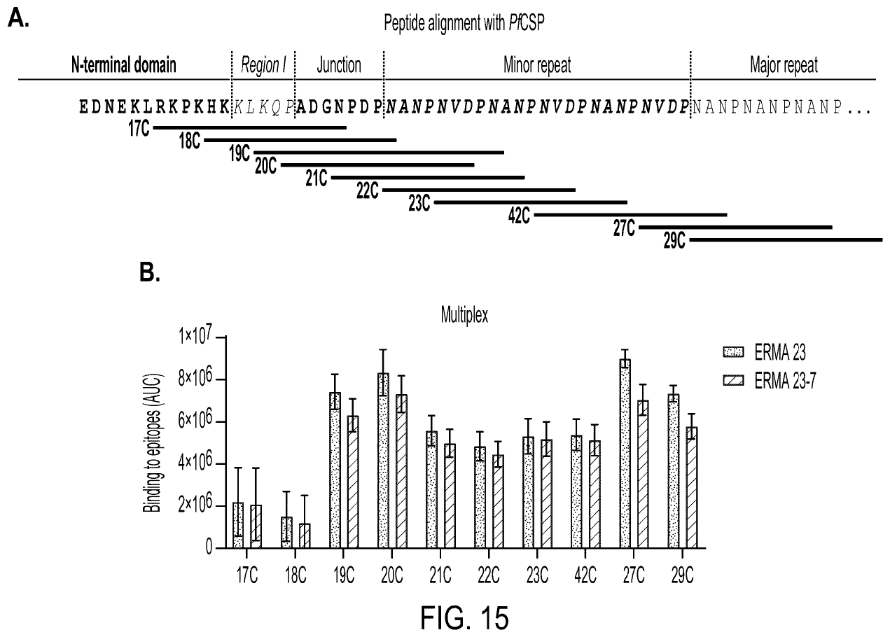

A customized multiplex assay (Meso Scale Discovery, MSD) was employed to quantify the binding of the antibodies present in the serum samples from Day 35 to 10 different PfCSP peptides from the central region of the protein (that spans from the end of the N-terminal domain until the major repeats) (FIG. 15A; Table 8).

Briefly, ten peptides from the central region of PfCSP (PfCSP peptide 17C, 18C, 19C, 20C, 21C, 22C, 23C, 27C, 29C, or 42C) were conjugated with bovine serum albumin (BSA) and then bound to the wells of a 96-well plate, in a specific spot on the well. After incubation with serum from immunized mice, antibodies bound to each specific peptide were detected with a “Sulfo-Tag” conjugated secondary antibody. A multiplex reader instrument (MESO QuickPlex SQ 120) was used to quantify the light emitted from the Sulfo-Tag.

TABLE 8
PfCSP peptides used in the multiplex analysis

| Desig- | | | SEQ ID |
|---|---|---|---|
| nation | AS sequence | Sequence | NO.: |
| 17C | 87-101 | CRKPKHKKLKQPADGN | 78 |
| 18C | 91-105 | CHKKLKQPADGNPDPN | 79 |
| 19C | 95-113 | CKQPADGNPDPNANPN | 80 |
| | | VDPN | |
| 20C | 97-111 | CPADGNPDPNANPNVD | 81 |
| 21C | 101-115 | CNPDPNANPNVDPNAN | 82 |
| 22C | 105-123 | CNANPNVDPNANPNVD | 83 |
| 23C | 109-123 | CNVDPNANPNVDPNAN | 84 |
| 27C | 125-139 | CNVDPNANPNANPNAN | 85 |
| 29C | 129-143; 145- | CNANPNANPNANPNAN | 86 |
| | 159; 161-175; | | |
| | 177-191; 201- | | |
| | 215; 217-231; | | |
| | 233-247; | | |
| | 249-263 | | |
| 42C | 185-199 | CNVDPNANPNVDPNAN | 87 |

This assay confirmed that the antibodies generated upon immunization with RNA Construct 23 or with variant 7 were able to bind the central region of the PfCSP protein, including three well-described neutralizing epitopes: 21C that corresponds to the CIS43-binding epitope, 22C that is the binding sequence of L9, and 29C, which is the main epitope for Ab317 (Jelínková et al. 2022; Langowski et al. 2022; Francica et al. 2021). Both RNA Construct 23 and variant 7 showed a similar pattern of binding to the different epitopes: the highest binding was observed for peptides 19C and 20C, that include the junctional region, and to 27C and 29C, that include the terminal region of minor repeats, and the major repeats region (FIG. 15B).

### (3) Sporozoite ELISA

RNA constructs were assessed for their ability to induce production of antibodies that bind to native CSP antigen from Plasmodium falciparum (Pf) sporozoite lysates, using a sporozoite ELISA assay. Specifically, 384-well plates were coated with non-denatured total protein lysate from P. falciparum NF54 salivary gland sporozoites, equivalent to 250 sporozoites per well. Following blocking, serially diluted serum samples (6 dilutions per sample) were added to the corresponding wells. The binding of the antibodies present in the serum samples to the native PfCSP protein in the wells was detected using an AP-conjugated secondary antibody followed by luminescent quantification.

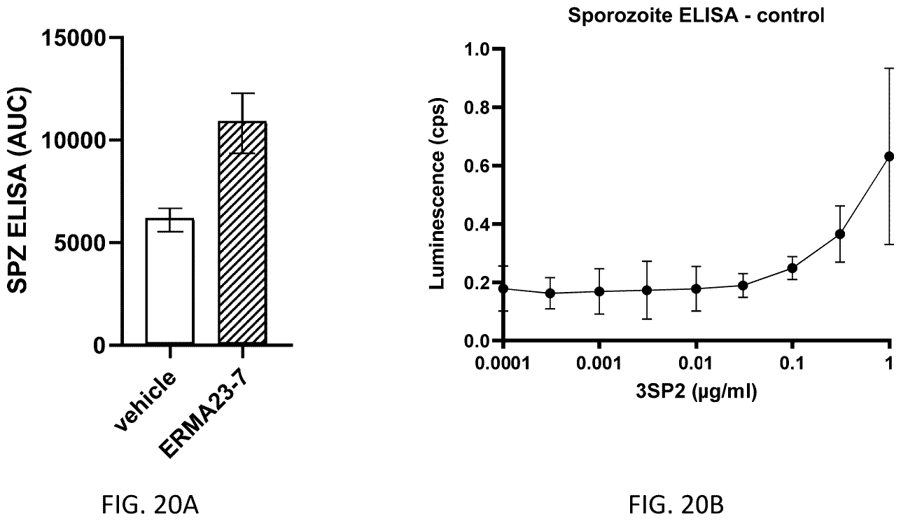

As shown in FIG. 20A, RNA construct 23-7 effectively induced the production of antibodies that bind native PfCSP above the background level, obtained with the serum from vehicle-injected mice. FIG. 20B shows the binding of murine anti-CSP mAb3SP2, used as positive control in the assay.

### (4) Traversal Assay

RNA constructs are assessed for their ability to induce production of antibodies that have an inhibitory effect on Plasmodium falciparum (Pf) sporozoites traversal. Briefly, HC-04 cells, a human hepatocyte cell line, are seeded into the plates and incubated for 24 h at 5% CO2 and 37° C. Freshly isolated P. falciparum salivary gland sporozoites are pre-incubated with serially diluted (1:20, 1:80 and 1:320) serum samples from mice immunized with the formulated RNAs encoding different CSP-based constructs. Then, sporozoites are added to the HC-04 cells in a multiplicity of infection (MOI) of 1:1, in the presence of the impermeable dye dextran-rhodamine. As a positive control for inhibition, sporozoites are pre-treated with mAb317, an antibody that targets the NANP repeats and has a well-established impact on sporozoite traversal and infectivity. Non-treated sporozoites are used as a negative control for inhibition. The ability of P. falciparum sporozoites to traverse cells is quantified by determining the % of cells that incorporate dextran-rhodamine, by fluorescence microscopy. Sporozoite traversal of sporozoites pre-incubated with serum samples from mice injected with the vehicle was set as 0% traversal inhibition.

### (5) Inhibition of Liver Stage Development Assay (ILSDA)

Freshly isolated salivary gland sporozoites are pre-incubated with serially diluted serum samples from mice immunized with the formulated RNAs encoding different CSP-based constructs. Then, these sporozoites are added to primary human hepatocytes previously seeded on a glass-bottom black 96-well plate. After centrifugation to facilitate infection, cells are incubated for 4 days at 5% CO2 and 37° C. After fixation, parasite cytoplasm is stained with anti-PfHsp70 and DNA (from hepatocytes and from parasites) is stained with DAPI. Sporozoites incubated with serum from mice injected with vehicle are used as a negative control for inhibition. The ability of P. falciparum sporozoites to infect hepatocytes is quantified by determining the % of cells with parasites inside, by fluorescence microscopy. Parasite development is also assessed by measuring the area of the parasites (stained with anti-PfHsp70).

(6) Total Binding and Dissociation from PfCSP Full Length Protein or Specific Peptides by SPR

Binding and dissociation of the antibodies present in serum from mice immunized with RNA constructs to biotinylated full length PfCSP, junction+minor repeats peptide, or major repeats peptide (see Table 9 below) will be determined by Surface Plasmon Resonance (SPR). Polypeptides used in the SPR measurements are provided in the table below. Briefly, biotinylated full length PfCSP, junction+minor repeats peptide and major repeats peptide are each immobilized to a different flow cell of a Biacore T200 instrument, while the fourth flow cell is left empty as reference. Serum samples are diluted in analysis buffer (10 mM HEPES (pH 7.4), 150 mM NaCl, 3 mM EDTA, 0.05% Tween 20, 0.22 μm filtered) and run using a flow rate of 10 μL/min for interaction analysis of the analyte (association and dissociation). Association of the antibodies in the serum is measured for 5 min, while dissociation is measured for 15 min. Analyzes are performed in triplicates with individually prepared dilutions. Buffer blanks are implemented regularly and used for referencing. Evaluation of serum sample binding data is performed with regard to two parameters: a) height of binding signals, as relative comparison of the titer, and b) assessment of antibody:antigen complex stability based on dissociation, by calculating the binding signal after 5 min and 15 min, which is determined by calculating the dissociation relative to the maximal signal (% residual response). The higher the % residual response value the higher the complex stability.

TABLE 9
Polypeptides SPR measurements

| | As | | | SEQ |
|---|---|---|---|---|
| Designa- | se- | | | ID |
| tion | quence | Tag | Sequence | NO.: |
| PfCSP- | 1-397 | His- | MMRKLAILSVSSFLFVEALFQEYQCYGSSSNTRVLNELNYDNA | 88 |
| FL | | tag | GTNLYNELEMNYYGKQENWYSLKKNSRSLGENDDGNNEDNEKL | |
| | | (C- | RKPKHKKLKQPADGNPDPNANPNVDPNANPNVDPNANPNVDPN | |
| | | terminal) | ANPNANPNANPNANPNANPNANPNANPNANPNANPNANPNANP | |
| | | | NANPNANPNANPNANPNANPNANPNVDPNANPNANPNANPNAN | |
| | | | PNANPNANPNANPNANPNANPNANPNANPNANPNANPNANPNA | |
| | | | NPNANPNANPNANPNKNNQGNGQGHNMPNDPNRNVDENANANS | |
| | | | AVKNNNNEEPSDKHIKEYLNKIQNSLSTEWSPCSVTCGNGIQV | |
| | | | RIKPGSANKPKDELDYANDIEKKICKMEKCSSVFNVVNSSIGL | |
| | | | IMVLSFLFLN | |
| Junction | 81-128 | biotin | EDNEKLRKPKHKKLKQPADGNPDPNANPNVDPNANPNVDPNAN | 89 |
| repeats | | | PNVDP | |
| + minor | | | | |
| Major | multiple | biotin | NANPNANPNANPNANPNANPNANP | 90 |
| repeats | | | | |

### (7) FluoroSpot Assay

Provided polyribonucleotides can be assessed for their ability to induce T-cell responses upon stimulation with peptides from Pf antigens. In some embodiments, a polyribonucleotide is determined to induce a useful immune response if splenocytes from a subject (e.g., a mouse) immunized with such construct, following incubation with peptide(s) as described herein, exhibit T-cell secretion of one or more pro-inflammatory cytokines (e.g., IFN-γ, TNF-α, or IL-2) in a FluoroSpot Assay, using the IFN-γ/IL-2/TNF-α FluoroSpotPLUS kit according to the manufacturer's instructions (Mabtech).

Splenocytes were freshly isolated from the spleens of mice immunized with RNA constructs and resuspended in culture medium (RPMI1640+10% heat-inactivated fetal calf serum (FCS)+1% non-essential amino acids (NEAA)+1% Sodium Pyruvate+1% HEPES+0.5% Penicillin/Streptomycin, +0.1% β-Mercaptoethanol). After determining cell concentration for each sample using a cell counter, a total of 5×10⁵ splenocytes were added to each well and restimulated ex vivo overnight at 37° C. with the appropriate target peptide pool, e.g., as indicated in Table 10 below or with controls (negative control: Trp1, 2 μg/mL; positive control: concanavalin A, 2 μg/mL). On the following day, anti-IFN-γ, anti-IL-2 and anti-TNF-α antibodies were added to the wells to detect production of these cytokines and then secondary antibodies conjugated with different fluorophores were added. Fluorescent spots were counted using a Mabtech IRIS FluoroSpot plate reader.

For assessing specific CD4 and CD8 T cell responses, CD4 and CD8 isolation kits (Mylteni) were employed to specifically isolate these cells from all the cells isolated from the spleens, according to the manufacturer's instructions. Shortly, splenocytes were resuspended in MACS buffer, the biotin-labelled antibody cocktail was added to the cells, mixed and incubated for 5 minutes at 2-8° C. Then, MACS buffer was added again, followed by anti-biotin microbeads and an incubation for 10 minutes at 2-8° C. Columns were conditioned by adding MACS buffer to them and allowing the MACS buffer to go through the columns completely. Cell suspensions were then added to the columns, followed by a wash with MACS buffer, and the flow-through containing the population of interest (because a negative selection method is used) was collected in a tube. MACS-isolated CD4 and CD8 T cells were counted, and 1×105 cells were added per well of a fluorospot plate. Cells were then restimulated ex vivo overnight at 37° C. with appropriate target peptide pools, e.g., as indicated in Table 10 below or with controls (negative control: Trp1, 2 μg/mL; positive control: concanavalin A, 2 μg/mL), in the presence of antigen-presenting cells, which are added separately. On the following day, anti-IFN-γ, anti-IL-2 and anti-TNF-α antibodies were added to the wells to detect production of these cytokines and then secondary antibodies conjugated with different fluorophores were added. Fluorescent spots were counted using a Mabtech IRIS FluoroSpot plate reader.

TABLE 10
Peptides used for splenocyte stimulation in the FluoroSpot Assay

| | As | | | | SEQ ID |
|---|---|---|---|---|---|
| Designation | sequence | Sequence | Presentation | Comments | NO.: |
| PfCSP | 1-397 | MMRKLAILSVSSFLFVEALFQEYQCYG | MHC-I | Pool of 65 | 91 |
| FL_pep | | SSSNTRVLNELNYDNAGTNLYNELEM | and MHC-II | peptides | |
| (overlapping | | NYYGKQENWYSLKKNSRSLGENDDG | | (15 mer, | |
| peptides) | | NNEDNEKLRKPKHKKLKQPADGNPDP | | overlapping | |
| | | NANPNVDPNANPNVDPNANPNVDPN | | by 11) | |
| | | ANPNANPNANPNANPNANPNANPNA | | | |
| | | NPNANPNANPNANPNANPNANPNAN | | | |
| | | PNANPNANPNANPNANPNVDPNANP | | | |
| | | NANPNANPNANPNANPNANPNANPN | | | |
| | | ANPNANPNANPNANPNANPNANPNA | | | |
| | | NPNANPNANPNANPNANPNKNNQGN | | | |
| | | GQGHNMPNDPNRNVDENANANSAVK | | | |
| | | NNNNEEPSDKHIKEYLNKIQNSLSTE | | | |
| | | WSPCSVTCGNGIQVRIKPGSANKPKD | | | |
| | | ELDYANDIEKKICKMEKCSSVFNVVN | | | |
| | | SSIGLIMVLSFLFLN | | | |

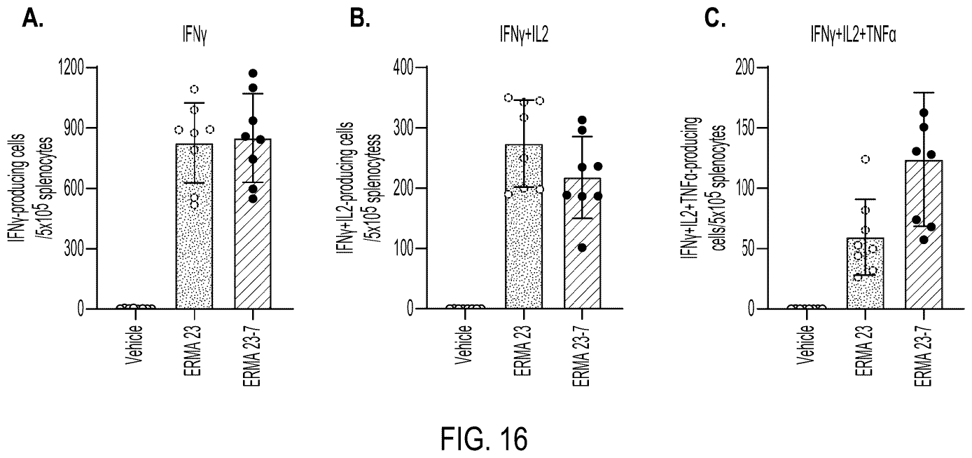

Upon incubation with overlapping peptides spanning the full-length PfCSP protein, splenocytes from mice immunized with RNA Construct 23 or variant 7 produced similar levels of pro-inflammatory cytokines, such as IFNγ, IL-2 and TNFα, alone or in different combinations (FIG. 16). Such a response was not observed in splenocytes from mice injected with vehicle alone.

### Example 6: In Vivo Immunogenicity of an Exemplary Polyribonucleotide Encoding Full-Length Plasmodium CSP Polypeptide Construct Variant 7

The immunogenicity of variant 7 was further evaluated in HLA-A02 mice. Briefly, HLA-A02 mice were injected IM on Days 0 and 21 with 1 μg of variant 7. The vehicle control group (n=5) was injected with saline on the same days. Blood samples were collected on Days 21 and 35 for analysis of antibody titers in the serum. At the end of the experiment (Day 35), mice were sacrificed and splenocytes were isolated and used for T-cell activation analyses.

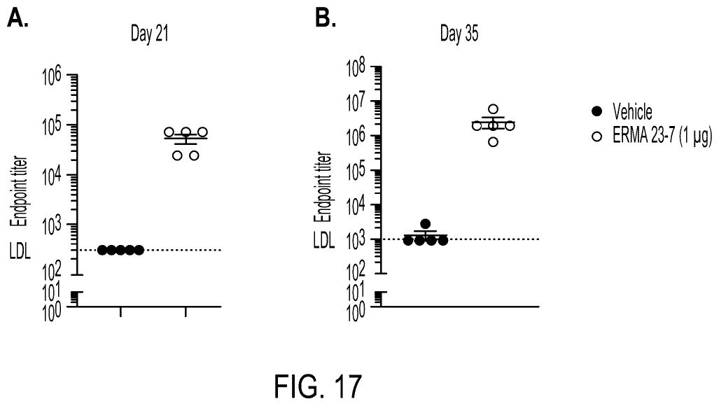

Endpoint antibodies titers against PfCSP-FL were assessed by ELISA as described above for serum samples collected on Day 21 (pre-boost) and Day 35 (post-boost). As shown in FIG. 17A and FIG. 17B, immunization with variant 7 elicited an antibody response against PfCSP that was detectable on both days analyzed, with the titers at Day 35 being >10× higher than those at Day 21.

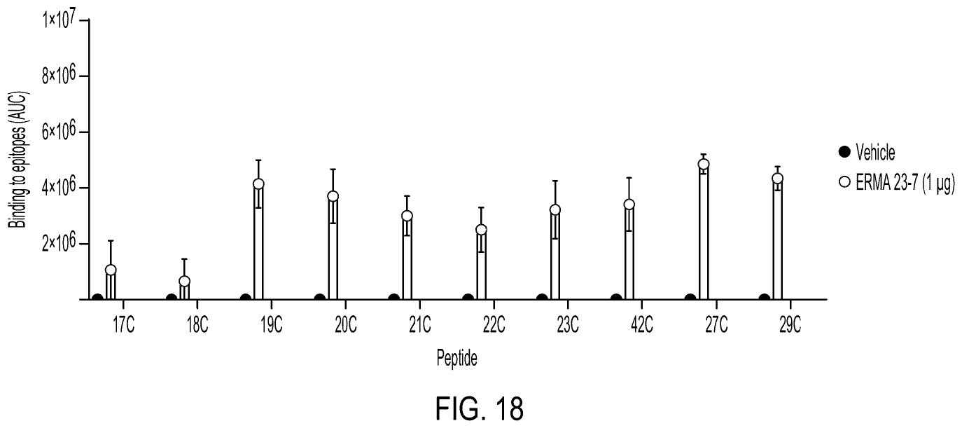

The multiplex assay as described above was employed to quantify the binding of the antibodies present in the serum samples of the immunized HLA-A02 mice from Day 35. The results are shown in FIG. 18. The antibodies generated upon immunization with variant 7 showed a pattern of binding to the central region of PfCSP (FIG. 18). The highest binding was observed for peptides 19C and 20C, which include the junction, and to 27C and 29C, which include the terminal region of minor repeats, and the major repeats region (FIG. 18).

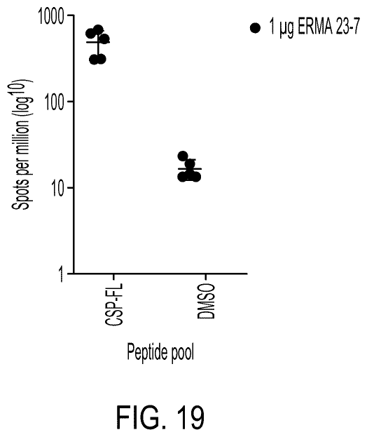

Finally, T-cell responses were assessed as described above. An increase in the level of antigen-specific IFNγ-T-cell responses was observed upon administration of variant 7 (FIG. 19). These results demonstrate that variant 7 was able to elicit an immunogenic response in vivo in mice.

### Example 7: In Vivo Immunogenicity in Humans Following Administration of an Exemplary Polyribonucleotides Encoding Plasmodium CSP Polypeptide Construct

The present Example demonstrates that exemplary polyribonucleotides encoding different malarial polypeptide, as described herein, can be immunogenic in vivo in humans. Specifically, the present Example provides a randomized, dose-escalation trial for the evaluation of safety, tolerability, and immunogenicity of a formulated RNA construct as provided herein. The exemplary formulated RNA constructs to be assessed in this Example are RNA Construct 23 variant 7 (referred to as “variant 7”).

### Trial Inclusion Criteria

Subjects to be included in a trial as described in this Example:
i. Are aged 18 to 55 years.
ii. Have a body mass index over 18.5 kg/m² and under 35 kg/m² and weigh at least 45 kg at an initial visit (“Visit 0”).
iii. Are healthy, in the clinical judgment of a health practitioner (e.g., a trial investigator) based on reported medical history data, physical examination, 12-lead electrocardiogram (ECG), vital signs, and clinical laboratory test outcomes. Healthy subjects with pre-existing stable disease (e.g., obesity, hypertension, etc.), defined as disease but not requiring significant change in therapy or hospitalization for worsening disease during the three months (e.g., 90 days) before Visit 0, can be included.
iv. Agree not to enroll in another trial with an investigational medicinal product (“IMP”) starting from Visit 0 and until 12 weeks after receiving a third dose of a formulated RNA construct or a combination of formulated RNA constructs.
v. Have not traveled and agree not to travel to a malaria-endemic region, as defined per Centers for Disease Control and Prevention (CDC) starting 6 months before Visit 0 and continuously until 28 days after receiving a third dose of a formulated RNA construct or a combination of formulated RNA constructs.
vi. Have a negative blood test result for human immunodeficiency virus (HIV)-1 and -2 at Visit 0.
vii. Have a negative blood test result for SARS-CoV-2 at Visit 0.
viii. Have a negative blood test result for Hepatitis B surface antigen (HBsAg) at Visit 0 and negative anti Hepatitis C virus (anti-HCV) antibodies, or negative HCV PCR test result if the anti-HCV is positive at Visit 0.
ix. If a subject has childbearing potential, the subject has a negative serum beta-human chorionic gonadotropin (P-HCG) pregnancy test result at Visit 0 and negative urine pregnancy test results before each administration of a formulated RNA construct or a combination of formulated RNA constructs. Subjects born female who are postmenopausal or permanently sterilized are not be considered to have childbearing potential.
x. If a subject has childbearing potential, the subject agree to practice a highly effective form of contraception starting at Visit 0 and continuously until 90 days after receiving a third dose of a formulated RNA construct or a combination of formulated RNA constructs.
xi. If a subject has childbearing potential, the subject agree not to donate or cryopreserve eggs (ova, oocytes) for the purposes of assisted reproduction during trial, starting at Visit 0 and continuously until 90 days after receiving a third dose of a formulated RNA construct or a combination of formulated RNA constructs.
xii. If a subject is male, does not have had a vasectomy and are sexually active with partners of childbearing potential agree to use condoms and to practice a highly effective form of contraception with their sexual partners of childbearing potential during the trial, starting at Visit 0 and continuously until 90 days after receiving a third dose of a formulated RNA construct or a combination of formulated RNA constructs.
xiii. If a subject is male, will refrain from sperm donation, starting at Visit 0 and continuously until 90 days after receiving a third dose of a formulated RNA construct or a combination of formulated RNA constructs.

### Trial Exclusion Criteria

Subjects with the following are excluded from a trial described in the present Example:
i. A history of Plasmodium parasitemia (any species) based on subject-reported medical history.
ii. A prior residence for greater than 6 months continuously in a malaria-endemic region as defined per CDC at any point during their lifetime.
iii. Participation in breastfeeding or an intention to become pregnant or to father children starting with Visit 0 and continuously until 90 after receiving a third dose of a formulated RNA construct or a combination of formulated RNA constructs.
iv. A history of any serious adverse reactions to vaccines or to vaccine components such as lipids, and including history of anaphylaxis and related symptoms such as hives, respiratory difficulty, angioedema, and/or abdominal pain (not excluded from participation: a subject who had an anaphylactic adverse reaction to pertussis vaccine as a child).
v. Existence or history of the following medical conditions:
   a. Uncontrolled or moderate or severe respiratory diseases (e.g., asthma, chronic obstructive pulmonary disease); symptoms of asthma severity as defined in the US National Asthma Education and Prevention Program Expert Panel report, 2020—e.g., exclude a volunteer who:
      i. Uses a short-acting rescue inhaler (typically a beta 2 agonist) daily, or
      ii. Uses high dose inhaled corticosteroids (per American Academy of Allergy Asthma & Immunology), or
      iii. In the past year has either of the following:
         1. Greater than one exacerbation of symptoms treated with oral/parenteral corticosteroids; or
         2. Needed hospitalization, or intubation for asthma;
   b. Diabetes mellitus type 1 or type 2, including cases controlled with diet alone or elevated hemoglobin A1C (HbA1c) ≥6.5% at screening (not excluded: history of isolated gestational diabetes);
   c. Hypertension:
      i. If a person has a history of hypertension, or elevated blood pressure detected during screening, exclude for blood pressure that is not well controlled. Well controlled blood pressure is defined as consistently 140 mm Hg systolic and 590 mm Hg diastolic, with or without medication, with only isolated, brief instances of higher readings, which must be 5150 mm Hg systolic and 5100 mm Hg diastolic at screening and enrollment;
   d. Malignancy within 5 years of screening, excluding localized basal or squamous cell skin cancer;
   e. Any current or history of cardiovascular diseases, (e.g., myocarditis, pericarditis, myocardial infarction, congestive heart failure, cardiomyopathy or clinically significant arrhythmias), unless such disease is not considered relevant for participation in this trial in a health practitioner's (e.g., investigator's) judgment;
   f. An abnormal screening ECG (i.e., showing the corrected QT interval by Fridericia (QTcF) >150 ms; significant ST-T wave changes suggestive of myocardial ischemia or of an acute or indeterminate-age myocardial infarction; left ventricular hypertrophy; any non-sinus rhythm including isolated premature ventricular contractions; complete right or left bundle branch block [QRS >120 ms]; second- or third-degree atrioventricular [AV] block); or other clinically significant abnormalities on the ECG at the investigator's discretion;
   g. Bleeding disorder diagnosed by a doctor (e.g., factor deficiency, coagulopathy, or platelet disorder requiring special precautions); or
   h. Seizure disorder: History of seizure(s) within past 3 years. Also exclude if volunteer has used medications in order to prevent or treat seizure(s) at any time within the past 3 years.
vi. Documented major psychiatric illness, including bipolar disorder, major depressive disorder, schizophrenia, autism, and attention deficit-hyperactivity disorder that at the discretion of the investigator could interfere with participation and follow-up as outlined by the trial.
vii. The following diseases associated with immune dysregulation:
   a. Primary immunodeficiencies;
   b. History of solid organ or bone marrow transplantation;
   c. Asplenia: any condition resulting in the absence of a functional spleen; or
   d. Existence or history of autoimmune disease including and not limited to thyroid autoimmune disease, multiple sclerosis, psoriasis, etc.
viii. Previous vaccination with an approved or investigational malaria vaccine at any time or having taken part in a human malaria challenge study.
ix. Receipt of any investigational product within 28 days before Visit 0.
x. Any planned non-trial vaccinations starting at Visit 0 and continuously until 28 days after a third dose of a formulated RNA construct or a combination of formulated RNA constructs. Seasonal influenza and COVID-19 vaccines are allowed; however, they should be administered at least 14 days before or 28 days after any IMP administration. Emergency vaccinations, such as tetanus, are allowed to be administered when medically indicated.
xi. Received blood/plasma products, monoclonal antibodies or immunoglobulin within 120 days before Visit 1 or planned administration starting at Visit 0 and continuously until Visit 21 (e.g., 365 days after administration of a third dose).
xii. Received allergy treatment with antigen injections within 28 days before and after each IMP administration.
xiii. Current or planned treatment with immunosuppressive therapy, including systemic corticosteroids (if systemic corticosteroids are administered for 14 days at a dose of 20 mg/d of prednisone or equivalent) starting at Visit 0 and continuously until a third dose of a formulated RNA construct or a combination of formulated RNA constructs. Intraarticular, intrabursal, or topical (skin or eyes) corticosteroids are permitted.
xiv. Have a history of alcohol abuse or drug addiction within 1 year before Visit 0 or have a history (within the past 5 years) of substance abuse, which in the opinion of a health practitioner (e.g., an investigator), could compromise their wellbeing if they participate as a subject in a trial, or that could prevent, limit, or confound the protocol specified assessments.
xv. Any existing condition which may affect vaccine injection and/or assessment of local reactions at the injection site, e.g., tattoos, severe scars, etc.
xvi. Are vulnerable individuals as per International Council for Harmonisation (ICH) E6 definition, i.e., are individuals whose willingness to volunteer in a clinical trial may be unduly influenced by the expectation, whether justified or not, of benefits associated with participation, or of a retaliatory response from senior members of a hierarchy in case of refusal to participate.
xvii. Any screening hematology and/or blood chemistry laboratory value that meets the definition of a Grade >2 abnormality or a Grade 1 abnormality at a health practitioner's (e.g., an investigator's) discretion at Visit 0. Subjects with abnormal but not clinically significant parameters not included in the toxicity guidance may be considered eligible at discretion of a health practitioner (e.g., an investigator).
xviii. Prior residence for greater than 6 months in a malaria endemic region.
xix. Sickle cell disease.

### Trial—Variant 7

RNA Construct 23-7 is evaluated in different dose combinations in a 3 dose schedule to select a safe, tolerable, and immunogenic dose combination, and to assess the impact of third dose on immunogenicity.

To assess safety, tolerability, and immunogenicity of RNA Construct 23-7, subjects are divided into cohorts. Cohorts are randomized 5:1 active: placebo. Evaluation uses a staggered dose-escalation schema with sentinel subjects in all cohorts. Different cohorts receive different doses of RNA Construct 23-7. Table 11 provides an overview of the dose combinations used. A placebo cohort that receives isotonic NaCl solution (0.9%) is also assessed.

TABLE 11
Dose Combinations

| | Dose level (μg) RNA | Sample size |
|---|---|---|
| Cohort | Construct 23-7 | (active:placebo) |
| 1 | 10 | 10:2 |
| 2 | ≤30 | 10:2 |
| 3 | ≤70 | 10:2 |
| 4 | ≤100 | 10:2 |

A first dose of RNA Construct 23-7 is administered to subject on Day 1. Approximately eight weeks later, a second dose of RNA Construct 23-7 is administered to the subject. A third dose of RNA Construct 23-7 is administered approximately 18 weeks after the second dose.

### Post-Trial Assessments

Subjects are assessed for the following primary outcome measures after each dose of a formulated RNA Construct 23-7:
i. Frequency of solicited local reactions at the injection site (e.g., pain, erythema/redness and/or induration/swelling) recorded up to 7 days after each dose;
ii. Frequency of solicited systemic reactions (vomiting, diarrhea, headache, fatigue, muscle/joint pain, and fever) recorded up to 7 days after each dose;
iii. Frequency of subjects with at least one AE occurring until 28 days after each dose; and
iv. Frequency of subjects with at least one medically attended adverse event (MAAE) occurring until 28 days after each dose.

Subjects are assessed to determine a frequency of subjects in each cohort with at least one SAE occurring until 24 weeks after a third dose.

Descriptive statistics on antibody levels (e.g., anti-CSP antibodies) in a subject can be assessed at various time points. For example, levels of antigen-specific serum and/or plasma antibodies can be assessed using ELISA or similar assays. Additionally, the functionality and/or avidity of serum and/or plasma antibodies are evaluated.

Other immune responses of subjects following administration of one or more doses are also assessed. For instance, CD4+ and CD8+ T-cell responses to antigens in a formulated RNA construct can be measured using polychromatic flow cytometry. Vaccine-induced plasmablasts and/or memory B cells can be measured using flow cytometry, and levels of inflammatory markers, chemokines, and/or cytokines can be measured.

RNA expression patterns in subjects following administration of one or more doses can be assessed using, e.g., RNASeq technology.

### EQUIVALENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A polyribonucleotide comprising a coding sequence that encodes a full-length Plasmodium CSP polypeptide,
wherein the full-length Plasmodium CSP polypeptide comprises a Plasmodium CSP N-terminal domain, and
wherein the coding sequence has an adenine content that is between 35% and 42%, and the portion of the coding sequence that encodes the Plasmodium CSP N-terminal domain has an adenine content that is at least 35%.

2. The polyribonucleotide of claim 1, wherein the portion of the coding sequence that encodes the Plasmodium CSP N-terminal domain has an adenine content that is between 35% and 45%.

3. The polyribonucleotide of claim 1 or 2, wherein the portion of the coding sequence that encodes the Plasmodium CSP N-terminal domain has an adenine content that is between 36% and 42%.

4. The polyribonucleotide of any one of claims 1-3, wherein the coding sequence has an adenine content that is between that is between 35% and 36.5%.

5. The polyribonucleotide of any one of claims 1-4, wherein the coding sequence has a uracil content that is between 14% and 20%, and the portion of the coding sequence that encodes the Plasmodium CSP N-terminal domain has a uracil content that is at least 10%.

6. The polyribonucleotide of any one of claims 1-5, wherein the coding sequence has a guanine content that is between 15% and 19.5%, and the portion of the coding sequence encoding a Plasmodium CSP N-terminal domain has a guanine content that is less than 27%.

7. The polyribonucleotide of any one of claims 1-6, wherein the coding sequence has a cytosine content that is between 22% and 31%, and the portion of the coding sequence that encodes the Plasmodium CSP N-terminal domain has a cytosine content that is less than 28%.

8. The polyribonucleotide of any one of claims 1-7, wherein the full-length Plasmodium CSP polypeptide comprises:
(i) a secretory signal,
(ii) the Plasmodium CSP N-terminal domain, wherein the Plasmodium CSP N-terminal domain comprises an N-terminal region, an N-terminal end region, and a junction region,
(iii) a Plasmodium CSP central domain comprising a minor repeat region and a major repeat region, and
(iv) a Plasmodium CSP C-terminal domain comprising a C-terminal region and a transmembrane region.

9. The polyribonucleotide of claim 8, wherein the secretory signal comprises or consists of a Plasmodium secretory signal, preferably a Plasmodium CSP secretory signal.

10. The polyribonucleotide of any one of claims 1-9, wherein the full-length Plasmodium CSP polypeptide comprises an amino acid sequence of SEQ ID NO: 1.

11. The polyribonucleotide of any one of claims 1-10, wherein Plasmodium is Plasmodium falciparum, preferably Plasmodium falciparum isolate 3D7.

12. The polyribonucleotide of any one of claims 8-11, wherein the portion of the coding sequence encoding the Plasmodium CSP N-terminal domain comprises or consists of a sequence according to any one of SEQ ID NOs: 11, 13, 15, and 17.

13. The polyribonucleotide of any one of claims 1-12, wherein the coding sequence comprises or consists of a sequence according to any one of SEQ ID NOs: 50, 52, 54, 56, 58, 60, and 62.

14. The polyribonucleotide of any one of claims 1-13, wherein the coding sequence comprises or consists of a sequence according to SEQ ID NO: 52.

15. The polyribonucleotide of any one of claims 1-13, wherein the polyribonucleotide comprises or consists of a sequence according to SEQ ID NO: 54.

16. An RNA construct comprising in 5′ to 3′ order:
(i) a 5′ UTR that comprises or consists of a modified human alpha-globin 5′-UTR;
(ii) a polyribonucleotide of any one of claims 1-15;
(iii) a 3′ UTR that comprises or consists of a first sequence from the amino terminal enhancer of split (AES) messenger RNA and a second sequence from the mitochondrial encoded 12S ribosomal RNA; and
(iv) a polyA tail sequence.

17. The RNA construct of claim 16, further comprising a 5′ cap.

18. A composition comprising one or more polyribonucleotides of any one of claims 1-15 or one or more RNA constructs of claim 16 or 17.

19. The composition of claim 18, wherein the composition further comprises lipid nanoparticles, polyplexes (PLX), lipidated polyplexes (LPLX), or liposomes,
wherein the one or more polyribonucleotides are fully or partially encapsulated within the lipid nanoparticles, polyplexes (PLX), lipidated polyplexes (LPLX), or liposomes.

20. A pharmaceutical composition comprising the composition of claim 18 or 19 and at least one pharmaceutically acceptable excipient.

21. A method comprising administering one or more doses of the pharmaceutical composition of claim 20 to a subject.

22. The pharmaceutical composition of claim 20 for use in the treatment and/or prevention of a malaria infection comprising administering one or more doses of the pharmaceutical composition to a subject.

23. A combination comprising:
(i) a first pharmaceutical composition comprising a first polyribonucleotide, wherein the first polyribonucleotide is a polyribonucleotide according to any one of claims 1-15; and
(ii) a second pharmaceutical composition comprising a second polyribonucleotide, wherein the second polyribonucleotide encodes a second polypeptide, and the second polypeptide comprises one or more Plasmodium T-cell antigens.

24. A method comprising administering a combination of claim 23 to a subject.


SEQUENCE LISTING (91 sequences)

SEQ ID NO: 1 - protein, 397 aa, unidentified
  source 1..397: note = Plasmodium falciparum
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 2 - DNA, 1194 bp, unidentified
  source 1..1194: mol_type = other DNA; note = Plasmodium falciparum
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaaccccaatgcaaatcctaatgcaaacccaaatgcaaacccaaacgtagatcctaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacagtgctgtaaaaaataataataacgaagaaccaagtgataagcacataaaagaatatttaaacaaaatacaaaattctctttcaactgaatggtccccatgtagtgtaacttgtggaaatggtattcaagttagaataaagcctggctctgctaataaacctaaagacgaattagattatgcaaatgatattgaaaaaaaaatttgtaaaatggaaaaatgttccagtgtgtttaatgtcgtaaatagttcaataggattaataatggtattatccttcttgttccttaattag

SEQ ID NO: 3 - RNA, 1194 nt, unidentified
  source 1..1194: mol_type = other RNA; note = Plasmodium falciparum
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaaccccaatgcaaatcctaatgcaaacccaaatgcaaacccaaacgtagatcctaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacagtgctgtaaaaaataataataacgaagaaccaagtgataagcacataaaagaatatttaaacaaaatacaaaattctctttcaactgaatggtccccatgtagtgtaacttgtggaaatggtattcaagttagaataaagcctggctctgctaataaacctaaagacgaattagattatgcaaatgatattgaaaaaaaaatttgtaaaatggaaaaatgttccagtgtgtttaatgtcgtaaatagttcaataggattaataatggtattatccttcttgttccttaattag

SEQ ID NO: 4 - protein, 18 aa, unidentified
  source 1..18: note = Plasmodium falciparum
MMRKLAILSVSSFLFVEA

SEQ ID NO: 5 - DNA, 54 bp, synthetic construct
  source 1..54: mol_type = other DNA
atgatgcggaagctggccatcctgagcgtgtccagcttcctgtttgtggaagcc

SEQ ID NO: 6 - RNA, 54 nt, synthetic construct
  source 1..54: mol_type = other RNA
atgatgcggaagctggccatcctgagcgtgtccagcttcctgtttgtggaagcc

SEQ ID NO: 7 - protein, 86 aa, unidentified
  source 1..86: note = Plasmodium falciparum
LFQEYQCYGSSSNTRVLNELNYDNAGTNLYNELEMNYYGKQENWYSLKKNSRSLGENDDGNNEDNEKLRKPKHKKLKQPADGNPDP

SEQ ID NO: 8 - DNA, 258 bp, synthetic construct
  source 1..258: mol_type = other DNA
ctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcct

SEQ ID NO: 9 - RNA, 258 nt, synthetic construct
  source 1..258: mol_type = other RNA
ctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcct

SEQ ID NO: 10 - DNA, 258 bp, synthetic construct
  source 1..258: mol_type = other DNA
ttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatcca

SEQ ID NO: 11 - RNA, 258 nt, synthetic construct
  source 1..258: mol_type = other RNA
ttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatcca

SEQ ID NO: 12 - DNA, 258 bp, synthetic construct
  source 1..258: mol_type = other DNA
ctgtttcaggaataccagtgctacggatcttcttctaatacaagagtgctgaatgaactgaattacgataatgctggaacaaatctctacaatgaactggaaatgaattactacggaaaacaggaaaattggtactctctgaagaaaaattctagatctctgggagaaaatgatgatggaaataatgaagataatgaaaaactgagaaaaccaaaacacaagaaactgaagcagccagctgatggaaatccagatcca

SEQ ID NO: 13 - RNA, 258 nt, synthetic construct
  source 1..258: mol_type = other RNA
ctgtttcaggaataccagtgctacggatcttcttctaatacaagagtgctgaatgaactgaattacgataatgctggaacaaatctctacaatgaactggaaatgaattactacggaaaacaggaaaattggtactctctgaagaaaaattctagatctctgggagaaaatgatgatggaaataatgaagataatgaaaaactgagaaaaccaaaacacaagaaactgaagcagccagctgatggaaatccagatcca

SEQ ID NO: 14 - DNA, 258 bp, synthetic construct
  source 1..258: mol_type = other DNA
ctgttccaggaataccagtgctatggctcaagcagcaataccagagtgctgaatgagctgaactacgacaatgccggaacaaacctgtacaacgaactcgagatgaactactatgggaaacaggagaactggtattccctgaagaagaattctcggagtttgggagaaaacgacgatggcaacaatgaggacaatgagaagcttaggaaaccgaaacacaagaagctcaaacaaccagctgatggtaatcctgatccc

SEQ ID NO: 15 - RNA, 258 nt, synthetic construct
  source 1..258: mol_type = other RNA
ctgttccaggaataccagtgctatggctcaagcagcaataccagagtgctgaatgagctgaactacgacaatgccggaacaaacctgtacaacgaactcgagatgaactactatgggaaacaggagaactggtattccctgaagaagaattctcggagtttgggagaaaacgacgatggcaacaatgaggacaatgagaagcttaggaaaccgaaacacaagaagctcaaacaaccagctgatggtaatcctgatccc

SEQ ID NO: 16 - DNA, 258 bp, synthetic construct
  source 1..258: mol_type = other DNA
ctgttccaggaataccagtgctatgggtcatcttccaataccagagtgctgaatgagctgaattacgacaatgccggaacaaacctgtacaacgaactggagatgaactactatggcaagcaagagaactggtatagcctcaagaagaatagcaggagtttgggtgaaaacgatgatggcaacaatgaggacaacgagaaacttcggaaacccaaacacaagaagctgaagcagcccgccgacggcaatcccgatcct

SEQ ID NO: 17 - RNA, 258 nt, synthetic construct
  source 1..258: mol_type = other RNA
ctgttccaggaataccagtgctatgggtcatcttccaataccagagtgctgaatgagctgaattacgacaatgccggaacaaacctgtacaacgaactggagatgaactactatggcaagcaagagaactggtatagcctcaagaagaatagcaggagtttgggtgaaaacgatgatggcaacaatgaggacaacgagaaacttcggaaacccaaacacaagaagctgaagcagcccgccgacggcaatcccgatcct

SEQ ID NO: 18 - protein, 62 aa, unidentified
  source 1..62: note = Plasmodium falciparum
LFQEYQCYGSSSNTRVLNELNYDNAGTNLYNELEMNYYGKQENWYSLKKNSRSLGENDDGNN

SEQ ID NO: 19 - protein, 12 aa, unidentified
  source 1..12: note = Plasmodium falciparum
EDNEKLRKPKHK

SEQ ID NO: 20 - protein, 12 aa, unidentified
  source 1..12: note = Plasmodium falciparum
KLKQPADGNPDP

SEQ ID NO: 21 - protein, 5 aa, synthetic construct
KLKQP

SEQ ID NO: 22 - protein, 7 aa, synthetic construct
ADGNPDP

SEQ ID NO: 23 - protein, 168 aa, unidentified
  source 1..168: note = Plasmodium falciparum
NANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANP

SEQ ID NO: 24 - DNA, 504 bp, synthetic construct
  source 1..504: mol_type = other DNA
aacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcct

SEQ ID NO: 25 - RNA, 504 nt, synthetic construct
  source 1..504: mol_type = other RNA
aacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcct

SEQ ID NO: 26 - protein, 24 aa, unidentified
  source 1..24: note = Plasmodium falciparum
NANPNVDPNANPNVDPNANPNVDP

SEQ ID NO: 27 - DNA, 72 bp, synthetic construct
  source 1..72: mol_type = other DNA
aacgccaatcctaacgtggaccccaacgctaaccccaatgtggacccaaatgccaatccaaatgtcgatccc

SEQ ID NO: 28 - RNA, 72 nt, synthetic construct
  source 1..72: mol_type = other RNA
aacgccaatcctaacgtggaccccaacgctaaccccaatgtggacccaaatgccaatccaaatgtcgatccc

SEQ ID NO: 29 - protein, 8 aa, synthetic construct
NANPNVDP

SEQ ID NO: 30 - protein, 144 aa, unidentified
  source 1..144: note = Plasmodium falciparum
NANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANP

SEQ ID NO: 31 - DNA, 432 bp, synthetic construct
  source 1..432: mol_type = other DNA
aatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcct

SEQ ID NO: 32 - RNA, 432 nt, synthetic construct
  source 1..432: mol_type = other RNA
aatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcct

SEQ ID NO: 33 - protein, 4 aa, synthetic construct
NANP

SEQ ID NO: 34 - protein, 4 aa, synthetic construct
NVDP

SEQ ID NO: 35 - protein, 6 aa, synthetic construct
NPNANP

SEQ ID NO: 36 - protein, 6 aa, synthetic construct
NANPNA

SEQ ID NO: 37 - protein, 125 aa, unidentified
  source 1..125: note = Plasmodium falciparum
NKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 38 - DNA, 375 bp, synthetic construct
  source 1..375: mol_type = other DNA
aacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaac

SEQ ID NO: 39 - RNA, 375 nt, synthetic construct
  source 1..375: mol_type = other RNA
aacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaac

SEQ ID NO: 40 - protein, 103 aa, unidentified
  source 1..103: note = Plasmodium falciparum
NKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCS

SEQ ID NO: 41 - DNA, 309 bp, synthetic construct
  source 1..309: mol_type = other DNA
aacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagc

SEQ ID NO: 42 - RNA, 309 nt, synthetic construct
  source 1..309: mol_type = other RNA
aacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagc

SEQ ID NO: 43 - protein, 20 aa, unidentified
  source 1..20: note = Plasmodium falciparum
FNVVNSSIGLIMVLSFLFLN

SEQ ID NO: 44 - DNA, 60 bp, synthetic construct
  source 1..60: mol_type = other DNA
ttcaacgtggtcaacagcagcatcggcctgattatggtgctgtccttcctgttcctgaac

SEQ ID NO: 45 - RNA, 60 nt, synthetic construct
  source 1..60: mol_type = other RNA
ttcaacgtggtcaacagcagcatcggcctgattatggtgctgtccttcctgttcctgaac

SEQ ID NO: 46 - protein, 397 aa, synthetic construct; identical to SEQ ID NO: 1

SEQ ID NO: 47 - DNA, 1197 bp, synthetic construct
  source 1..1197: mol_type = other DNA
atgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatga

SEQ ID NO: 48 - RNA, 1197 nt, synthetic construct
  source 1..1197: mol_type = other RNA
atgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatga

SEQ ID NO: 49 - DNA, 1197 bp, synthetic construct
  source 1..1197: mol_type = other DNA
atgatgcggaagctggccatcctgagcgtgtccagcttcctgtttgtggaagccctgttccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatccaaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatga

SEQ ID NO: 50 - RNA, 1197 nt, synthetic construct
  source 1..1197: mol_type = other RNA
atgatgcggaagctggccatcctgagcgtgtccagcttcctgtttgtggaagccctgttccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatccaaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatga

SEQ ID NO: 51 - DNA, 1197 bp, synthetic construct
  source 1..1197: mol_type = other DNA
atgatgcggaagctggccatcctgagcgtgtccagcttcctgtttgtggaagccctgtttcaggaataccagtgctacggatcttcttctaatacaagagtgctgaatgaactgaattacgataatgctggaacaaatctctacaatgaactggaaatgaattactacggaaaacaggaaaattggtactctctgaagaaaaattctagatctctgggagaaaatgatgatggaaataatgaagataatgaaaaactgagaaaaccaaaacacaagaaactgaagcagccagctgatggaaatccagatccaaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatga

SEQ ID NO: 52 - RNA, 1197 nt, synthetic construct
  source 1..1197: mol_type = other RNA
atgatgcggaagctggccatcctgagcgtgtccagcttcctgtttgtggaagccctgtttcaggaataccagtgctacggatcttcttctaatacaagagtgctgaatgaactgaattacgataatgctggaacaaatctctacaatgaactggaaatgaattactacggaaaacaggaaaattggtactctctgaagaaaaattctagatctctgggagaaaatgatgatggaaataatgaagataatgaaaaactgagaaaaccaaaacacaagaaactgaagcagccagctgatggaaatccagatccaaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatga

SEQ ID NO: 53 - DNA, 1197 bp, synthetic construct
  source 1..1197: mol_type = other DNA
atgatgcggaagctggccatcctgagcgtgtccagcttcctgtttgtggaagccctgttccaggaataccagtgctatggctcaagcagcaataccagagtgctgaatgagctgaactacgacaatgccggaacaaacctgtacaacgaactcgagatgaactactatgggaaacaggagaactggtattccctgaagaagaattctcggagtttgggagaaaacgacgatggcaacaatgaggacaatgagaagcttaggaaaccgaaacacaagaagctcaaacaaccagctgatggtaatcctgatcccaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatga

SEQ ID NO: 54 - RNA, 1197 nt, synthetic construct
  source 1..1197: mol_type = other RNA
atgatgcggaagctggccatcctgagcgtgtccagcttcctgtttgtggaagccctgttccaggaataccagtgctatggctcaagcagcaataccagagtgctgaatgagctgaactacgacaatgccggaacaaacctgtacaacgaactcgagatgaactactatgggaaacaggagaactggtattccctgaagaagaattctcggagtttgggagaaaacgacgatggcaacaatgaggacaatgagaagcttaggaaaccgaaacacaagaagctcaaacaaccagctgatggtaatcctgatcccaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatga

SEQ ID NO: 55 - DNA, 1197 bp, synthetic construct
  source 1..1197: mol_type = other DNA
atgatgcggaagctggccatcctgagcgtgtccagcttcctgtttgtggaagccctgttccaggaataccagtgctatgggtcatcttccaataccagagtgctgaatgagctgaattacgacaatgccggaacaaacctgtacaacgaactggagatgaactactatggcaagcaagagaactggtatagcctcaagaagaatagcaggagtttgggtgaaaacgatgatggcaacaatgaggacaacgagaaacttcggaaacccaaacacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatga

SEQ ID NO: 56 - RNA, 1197 nt, synthetic construct
  source 1..1197: mol_type = other RNA
atgatgcggaagctggccatcctgagcgtgtccagcttcctgtttgtggaagccctgttccaggaataccagtgctatgggtcatcttccaataccagagtgctgaatgagctgaattacgacaatgccggaacaaacctgtacaacgaactggagatgaactactatggcaagcaagagaactggtatagcctcaagaagaatagcaggagtttgggtgaaaacgatgatggcaacaatgaggacaacgagaaacttcggaaacccaaacacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatga

SEQ ID NO: 57 - DNA, 1197 bp, synthetic construct
  source 1..1197: mol_type = other DNA
atgatgcggaagctggccatcctgagcgtgtccagcttcctgtttgtggaagccctgtttcaggaataccagtgctacggatcttcttctaatacaagagtgctgaatgaactgaattacgataatgctggaacaaatctctacaatgaactggaaatgaattactacggaaaacaggaaaattggtactctctgaagaaaaattctagatctctgggagaaaatgatgatggaaataatgaagataatgaaaaactgagaaaaccaaaacacaagaaactgaagcagccagctgatggaaatccagatccaaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaataaaaacaatcaaggaaatggacagggacacaatatgccaaatgatccaaatagaaatgtggatgaaaatgcaaatgcaaactctgctgtgaaaaataataataatgaagaaccatctgataagcacatcaaagaatacctgaataaaatccagaattctctgtctacagaatggtctccttgctctgtgacatgcggaaatggaatccaggtgagaatcaaaccaggatctgcaaataaaccaaaagatgaactggattatgcaaatgatattgaaaagaaaatttgcaaaatggaaaaatgcagcagcgtgttcaacgtggtcaacagcagcatcggcctgattatggtgctgtccttcctgttcctgaactgatga

SEQ ID NO: 58 - RNA, 1197 nt, synthetic construct
  source 1..1197: mol_type = other RNA
atgatgcggaagctggccatcctgagcgtgtccagcttcctgtttgtggaagccctgtttcaggaataccagtgctacggatcttcttctaatacaagagtgctgaatgaactgaattacgataatgctggaacaaatctctacaatgaactggaaatgaattactacggaaaacaggaaaattggtactctctgaagaaaaattctagatctctgggagaaaatgatgatggaaataatgaagataatgaaaaactgagaaaaccaaaacacaagaaactgaagcagccagctgatggaaatccagatccaaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaataaaaacaatcaaggaaatggacagggacacaatatgccaaatgatccaaatagaaatgtggatgaaaatgcaaatgcaaactctgctgtgaaaaataataataatgaagaaccatctgataagcacatcaaagaatacctgaataaaatccagaattctctgtctacagaatggtctccttgctctgtgacatgcggaaatggaatccaggtgagaatcaaaccaggatctgcaaataaaccaaaagatgaactggattatgcaaatgatattgaaaagaaaatttgcaaaatggaaaaatgcagcagcgtgttcaacgtggtcaacagcagcatcggcctgattatggtgctgtccttcctgttcctgaactgatga

SEQ ID NO: 59 - DNA, 1197 bp, synthetic construct
  source 1..1197: mol_type = other DNA
atgatgaggaagcttgccatcctgtctgtcagctccttcctctttgtggaggctctgtttcaggaataccagtgctacggatcttcttctaatacaagagtgctgaatgaactgaattacgataatgctggaacaaatctctacaatgaactggaaatgaattactacggaaaacaggaaaattggtactctctgaagaaaaattctagatctctgggagaaaatgatgatggaaataatgaagataatgaaaaactgagaaaaccaaaacacaagaaactgaagcagccagctgatggaaatccagatccaaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatga

SEQ ID NO: 60 - RNA, 1197 nt, synthetic construct
  source 1..1197: mol_type = other RNA
atgatgaggaagcttgccatcctgtctgtcagctccttcctctttgtggaggctctgtttcaggaataccagtgctacggatcttcttctaatacaagagtgctgaatgaactgaattacgataatgctggaacaaatctctacaatgaactggaaatgaattactacggaaaacaggaaaattggtactctctgaagaaaaattctagatctctgggagaaaatgatgatggaaataatgaagataatgaaaaactgagaaaaccaaaacacaagaaactgaagcagccagctgatggaaatccagatccaaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatga

SEQ ID NO: 61 - DNA, 1197 bp, synthetic construct
  source 1..1197: mol_type = other DNA
atgatgagaaaactggcaattctgtctgtgtcttcttttctgtttgtggaagctctgtttcaggaataccagtgctacggatcttcttctaatacaagagtgctgaatgaactgaattacgataatgctggaacaaatctctacaatgaactggaaatgaattactacggaaaacaggaaaattggtactctctgaagaaaaattctagatctctgggagaaaatgatgatggaaataatgaagataatgaaaaactgagaaaaccaaaacacaagaaactgaagcagccagctgatggaaatccagatccaaatgcaaatccaaatgtggacccaaatgcaaatccaaatgtggacccaaatgcaaatccaaatgtggacccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgtggacccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaataaaaacaatcaaggaaatggacagggacacaatatgccaaatgatccaaatagaaatgtggatgaaaatgcaaatgcaaactctgctgtgaaaaataataataatgaagaaccatctgataagcacatcaaagaatacctgaataaaatccagaattctctgtctacagaatggtctccttgctctgtgacatgcggaaatggaatccaggtgagaatcaaaccaggatctgcaaataaaccaaaagatgaactggattatgcaaatgatattgaaaagaaaatttgcaaaatggaaaaatgcagcagcgtgttcaacgtggtcaacagcagcatcggcctgattatggtgctgtccttcctgttcctgaactgatga

SEQ ID NO: 62 - RNA, 1197 nt, synthetic construct
  source 1..1197: mol_type = other RNA
atgatgagaaaactggcaattctgtctgtgtcttcttttctgtttgtggaagctctgtttcaggaataccagtgctacggatcttcttctaatacaagagtgctgaatgaactgaattacgataatgctggaacaaatctctacaatgaactggaaatgaattactacggaaaacaggaaaattggtactctctgaagaaaaattctagatctctgggagaaaatgatgatggaaataatgaagataatgaaaaactgagaaaaccaaaacacaagaaactgaagcagccagctgatggaaatccagatccaaatgcaaatccaaatgtggacccaaatgcaaatccaaatgtggacccaaatgcaaatccaaatgtggacccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgtggacccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaataaaaacaatcaaggaaatggacagggacacaatatgccaaatgatccaaatagaaatgtggatgaaaatgcaaatgcaaactctgctgtgaaaaataataataatgaagaaccatctgataagcacatcaaagaatacctgaataaaatccagaattctctgtctacagaatggtctccttgctctgtgacatgcggaaatggaatccaggtgagaatcaaaccaggatctgcaaataaaccaaaagatgaactggattatgcaaatgatattgaaaagaaaatttgcaaaatggaaaaatgcagcagcgtgttcaacgtggtcaacagcagcatcggcctgattatggtgctgtccttcctgttcctgaactgatga

SEQ ID NO: 63 - RNA, 53 nt, synthetic construct
  source 1..53: mol_type = other RNA
agaataaactagtattcttctggtccccacagactcagagagaacccgccacc

SEQ ID NO: 64 - RNA, 278 nt, synthetic construct
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 65 - RNA, 47 nt, synthetic construct
  source 1..47: mol_type = other RNA
aactagtattcttctggtccccacagactcagagagaacccgccacc

SEQ ID NO: 66 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 67 - RNA, 388 nt, synthetic construct
  source 1..388: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 68 - RNA, 10 nt, synthetic construct
  source 1..10: mol_type = other RNA
gcatatgact

SEQ ID NO: 69 - RNA, 1638 nt, synthetic construct
  source 1..1638: mol_type = other RNA
agaataaactagtattcttctggtccccacagactcagagagaacccgccaccatgatgcggaagctggccatcctgagcgtgtccagcttcctgtttgtggaagccctgttccaagagtaccagtgctacggcagcagcagcaacaccagagtgctgaacgagctgaactacgacaacgccggcaccaacctgtacaacgagctggaaatgaactactacggcaagcaagagaactggtacagcctgaagaagaacagcagaagcctgggcgagaacgacgacggcaacaacgaggacaacgagaagctgcggaagcccaagcacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatgactggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 70 - RNA, 1638 nt, synthetic construct
  source 1..1638: mol_type = other RNA
agaataaactagtattcttctggtccccacagactcagagagaacccgccaccatgatgcggaagctggccatcctgagcgtgtccagcttcctgtttgtggaagccctgttccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatccaaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatgactggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 71 - RNA, 1638 nt, synthetic construct
  source 1..1638: mol_type = other RNA
agaataaactagtattcttctggtccccacagactcagagagaacccgccaccatgatgcggaagctggccatcctgagcgtgtccagcttcctgtttgtggaagccctgtttcaggaataccagtgctacggatcttcttctaatacaagagtgctgaatgaactgaattacgataatgctggaacaaatctctacaatgaactggaaatgaattactacggaaaacaggaaaattggtactctctgaagaaaaattctagatctctgggagaaaatgatgatggaaataatgaagataatgaaaaactgagaaaaccaaaacacaagaaactgaagcagccagctgatggaaatccagatccaaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatgactggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 72 - RNA, 1638 nt, synthetic construct
  source 1..1638: mol_type = other RNA
agaataaactagtattcttctggtccccacagactcagagagaacccgccaccatgatgcggaagctggccatcctgagcgtgtccagcttcctgtttgtggaagccctgttccaggaataccagtgctatggctcaagcagcaataccagagtgctgaatgagctgaactacgacaatgccggaacaaacctgtacaacgaactcgagatgaactactatgggaaacaggagaactggtattccctgaagaagaattctcggagtttgggagaaaacgacgatggcaacaatgaggacaatgagaagcttaggaaaccgaaacacaagaagctcaaacaaccagctgatggtaatcctgatcccaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatgactggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 73 - RNA, 1638 nt, synthetic construct
  source 1..1638: mol_type = other RNA
agaataaactagtattcttctggtccccacagactcagagagaacccgccaccatgatgcggaagctggccatcctgagcgtgtccagcttcctgtttgtggaagccctgttccaggaataccagtgctatgggtcatcttccaataccagagtgctgaatgagctgaattacgacaatgccggaacaaacctgtacaacgaactggagatgaactactatggcaagcaagagaactggtatagcctcaagaagaatagcaggagtttgggtgaaaacgatgatggcaacaatgaggacaacgagaaacttcggaaacccaaacacaagaagctgaagcagcccgccgacggcaatcccgatcctaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatgactggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 74 - RNA, 1638 nt, synthetic construct
  source 1..1638: mol_type = other RNA
agaataaactagtattcttctggtccccacagactcagagagaacccgccaccatgatgcggaagctggccatcctgagcgtgtccagcttcctgtttgtggaagccctgtttcaggaataccagtgctacggatcttcttctaatacaagagtgctgaatgaactgaattacgataatgctggaacaaatctctacaatgaactggaaatgaattactacggaaaacaggaaaattggtactctctgaagaaaaattctagatctctgggagaaaatgatgatggaaataatgaagataatgaaaaactgagaaaaccaaaacacaagaaactgaagcagccagctgatggaaatccagatccaaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaataaaaacaatcaaggaaatggacagggacacaatatgccaaatgatccaaatagaaatgtggatgaaaatgcaaatgcaaactctgctgtgaaaaataataataatgaagaaccatctgataagcacatcaaagaatacctgaataaaatccagaattctctgtctacagaatggtctccttgctctgtgacatgcggaaatggaatccaggtgagaatcaaaccaggatctgcaaataaaccaaaagatgaactggattatgcaaatgatattgaaaagaaaatttgcaaaatggaaaaatgcagcagcgtgttcaacgtggtcaacagcagcatcggcctgattatggtgctgtccttcctgttcctgaactgatgactggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 75 - RNA, 1638 nt, synthetic construct
  source 1..1638: mol_type = other RNA
agaataaactagtattcttctggtccccacagactcagagagaacccgccaccatgatgaggaagcttgccatcctgtctgtcagctccttcctctttgtggaggctctgtttcaggaataccagtgctacggatcttcttctaatacaagagtgctgaatgaactgaattacgataatgctggaacaaatctctacaatgaactggaaatgaattactacggaaaacaggaaaattggtactctctgaagaaaaattctagatctctgggagaaaatgatgatggaaataatgaagataatgaaaaactgagaaaaccaaaacacaagaaactgaagcagccagctgatggaaatccagatccaaacgccaatcctaacgtggaccccaacgctaaccccaatgtggacccaaatgccaatccaaatgtcgatcccaatgcaaaccctaacgcaaacccgaatgctaacccaaacgctaatccgaacgcaaatcccaatgccaatcctaatgctaatcccaacgccaatccaaacgctaaccctaatgcaaatcctaacgctaaccccaacgcaaacccgaacgccaatcctaacgccaatccgaatgcaaaccccaatgctaaccccaatgcaaatcccaatgtggaccctaacgcaaatcctaatgcaaatccgaacgcaaatcctaacgcaaacccaaacgccaatccaaatgcaaaccctaacgctaatccaaatgctaatcccaatgcaaacccgaatgcaaatcccaacgctaaccctaacgctaaccccaatgccaatccaaacgcaaatccaaatgctaacccgaacgctaacccaaacgcaaacccaaatgctaatcctaacaagaacaatcaaggcaacggccagggccacaacatgcccaacgatcccaacagaaacgtggacgagaacgccaacgccaacagcgccgtgaagaacaacaacaatgaggaacccagcgacaagcacatcaaagagtacctgaacaagatccagaacagcctgagcaccgagtggtccccatgtagcgtgacatgcggcaacggaatccaagtgcggatcaagcctggcagcgccaacaagcctaaggatgagctggactacgccaatgacatcgagaagaaaatctgcaagatggaaaagtgcagcagcgtgttcaacgtggtcaacagcagcatcggcctgattatggtgctgtccttcctgttcctgaactgatgactggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 76 - RNA, 1638 nt, synthetic construct
  source 1..1638: mol_type = other RNA
agaataaactagtattcttctggtccccacagactcagagagaacccgccaccatgatgagaaaactggcaattctgtctgtgtcttcttttctgtttgtggaagctctgtttcaggaataccagtgctacggatcttcttctaatacaagagtgctgaatgaactgaattacgataatgctggaacaaatctctacaatgaactggaaatgaattactacggaaaacaggaaaattggtactctctgaagaaaaattctagatctctgggagaaaatgatgatggaaataatgaagataatgaaaaactgagaaaaccaaaacacaagaaactgaagcagccagctgatggaaatccagatccaaatgcaaatccaaatgtggacccaaatgcaaatccaaatgtggacccaaatgcaaatccaaatgtggacccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgtggacccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaataaaaacaatcaaggaaatggacagggacacaatatgccaaatgatccaaatagaaatgtggatgaaaatgcaaatgcaaactctgctgtgaaaaataataataatgaagaaccatctgataagcacatcaaagaatacctgaataaaatccagaattctctgtctacagaatggtctccttgctctgtgacatgcggaaatggaatccaggtgagaatcaaaccaggatctgcaaataaaccaaaagatgaactggattatgcaaatgatattgaaaagaaaatttgcaaaatggaaaaatgcagcagcgtgttcaacgtggtcaacagcagcatcggcctgattatggtgctgtccttcctgttcctgaactgatgactggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 77 - protein, 381 aa, synthetic construct
MGGAAARLGAVILFVVIVGLHGVRGQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSS

SEQ ID NO: 78 - protein, 16 aa, synthetic construct
CRKPKHKKLKQPADGN

SEQ ID NO: 79 - protein, 16 aa, synthetic construct
CHKKLKQPADGNPDPN

SEQ ID NO: 80 - protein, 20 aa, synthetic construct
CKQPADGNPDPNANPNVDPN

SEQ ID NO: 81 - protein, 16 aa, synthetic construct
CPADGNPDPNANPNVD

SEQ ID NO: 82 - protein, 16 aa, synthetic construct
CNPDPNANPNVDPNAN

SEQ ID NO: 83 - protein, 16 aa, synthetic construct
CNANPNVDPNANPNVD

SEQ ID NO: 84 - protein, 16 aa, synthetic construct
CNVDPNANPNVDPNAN

SEQ ID NO: 85 - protein, 16 aa, synthetic construct
CNVDPNANPNANPNAN

SEQ ID NO: 86 - protein, 16 aa, synthetic construct
CNANPNANPNANPNAN

SEQ ID NO: 87 - protein, 16 aa, synthetic construct
CNVDPNANPNVDPNAN

SEQ ID NO: 88 - protein, 397 aa, synthetic construct; identical to SEQ ID NO: 1

SEQ ID NO: 89 - protein, 48 aa, synthetic construct
EDNEKLRKPKHKKLKQPADGNPDPNANPNVDPNANPNVDPNANPNVDP

SEQ ID NO: 90 - protein, 24 aa, synthetic construct
NANPNANPNANPNANPNANPNANP

SEQ ID NO: 91 - protein, 397 aa, synthetic construct; identical to SEQ ID NO: 1